(19) United States
(12) Patent Application Publication
(10) Pub. No.: US 2009/0155784 A1
(43) Pub. Date: Jun. 18, 2009

(54) ASSESSMENT OF ASTHMA AND ALLERGEN-DEPENDENT GENE EXPRESSION

(75) Inventors: Margot Mary O'Toole, Newtonville, MA (US); Frederick William Immermann, Suffern, NY (US); Andrew Joseph Dorner, Lexington, MA (US); Padmalatha Sunkara Reddy, Lexington, MA (US); Holly Marie Legault, Concord, MA (US); Kerry Ann Whalen, Chelmsford, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/017,178

(22) Filed: Jan. 21, 2008

Related U.S. Application Data
(60) Provisional application No. 60/881,749, filed on Jan. 22, 2007.

(51) Int. Cl. C12Q 1/68; C40B 40/08

(52) U.S. Cl. 435/6; 506/17

(57) ABSTRACT

The present invention provides methods for the assessment, diagnosis, or prognosis of asthma including methods for providing an assessment, diagnosis, or prognosis comprising the step of exposing a sample derived from a patient to an allergen in vitro. The present invention also provides methods for selecting, as well as evaluating the effectiveness of, asthma treatments. The markers of the present invention can be used in methods to identify or evaluate agents capable of modulating marker expression levels in subjects with asthma

### CROSS-REFERENCE TO RELATED APPLICATIONS

This application claims priority from U.S. Provisional Application No. 60/881,749 filed Jan. 22, 2007. The provisional application is incorporated herein by this reference.

### TECHNICAL FIELD

The present invention relates to asthma markers and methods of using the same for the diagnosis, prognosis, and selection of treatment of asthma or other allergic or inflammatory diseases.

### BACKGROUND

Asthma is a complex, chronic inflammatory disease of the airways that is characterized by recurrent episodes of reversible airway obstruction, airway inflammation, and airway hyperresponsiveness (AHR). Typical clinical manifestations include shortness of breath, wheezing, coughing, and chest tightness that can become life threatening or fatal. While existing therapies focus on reducing the symptomatic bronchospasm and pulmonary inflammation, there is growing awareness of the role of long-term airway remodeling in accelerated lung deterioration in asthmatics. Airway remodeling refers to a number of pathological features including epithelial smooth muscle and myofibroblast hyperplasia and/or metaplasia, subepithelial fibrosis and matrix deposition. The processes collectively result in up to about 300% thickening of the airway in cases of fatal asthma. Despite the considerable progress that has been made in elucidating the pathophysiology of asthma, the prevalence, morbidity and mortality of the disease has increased during the past two decades. In 1995, in the United States alone, nearly 1.8 million emergency room visits, 466,000 hospitalizations and 5,429 deaths were directly attributed to asthma. In fact, the prevalence of asthma has almost doubled in the past 20 years, with approximately 8-10% of the U.S. population affected by the disease. (Cohn (2004) Annu. Rev. Immunol. 22:789-815) Worldwide, over four billion dollars is spent annually on treating asthma. (Weiss (2001) J. Allergy Clin. Immunol. 107:3-8)

It is generally accepted that allergic asthma is initiated by a dysregulated inflammatory reaction to airborne, environmental allergens. The lungs of asthmatics demonstrate an intense infiltration of lymphocytes, mast cells and eosinophils. This results in increased vascular permeability, smooth muscle contraction, bronchoconstriction, and inflammation. A large body of evidence has demonstrated this immune response is driven by CD4+ T-cells shifting their cytokine expression profile from T_{H}1 to a T_{H}2 cytokine profile. (Maddox (2002) Annu. Rev. Med. 53:477-98) T_{H}2 cells mediate the inflammatory response through cytokine release, including interleukins (IL) leading to IgE production and release. (Mosmann (1986) J. Immunol. 136:2348-57; Abbas (1996) Nature 383:787-93; Busse (2001) N. Engl. J. Med. 344:350-62) One murine model of asthma involves sensitization of the animal to ovalbumin (OVA) followed by intratracheal delivery of the OVA challenge. This procedure generates a T_{H}2 immune reaction in the mouse lung and mimics four major pathophysiological responses seen in human asthma, including upregulated serum IgE (atopy), eosinophilia, excessive mucus secretion, and AHR. The cytokine IL-13, expressed by basophils, mast cells, activated T cells and NK cells, plays a central role in the inflammatory response to OVA in mouse lungs. Direct lung instillation of murine IL-13 elicits all four of the asthma-related pathophysiologies and conversely, the presence of a soluble IL-13 antagonist (sIL-13Rα2-Fc) completely blocked both the OVA challenge-induced goblet cell mucus synthesis and the AHR to acetylcholine. Thus, IL-13 mediated signaling is sufficient to elicit all four asthma-related pathophysiological phenotypes and is required for the hypersecretion of mucus and induced AHR in the mouse model.

Current therapies for asthma are designed to inhibit the physiological processes associated with the dysregulated inflammatory responses associated with the diseases. Such therapies include the use of bronchodilators, corticosteroids, leukotriene inhibitors, and soluble IgE. Other treatments counter the airway remodeling occurring from bronchial airway narrowing, such as the bronchodilator salbutamol (Ventolin®), a short-acting B₂-agonist. (Barnes (2004) Nat. Rev. Drug Discov. 3:831-44; Boushey (1982) J. Allergy Clin. Immunol. 69: 335-8) The treatments share the same therapeutic goal of bronchodilation, reducing inflammation, and facilitating expectoration. Many of such treatments, however, include undesired side effects and lose effectiveness after being use for a period of time. Furthermore, current asthma treatments are not effective in all patients and relapse often occurs on these medications. (van den Toorn (2001) Am. J. Respir. Crit. Care Med. 164:2107-13) Inter-individual variability in drug response and frequent adverse drug reactions to currently marketed drugs necessitate novel treatment strategies. (Szefler (2002) J. Allergy Clin. Immunol. 109:410-8; Drazen (1996) N. Engl. J. Med. 335:841-7; Israel (2005) J. Allergy Clin. Immunol. 115:S532-8; Lipworth (1999) Arch. Intern. Med. 159:941-55; Wooltorton (2005) CMAJ 173:1030-1; Guillot (2002) Expert Opin. Drug Saf. 1:325-9) Additionally, only limited agents for therapeutic intervention are available for decreasing the airway remodeling process that occurs in asthmatics. Therefore, there remains a need for an increased molecular understanding of the pathogenesis and etiology of asthma, and a need for the identification of novel therapeutic strategies to combat these complex diseases.

Prior in vitro and in vivo studies have elucidated some critical mechanisms behind asthma pathogenesis including identifying some important mediators of allergen responsiveness. The peripheral blood mononuclear cells (PBMC) of asthmatics respond differently to stimulation with common allergens compared to healthy PBMCs in vitro. However, these studies only assessed common mediators of inflammation and immune responses such as IL-9, IL-18, IL-5, IL-4, IL-13, IL-10 and interferon (IFN)-gamma. (Devos (2006) Clin. Exp. Allergy 36:174-82; El-Mezayen (2004) Clin. Immunol. 111:61-8; Moverare (2006) Immunology 117:89-96; Moverare (1998) Allergy 53:275-81; Lagging (1998) Immunol. Lett. 60:45-9; Bottcher (2003) Pediatr. Allergy Immunol. 14(5):345-50) Although these findings are informative, they provide information for only a limited set of inflammatory targets based on known disease pathways.

### SUMMARY OF THE INVENTION

The present invention provides a new class of markers for asthma. In samples taken from patients and exposed to allergens in vitro, the expression levels of these markers respond differently in samples from patients with asthma and in samples from healthy patients. Specifically, in samples from patients with asthma, the expression levels of these markers change upon exposure to allergen, whereas comparable changes in expression are generally not observed when samples from healthy patients are similarly exposed to allergen. Accordingly, the invention provides new methods for detecting an asthma-associated biological response. The invention also provides methods for assessing an interference with an asthma-associated biological response by a treatment or potential treatment for asthma. Such a treatment can be administered to a patient, or to a sample from the patient, to assess the effectiveness of the treatment in blocking, dampening or mitigating an asthma-associated biological response by assessing the effect of the treatment on allergen-induced changes in gene expression.

The present invention provides a method for assessing an asthma-associated biological response in a sample derived from a patient. The method includes the steps of: (1) exposing the sample to an allergen in vitro; (2) detecting an expression level of at least one marker that is differentially expressed in asthma; (3) comparing the expression level to a reference expression level of the at least one marker; and (4) assessing an asthma-associated biological response based upon that comparison. In one embodiment, the at least one marker is not a cytokine gene or cytokine gene product. In another embodiment, the reference expression level of the at least one marker is the expression level of the marker in a patient sample not exposed to allergen in vitro. In one embodiment, the sample is contacted with a biological or chemical agent prior to detection of the expression level of the at least one marker to evaluate the capability of the agent to modulate the expression level of the at least one marker. In another embodiment, an asthma treatment is selected based upon the assessment made. In one embodiment, the treatment selected is one that dampens the asthma-associated biological response. In another embodiment, the at least one marker is selected from the group comprising the markers in Table 7b. In one embodiment, the at least one marker is selected from the group comprising the markers in Table 7b with an FDR for association with asthma in PBMCs prior to culture of less than 0.051.

The present invention further provides a method for diagnosis, prognosis, or assessment of asthma in a patient including the steps of: (1) exposing a sample derived from a patient to an allergen in vitro; (2) detecting an expression level of at least one marker that is differentially expressed in asthma; (3) comparing the expression level to a reference expression level of the at least one marker; (4) assessing an asthma-associated biological response based on that comparison; and (5) providing a diagnosis, prognosis, or assessment of asthma in the patient based upon the assessment of the asthma-associated biological response in the sample.

The present invention provides a method for evaluating the effectiveness of an asthma treatment in a patient including the steps of exposing the patient to the asthma treatment; exposing a sample derived from the patient to an allergen in vitro; detecting an expression level of at least one marker that is differentially expressed in asthma; comparing the expression level to a reference expression level of the at least one marker; and assessing an asthma-associated biological response based on that comparison; wherein a dampened asthma-associated biological response is indicative of the effectiveness of the asthma treatment. In one embodiment, the asthma-associated biological response is compared to an asthma-associated biological response prior to treatment. In another embodiment, the asthma-associated response is compared to a biological response in a sample derived from a healthy individual.

The present invention further provides a method for evaluating the effectiveness of an asthma treatment in a patient including the steps of: exposing a sample derived from the patient to an asthma treatment; exposing the sample to an allergen in vitro; detecting an expression level of at least one marker that is differentially expressed in asthma; comparing the expression level to a reference expression level of the at least one marker; and assessing an asthma-associated biological response based on that comparison; wherein a dampened asthma-associated biological response in a treated sample compared to an untreated sample is indicative of the effectiveness of the asthma treatment.

The present invention provides markers for asthma. Those markers can be used, for example, in the evaluation of a patient or in the identification of agents capable of modulating their expression; such agents may also be useful clinically.

Thus, in one aspect, the present invention provides a method for providing a diagnosis, prognosis, or assessment for an individual afflicted with asthma. The method includes the following steps: (1) detecting the expression levels of one or more differentially expressed genes, or markers, of asthma in a sample derived from a patient prior to the treatment; and (2) comparing each of the expression levels to a corresponding control, or reference, expression level for the marker. Diagnosis or other assessment is based, in whole or in part, on the outcome of the comparison.

In some embodiments, the reference expression level is a level indicative of the presence of asthma. In other embodiments, the reference expression level is a level indicative of the absence of asthma. In other embodiments, the reference expression level is a numerical threshold, which can be chosen, for example, to distinguish between the presence or absence of asthma. In other embodiments, the reference expression level is an expression level from a sample from the same individual but the sample is taken at a different time or is treated differently (e.g., with respect to an in vitro exposure to allergen, or allergen and an agent).

In another aspect of the present invention, what is provided is a method for diagnosing a patient as having asthma including comparing the expression level of a marker in the patient to a reference expression level of the marker and diagnosing the patient has having asthma if there is a significant difference in the expression levels observed in the comparison.

In a further aspect of the invention, what is provided is a method for evaluating the effectiveness of a treatment for asthma including the steps of (1) detecting the expression levels of one or more differentially expressed genes, or markers, of asthma in a sample derived from a patient during the course of the treatment; and (2) comparing each of the expression levels to a corresponding control, or reference, expression level for the marker, wherein the result of the comparison is indicative of the effectiveness of the treatment.

In another aspect of the present invention, what is provided is a method for selecting a treatment for asthma in a patient involving the steps of (1) detecting an expression level of a marker in a sample derived from the patient; (2) comparing the expression level of the marker to a reference expression level of the marker; (3) diagnosing the patient as having asthma; and (4) selecting a treatment for the patient.

In a further aspect of the present invention, what is provided is a method for evaluating agents capable of modulating the expression of a marker that is differentially expressed in asthma involving the steps of (1) contacting one or more cells with the agent, or optionally, administering the agent to a human or non-human mammal; (2) determining the expression level of the marker; (3) comparing the expression level of the marker to the expression level of the marker in an untreated cell or untreated human or untreated non-human mammal, the comparison being indicative of the agents ability to modulate the expression level of the marker in question.

“Diagnostic genes” or “markers” or “prognostic genes” referred to in the application include, but are not limited to, any genes or gene fragments that are differentially expressed in peripheral blood mononuclear cells (PBMCs) or other tissues of subjects having asthma as compared to the expression of said genes in an otherwise healthy individual. Exemplary markers are shown in Tables 6, 7a, 7b, 8a, and 8b. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051.

In some embodiments, each of the expression levels of the marker is compared to a corresponding control level which is a numerical threshold. Said numerical threshold can comprise a ratio, a difference, a confidence level, or another quantitative indicator.

In some embodiments, expression levels are assessed using a nucleic acid array. Typically, expression levels are assessed in the peripheral blood sample of the patient prior to, over the course of, or following a therapy for asthma.

In one embodiment, the markers include one or more genes selected from Table 6, 7a, 7b, 8a, or 8b. In another embodiment, the markers include ten or more genes selected from Table 6, 7a, 7b, 8a, or 8b. In yet another embodiment, the markers include twenty or more genes selected from Table 6, 7a, 7b, 8a, or 8b.

In another aspect, the present invention provides a method for diagnosis, or monitoring the occurrence, development, progression, or treatment of asthma. The method includes the following steps: (1) generating a gene expression profile from a peripheral blood sample of a patient having asthma; and (2) comparing the gene expression profile to one or more reference expression profiles, wherein the gene expression profile and the one or more reference expression profiles contain the expression patterns of one or more markers of asthma in PBMCs, or other tissues, and wherein the difference or similarity between the gene expression profile and the one or more reference expression profiles is indicative of the presence, absence, occurrence, development, progression, or effectiveness of treatment of the asthma in the patient. In one embodiment, the disease is asthma.

Typically, the one or more reference expression profiles include a reference expression profile representing a disease-free human. Typically, the markers include one or more genes selected from Table 6, 7a, 7b, 8a, or 8b. In some embodiments, the markers include ten or more genes selected from Table 6, 7a, 7b, 8a, or 8b.

In another aspect, the present invention provides an array for use in a method for assessing asthma in a patient. The array of the invention includes a substrate having a plurality of addresses, each of which has a distinct probe disposed thereon. In some embodiments, at least 15% of the plurality of addresses has disposed thereon probes that can specifically detect markers of asthma in PBMCs or other tissues. In some embodiments, at least 30% of the plurality of addresses has disposed thereon probes that can specifically detect markers of asthma in PBMCs or other tissues. In some embodiments, at least 50% of the plurality of addresses has disposed thereon probes that can specifically detect markers of asthma in PBMCs or other tissues. In some embodiments, the markers are selected from Tables 6, 7a, 7b, 8a, or 8b. In one embodiment, the asthma markers are selected from Table 7b. In some embodiments, the asthma markers are selected from Table 6. In one embodiment of the present invention, the asthma markers are selected from the markers indicated in Table 7b with a false discovery rate (FDR) for association with asthma in PBMCs prior to culture of less than 0.051. The probe suitable for the present invention may be a nucleic acid probe. Alternatively, the probe suitable for the present invention may be an antibody probe.

In a further aspect, the present invention provides an array for use in a method for diagnosis of asthma including a substrate having a plurality of addresses, each of which has a distinct probe disposed thereon. In some embodiments, at least 15% of the plurality of addresses has disposed thereon probes that can specifically detect markers for asthma in PBMCs or other tissues. In some embodiments, at least 30% of the plurality of addresses has disposed thereon probes that can specifically detect markers for asthma in PBMCs, or other tissues. In some embodiments, at least 50% of the plurality of addresses has disposed thereon probes that can specifically detect markers for asthma in PBMCs, or other tissues. In some embodiments, the markers are selected from Tables 6, 7a, 7b, 8a, or 8b. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051. The probe suitable for the present invention may be a nucleic acid probe. Alternatively, the probe suitable for the present invention may be an antibody probe.

In yet another aspect, the present invention provides a computer-readable medium containing a digitally-encoded expression profile having a plurality of digitally-encoded expression signals, each of which includes a value representing the expression of a marker for asthma in a PBMC, or in another tissue. In some embodiments, each of the plurality of digitally-encoded expression signals has a value representing the expression of the marker for asthma in a PBMC, or another tissue, of a patient with a known or determinable disease status. In some embodiments, the computer-readable medium of the present invention contains a digitally-encoded expression profile including at least ten digitally-encoded expression signals.

In another aspect, the present invention provides a computer-readable medium containing a digitally-encoded expression profile having a plurality of digitally-encoded expression signals, each of which has a value representing the expression of a marker for asthma in a PBMC or other tissue. In some embodiments, each of the plurality of digitally-encoded expression signals has a value representing the expression of the marker of asthma in a PBMC, or another tissue, of an asthma-free human or non-human mammal. In some embodiments, the computer-readable medium of the present invention contains a digitally-encoded expression profile including at least ten digitally-encoded expression signals.

In yet another aspect, the present invention provides a kit for prognosis of asthma. The kit includes a) one or more probes that can specifically detect markers for asthma in PBMCs, or another tissue; and b) one or more controls, each representing a reference expression level of a marker detectable by the one or more probes. In some embodiments, the kit of the present invention includes one or more probes that can specifically detect markers selected from Table 6, 7a, 7b, 8a, or 8b. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051.

In yet another aspect, the present invention provides a kit for diagnosis of asthma. The kit includes a) one or more probes that can specifically detect markers of asthma in PBMCs, or another tissue; and b) one or more controls, each representing a reference expression level of a marker detectable by the one or more probes. In some embodiments, the kit of the present invention includes one or more probes that can specifically detect markers selected from Table 6, 7a, 7b, 8a, or 8b. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051.

In one embodiment, the sample contains protein molecules from the test subject. Alternatively, the biological sample can contain mRNA molecules from the test subject or genomic DNA molecules from the test subject. An exemplary biological sample is a peripheral blood sample isolated by conventional means from a subject, e.g., blood draw. Alternatively, the sample can comprise tissue, mucus, or cells isolated by conventional means from a subject, e.g., biopsy, swab, surgery, endoscopy, bronchoscopy, and other techniques well known to the skilled artisan.

The instant invention also provides a global approach to transcriptional profiling to identify differentially responsive genes in the tissues, such as PBMCs, of asthma and healthy subjects following in vitro allergen challenge. This approach facilitates discovery of associations with asthma independent of an experimental system guided by prior knowledge of particular inflammatory mediators, and has the potential to aid in the discovery of novel markers and therapeutic candidates. Cytokine production as assessed at the protein level by different techniques, such ELISA, can be done in parallel to allow comparisons with established methods of assessing in vitro responsiveness. Global transcriptional profiling can be used to compare the effects of inhibition of asthma related targets, such cPLA2a on the in vitro response to allergen of asthma and healthy subjects.

In yet another aspect, the invention provides a method for assessing the modulating effect of an agent on an asthma-associated biological response in a sample from a patient. In one embodiment, the method comprises the steps of: (a) exposing a sample derived from a patient to an allergen in vitro; (b) detecting a level of expression of at least one marker that is differentially expressed in asthma; (c) comparing the level of expression of the at least one marker in the patient to a reference expression level of the at least one marker; and (d) assessing an asthma-associated biological response based on the comparison done in step (c), (e) exposing the sample derived from the patient to an agent; (f) detecting an expression level of the at least one marker in the sample exposed to the agent; (g) comparing the expression level of the at least one marker in the sample exposed to the agent to either (i) the expression level of the at least one marker in the sample, or (ii) the reference expression level of the at least one marker; and (h) assessing the modulation of the expression of the at least one marker by the agent. In some embodiments, the marker is not a cytokine gene or cytokine gene product. In some embodiments, a difference between the expression level of the at least one marker in the sample exposed to the agent relative to either (i) the expression level of the at least one marker in the sample, (ii) the reference expression level of the at least one marker, or both (i) and (ii), indicates that the agent modulates an asthma-associated biological response. In some embodiments, the marker is selected from the group comprising markers of Table 7b. In some embodiments, the marker is selected from a subset of the group comprising markers of Table 7b, which have a false discovery rate (FDR) for association with asthma in PBMCs prior to culture of less than 0.051.

In yet another aspect, the invention provides a method for diagnosis, prognosis or assessment of asthma in a patient. In one embodiment, the method comprises the steps of assessing an asthma-associated biological response in a sample from the patient, and providing a diagnosis, prognosis or assessment of asthma in the patient based on the assessment of the asthma-associated biological response in the sample. In some embodiments, the diagnosis, prognosis or assessment of asthma in the patient is determined by the difference between the level of expression of the at least one marker in the patient and the reference expression level of the at least one marker. In some embodiments, the reference expression level of the at least one marker is the expression level in a sample from the patient not exposed to the allergen in vitro.

In yet another aspect, the invention provides a method for evaluating the effectiveness of an asthma treatment in a patient. The method comprises the steps of: (a) exposing a first sample from the patient to the asthma treatment; (b) assessing a first asthma-associated biological response in the first sample from the patient; and (c) assessing a second asthma-associated biological response in a second sample from the patient, wherein the second sample is not exposed to the asthma treatment, and a dampened first asthma-associated biological response compared to the second asthma-associated response is indicative of the effectiveness of the asthma treatment.

In yet another aspect, the invention provides a method for asthma diagnosis, prognosis or assessment. In one embodiment, the method comprises comparing: (a) a level of expression of at least one marker in a sample from a patient, to (b) a reference level of expression of the marker, wherein the comparison is indicative of the presence, absence, or status of asthma in a patient. In some embodiments, a difference in the level of expression of the at least one marker in a sample from a patient relative to the reference level of expression of the at least one marker indicates a diagnosis, prognosis or assessment of asthma. In some embodiments, the marker is listed in Table 7b.

In yet another aspect, the invention provides a method for selecting a treatment for asthma. In one embodiment, the method comprises the steps of: (a) detecting an expression level of at least one marker in a sample derived from a patient; (b) comparing the expression level of the at least one marker in the sample derived from a patient to a reference expression level of the at least one marker; (c) determining whether the patient has asthma; and (d) selecting a treatment for the patient having asthma. In some embodiments, a difference between the expression level of the at least one marker and the reference expression level of the at least one marker determines that the patient has asthma. In some embodiments, the marker is listed in Table 7b. In some embodiments, the reference expression profile level of the at least one marker is the expression level in a sample from a healthy individual. In some embodiments the sample derived from the patient comprises peripheral blood mononuclear cells (PBMCs). In some embodiments, the treatment is any one or more of drug therapy, gene therapy, immunotherapy, radiation therapy, biological therapy, and surgery. In some embodiments, the treatment is any one or more of an anti-histamine, a steroid, an immunomodulator, an IgE downregulator, an immunosuppressant, a bronchodilator/beta-2 agonist, an adenosine A2a receptor agonist, a leukotriene antagonist, a thromboxane A2 synthesis inhibitor, a 5-lipoxygenase inhibitor, an anti-cholinergic, a K⁺ channel opener, a VLA-4 antagonist, a neurokine antagonist, theophylline, a thromboxane A2 receptor antagonist, a beta-2 adrenoceptor agonist, a soluble interleukin receptor, a 5-lipoxygenase activating protein inhibitor, an arachidonic acid antagonist, an anti-inflammatory, a membrane channel inhibitor, an anti-interleukin antibody, a PDE-4 inhibitor, and a protease inhibitor.

Other features, objects, and advantages of the present invention are apparent in the detailed description that follows. It should be understood, however, that the detailed description, while indicating embodiments of the present invention, is given by way of illustration only and not by way of limitation. Various changes and modifications within the scope of the invention will become apparent to those skilled in the art from the detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings are provided for illustration, and do not constitute a limitation.

FIG. 1 is an illustration of gene expression profiling. FIG. 1 provides a visualization of the allergen-dependent expression pattern of 167 probesets that differ significantly between asthma and healthy subjects: Subjects are shown in columns, and genes in rows. Red indicates an allergen-dependent change higher than the mean. Green indicates an allergen-dependent change lower than the mean. An unsupervised clustering algorithm, which determines similarities between subjects independent of group membership, was used to generate this visualization. Subjects are grouped according to the degree of similarity in expression pattern. Note that, with one exception, the 11 healthy volunteers are grouped together, and that, with 4 exceptions, the 26 asthma subjects group together.

FIG. 2 is an illustration of gene expression profiling. Gene expression profiling demonstrates differential modulation of 167 probes in the asthma subjects in response to allergen in the presence of the cPLA2a inhibitor 4-{3-[1-benzhydryl-5-chloro-2-(2-{[(2,6-dimethylbenzyl) sulfonyl]amino}ethyl)-1H-indol-3-yl]propyl}benzoic acid. An unsupervised clustering algorithm, which determines similarities between subjects independent of group membership, was used to generate this visualization. Subjects are shown in columns, and genes in rows. Red indicates an allergen-dependent change higher than the mean. Green indicates an allergen-dependent change lower than the mean. Subjects are grouped according to the degree of similarity in expression pattern: H—healthy volunteer allergen dependent fold change, A—asthmatic allergen dependent fold change. A+—Effect of the cPLA2a inhibitor on allergen dependent fold change.

FIG. 3 is an illustration of network profiles. Network profiles were generated by Ingenuity pathways analysis (Ingenuity Systems, Mountain View, Calif.). The top scoring Network, Network 1, consisted of 34 nodes, representing genes. Nodes are color coded according to whether they were upregulated (red) or downregulated (green). (A) Functional analysis of Network 1, colored in relation to the asthma specific-allergen response; (B) Network 1, colored in relation to the healthy volunteer response to allergen; (C) Functional analysis, Network 1, colored in relation to asthma specific cPLA2 inhibitor 4-{3-[1-benzhydryl-5-chloro-2-(2-{[(2,6-dimethyl benzyl)sulfonyl]amino}ethyl)-1H-indol-3-yl]propyl}benzoic acid response in the presence of allergen.

### DETAILED DESCRIPTION

The present invention provides a new class of markers that are differentially expressed in asthma, particularly in peripheral blood mononuclear cells. In particular, the markers of the present invention, when exposed to allergens in vitro, are differentially expressed in samples derived from asthmatics as compared to samples derived from healthy volunteers. Specifically, the markers of the present invention upregulate or downregulate their expression in asthmatics to a greater extent when exposed to allergens in vitro than they do in healthy individuals. The present invention provides methods for assessing an asthma-associated biological response in a sample derived from a patient by exposing the sample to allergen in vitro and comparing the expression level of one or more markers with a reference expression level of the one or more markers. The invention also provides methods for selecting an asthma treatment based upon an assessment of an asthma-associated biological response in a sample derived from a patient after exposing the sample to allergen in vitro and comparing the expression level of one or more markers with a reference expression level of the one or more markers.

Also provided by the present invention are methods for evaluating the capability of a biological or chemical agent to modulate the expression levels of one or more markers based upon an assessment of an asthma-associated biological response which is assessed after exposing a patient-derived sample to an allergen in vitro and comparing the expression level of one or more markers with a reference expression level of the one or more markers. The present invention provides methods for diagnosis, prognosis, or assessment of asthma in a patient in which an asthma-associated biological response is assessed by exposing a patient-derived sample to allergen in vitro and comparing the expression levels of one or more markers to a reference expression level of the one or more markers, with subsequent use of this assessment to provide a diagnosis, prognosis, or assessment of asthma in the patient. Also provided by the present invention are methods for evaluating the effectiveness of an asthma treatment in a patient in which a patient is exposed to an asthma treatment and an asthma-associated biological response is assessed as previously described, with a dampened asthma-associated biological response indicating the effectiveness of the asthma treatment.

The present invention also provides methods for asthma diagnosis, prognosis, or assessment in which the expression level of one or more markers of the present invention is compared to a reference level of the one or more markers. Further provided by the present invention are methods for evaluating the effectiveness of an asthma treatment in a patient in which the expression level of one or more markers of the present invention is detected and compared to a reference expression of the one or more markers. The present invention provides a method for selecting a treatment for asthma in which the expression level of one or more markers of the present invention is detected, compared to a reference expression level of the one or more markers, a diagnosis of the patient as having asthma is made, and a treatment for the patient is selected. Also provided by the present invention are methods for identifying or evaluating agents capable of modulating the expression levels of at least one marker of the present invention in which cells derived from subjects, or subjects themselves, are exposed to an agent and the expression levels of one or more markers are determined and compared to reference expression levels for the one or more markers, the comparison being indicative of the capability of the agent to modulate the expression levels of the one or more markers. The present invention represents a significant advance in clinical asthma pharmacogenomics and asthma treatment.

Various aspects of the invention are described in further detail in the following subsections. The use of subsections is not meant to limit the invention. Each subsection may apply to any aspect of the invention. In this application, the use of “or” means “and/or” unless stated otherwise.

### In Vitro Allergen Challenge

The present invention provides methods for diagnosis, prognosis, or assessment of a patient's asthma comprising the steps of (1) exposing a sample derived from a patient to an allergen in vitro; (2) detecting the expression level of at least one marker that is differentially expressed in asthma; (3) comparing the expression level of the at least one marker in the patient with a reference expression level of the at least one marker; and (4) providing a diagnosis, prognosis, or assessment of the patient's asthma condition or state using the comparison performed in step (3). In particular, the method also provides for the use of the provided diagnosis, prognosis, or assessment in conjunction with selecting a treatment for a subject's asthma, or evaluating the effectiveness of an agent in modulating the expression of one or more markers differentially expressed in asthma. In one embodiment of the present invention, the agent modulates the expression of level of the one or more markers to the expression level of the marker or markers in a healthy subject. In another embodiment of the present invention, the agent modulates the asthma phenotype to a healthy phenotype. Samples may be exposed to an allergen singly or multiply, as in a cocktail, in any and all forms and manners known to the skilled artisan including, but not limited to, in solution, lyophilized, in an aerosol, in an emulsion, in a micelle, in a microsphere, in a colloidal suspension, etc. Allergens may be, but are not limited to being, recombinant, purified, solid-state synthesized, or derived from any other commonly known and used method within the art for procuring, generating, or deriving allergens. Allergens can be organic or inorganic molecules, and can be, but are not limited to being, from food, from fibers, from insects, from animals, from plants, and, in particular, can be, but are not limited to being, from house dust mite, from ragweed, from cat, or may be generated in recombinant form or procured in recombinant form commercially. The allergen may be provided to a sample and in any and all quantities and concentrations the skilled artisan would understand to be effective to elicit a response by a sample in vitro. The practice of the use of allergens in the use of this method is well within the skill in the art and the skilled artisan would understand what variations and modifications are possible within the scope of this method.

### Identification of Asthma Markers Using HG-U133A Microarrays

A study was conducted to investigate (a) how effects of in vitro exposure to allergen differ between asthma and healthy subjects, and (b) the involvement of the cPLA2a pathway in the process identified as different between the two groups. In addition, the study was intended to identify potential new targets and/or markers for asthma. The approach to the answers to these questions involved seeking to identify differences between the healthy and asthmatic phenotypes at the molecular level. Transcriptional profiling methods have been employed as an exploratory screen independent of pre-existing disease paradigms (Bennett (2003) Exp. Med. 197:711-23; Bovin (2004) Immunol. Lett. 93:217-26; Burczynski (2006) J. Mol. Diagn. 8:51-61). Our investigations have revealed heretofore unrecognized associations between a number of genes and asthma in circulating PBMCs in vivo in the absence of allergen stimulation. Our results also provide an indication of qualitative differences in response to allergen between healthy and asthmatic phenotypes. We have identified many significant allergen-dependent gene expression differences between the asthma and healthy groups, and those differences are the focus of this study. We have extended this analysis further to include the effects of inhibition of the cPLA2a pathway on gene expression patterns significantly associated with the asthma group.

The cytosolic form of phospholipase 2 (cPLA2) catalyzes the first step in the biosynthesis of inflammatory lipid mediators, the eiconasoids (Leslie (1997) J. Biol. Chem. 272:16709-12) and is theoretically an attractive target for inhibition in the treatment of inflammatory diseases. The in vitro allergen challenge is a model system to evaluate the effects of cPLA2 inhibition in blood cells, including PBMCs.

Transcriptional profiling was done on RNA collected from allergen treated PBMCs from the asthmatic and healthy volunteers and gene expression levels were measured as described above. There were 10280 probesets that were called present in at least 5 samples and a frequency greater than 10 ppm and these were selected for further analysis. From these we identified the genes that showed a similar response to allergen in both the asthmatic and healthy groups. Genes in this category had an allergen dependent fold change ≧1.5, and had no significant difference (FDR≧0.051) between the two groups with respect to allergen-dependent changes. There were 133 probesets (representing 123 unique genes) that met these criteria. The complete list of probes and their descriptions are included in Table 7a. The fourth column of Table 7a indicates the FDR for the significance of the association of genes with asthma in PBMCs prior to culture (that is, untreated PBMCs) when profiles were compared between asthmatics and healthy volunteers. Genes that were up regulated in both populations included those involved in the immune response and cell growth. For example, interleukin-9 (IL9) (Godfraind (1998) J. Immunol. 160:3989-96; Louahed (2001) Blood 97:1035-42; Temann (1998) J. Exp. Med. 188:1307-20; Vink (1999) J. Exp. Med. 189:1413-23) and chemokine (C-X-C motif) ligand 3 (CXCL3) (Geiser (1993) J. Biol. Chem. 268:15419-24; Inngjerdingen (2001) Blood 97:367-75) are immune system genes that are involved in chemotaxis and activation of lymphoid cells that are up-regulated in both populations but were up-regulated to a greater extent in the asthma subjects. Genes down-regulated in response to allergen included those implicated in degradation of the extracellular matrix, matrix metalloproteases-2 and 12 (MMP2, MMP12) (Sternlicht (2001)Annu. Rev. Cell Dev. Biol. 17:463-516).

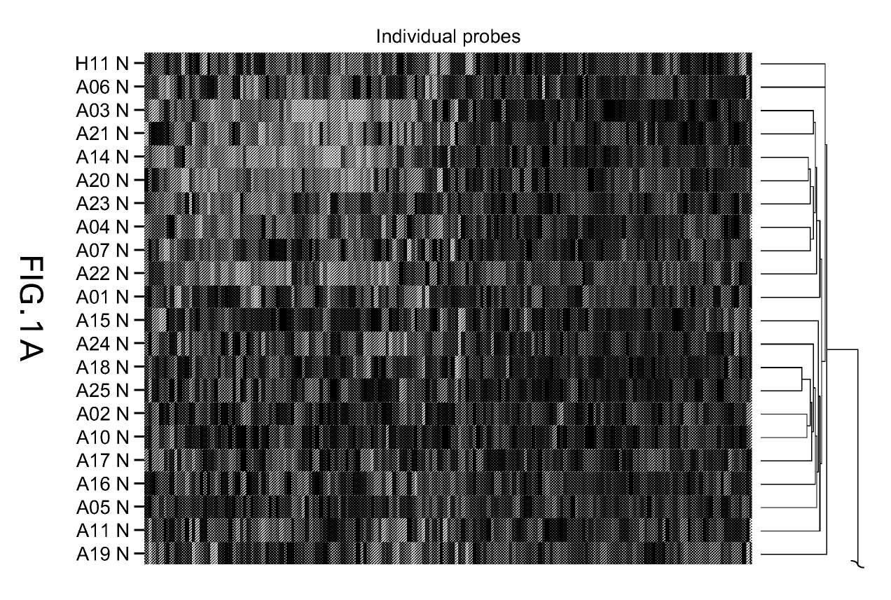

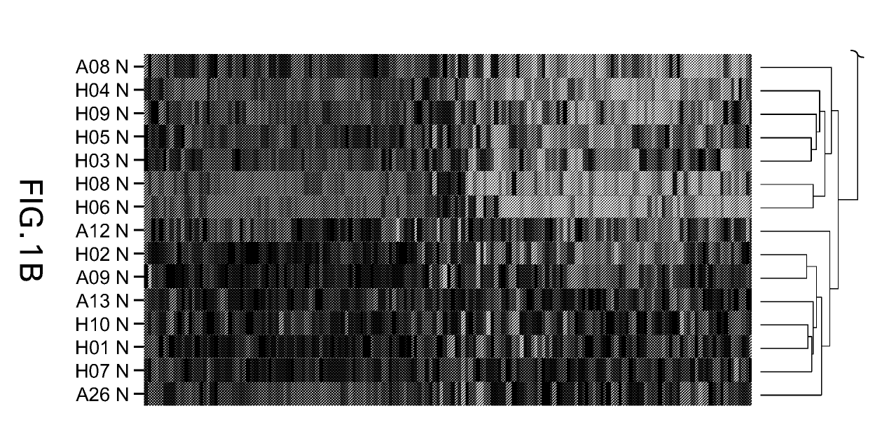

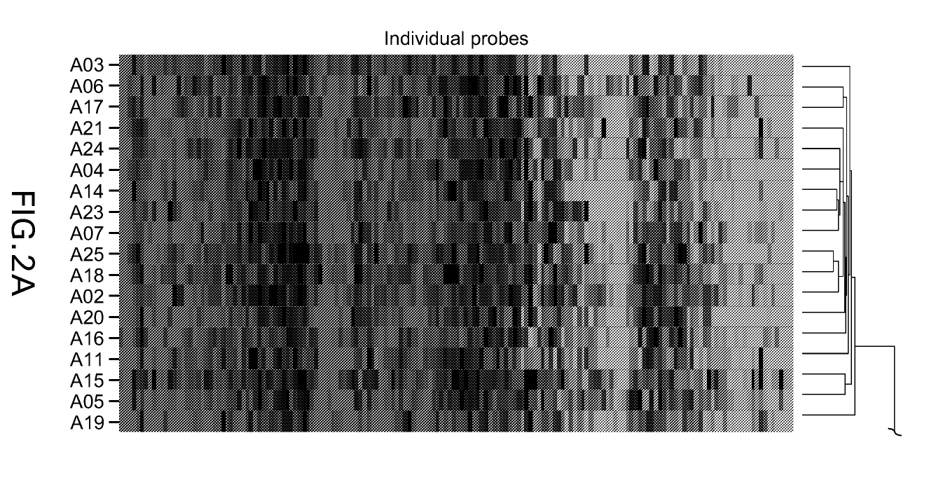

Comparison of the expression levels of the 10280 probesets in the asthma and healthy subjects identified 167 probesets (representing 153 unique genes) whose allergen-dependent changes differed significantly (FDR<0.051) between asthma and healthy subjects. These genes also showed an allergen-dependent fold change >1.5 in at least one group. The complete list of the 167 probe sets and, for each, the significance of the difference between the groups is shown in Table 7b. The fourth column of Table 7b indicates the FDR for the significance of the association of genes with asthma in PBMCs prior to culture (that is, untreated PBMCs) when profiles were compared between asthmatics and healthy volunteers. A visualization of the differences between asthma and healthy subjects with respect to allergen-dependent changes in expression level of all 167 probesets is shown in FIG. 1. The visualization was generated using an algorithm that groups subjects based on the similarities with respect to allergen dependent gene expression changes. With one exception, all the healthy subjects were grouped together, and 22 of the 26 asthma subjects were grouped together. Table 6 shows 50 genes—a subset of genes that showed a significant difference between asthma and healthy subjects with respect to the response to allergen. The genes shown in Table 6 were associated with an allergen response of 1.5 fold or more in the asthma group (asthma subjects (AOS)), while having a less than 1.1 fold response to allergen in the healthy volunteer population (WHV), having an FDR cutoff of <0.051. According to Table 6, panel (A) depicts genes up regulated in asthma subjects 1.5 fold or higher compared to healthy volunteers; panel (B) depicts genes down regulated by 1.5 fold or more in asthma subjects compared to healthy volunteers.

In this list of Table 6 are genes previously associated with the asthmatic phenotype including the Zap70 and LCK tyrosine kinases (Wong (2005) Curr. Opin. Pharmacol. 5:264-71), the toll like receptor 4 (TLR4) (Hollingsworth (2004) Am. J. Respir. Crit. Care Med. 170:126-32; Rodriguez (2003) J. Immunol. 171:1001-8), and complement component 3a receptor 1 (C3AR1). (Bautsch (2000) J. Immunol. 165:5401-5; Drouin (2002) J. Immunol. 169:5926-33; Hasegawa (2004) Hum. Genet. 115:295-301; Humbles (2000) Nature 406:998-1001; Zimmermann (2003) J. Clin. Invest. 111:1863-74) Accordingly, in some embodiments of the invention, at least one marker is detected other than one of the genes previously associated with asthma. Allergen-responsive genes not previously shown to be involved in the asthma phenotype included sialoadhesin (SN1-CD163) (Fabriek (2005) Immunobiology 210:153-60), interleukin-21 receptor (IL21R) (Mehta (2004) Immunol. Rev. 202:84-95), and a disintegrin/metalloprotease, ADAM19 (Fritsche (2000) Blood 96:732-9).

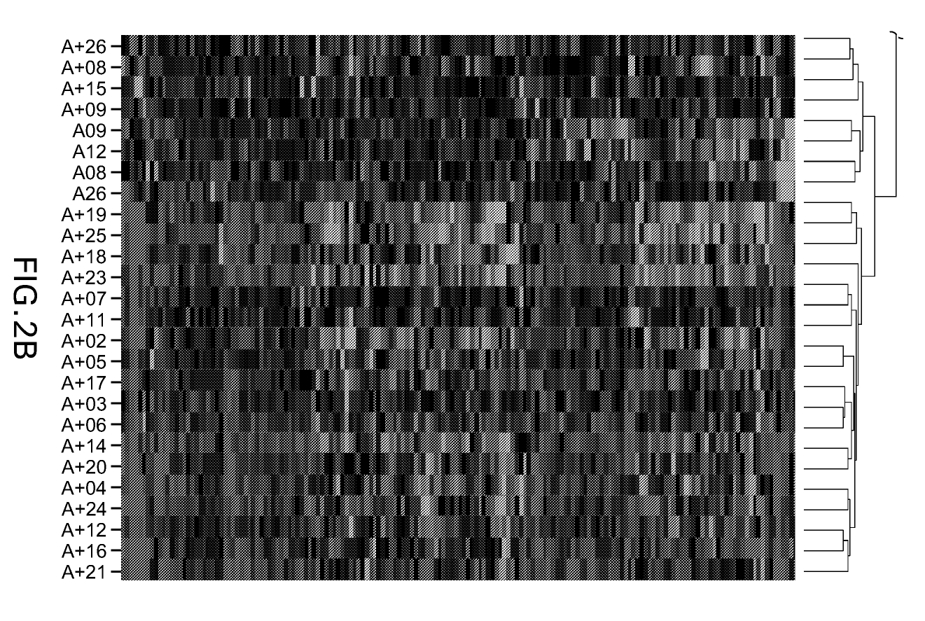

The transcriptional effect of cPLA2 inhibition on expression of the 167 allergen-asthma specific probesets was determined. The asthma specific gene expression was altered in the presence of the inhibitor (4-{3-[1-benzhydryl-5-chloro-2-(2-{[(2,6-dimethylbenzyl) sulfonyl]amino}ethyl)-1H-indol-3-yl]propyl}benzoic acid) (hereinafter “the cPLA2 inhibitor”) when compared to the allergen treatment alone. The complete analysis results, including fold changes, with and without cPLA2 inhibition are provided in Tables 7a and 7b. With the exception of a few probes, the probe set falls into two distinct categories. In the first category, probes that correspond to genes that were up-regulated in asthma samples in response to allergen, such as ZAP70, LCK, and MCM2, are reduced to the levels seen in the allergen treated healthy controls. In the second category, genes that were initially down regulated in the asthma samples in the presence of allergen, such as sialoadhesin (SN), CD84, and tissue inhibitor of metalloproteinase 3 (TIMP3) are up-regulated in the presence of inhibition. A hierarchical cluster analysis was performed to visualize the differences associated with cPLA2a inhibition for the 167 asthma-associated probe sets (see FIG. 2). The analysis identified three separate groups based on similarities in gene expression pattern: 1) asthma samples treated with allergen, 2) asthma samples treated with allergen and the cPLA2 inhibitor and 3) a small population of samples allergen treated and allergen+the cPLA2 inhibitor treated. Interestingly, group 3 contains the same subjects who originally clustered with the healthy samples in response to allergen (see FIG. 1).

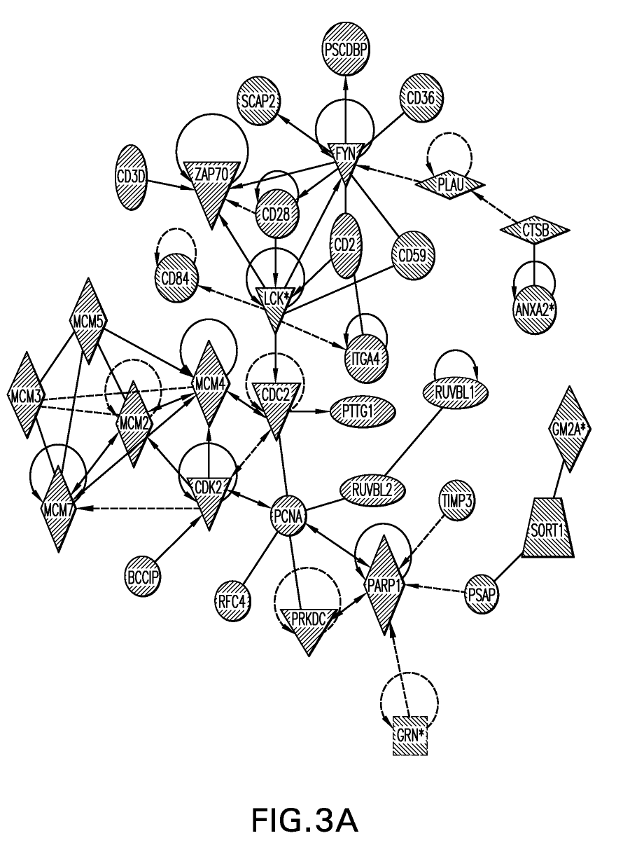

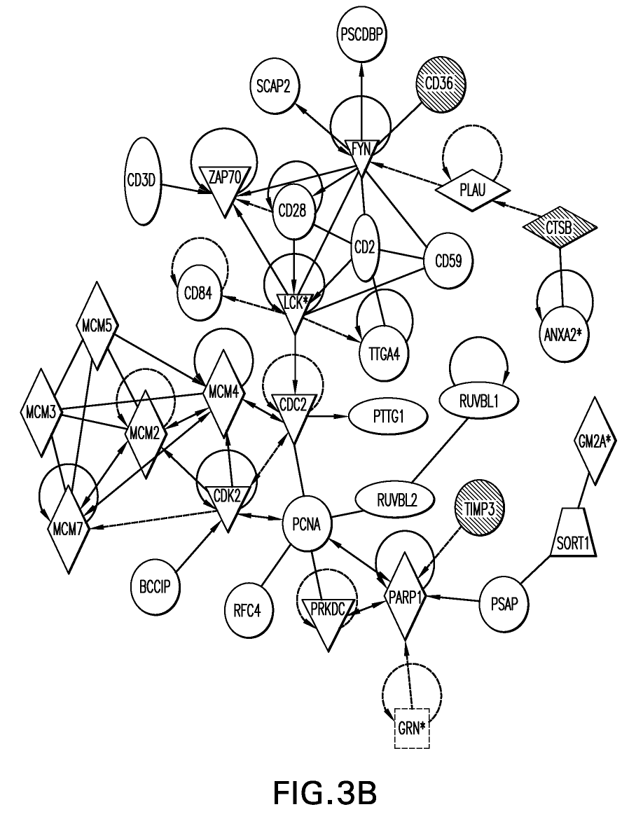

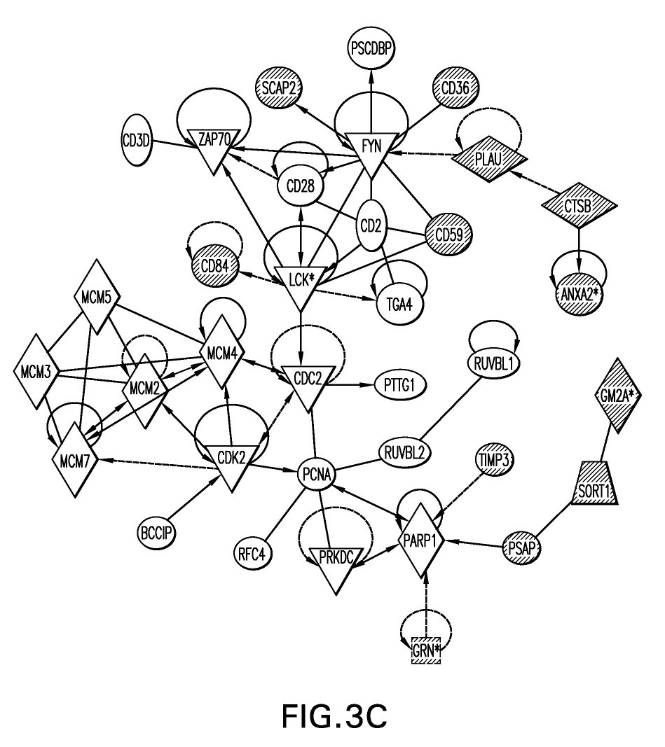

To explore the functional relatedness of the allergen-responsive genes and identify associated pathways, the asthma-specific allergen gene list, (167 probeset) was functionally annotated by Ingenuity Pathways Analysis (IPA). In this analysis, the expression values obtained in the presence of the inhibitor were overlaid into the gene set created based on asthma specific allergen gene changes. Of the 167 probes initially entered into the analysis, 127 met the criteria for pathway analysis. The criteria are based on the Ingenuity knowledge base and on our previous statistical analysis. Seven well-populated functional networks were created based on this information. The top functions for the networks created using IPA include immune and lymphatic system development and function, immune response, DNA replication, recombination and repair. The top-scoring network (Network 1) consisted of 35 nodes that represent genes involved in immune response and cell cycle (FIG. 3A). Genes in this network involved in the immune response were upregulated in the asthmatics compared to the healthy subjects including the T cell receptor signaling genes CD3D, CD28, and ZAP70 (Kuhns (2006) Immunity 24:133-9; Wang (2004) Cell Mol. Immunol. 1:37-42; Zamoyska (2003) Immunol. Rev. 191:107-18). As expected, the expression levels (node color intensities) in Network 1 for the healthy volunteer population looked very different from the asthma subjects. Every single probe in Network 1 in the asthmatic population has an altered level of expression in the presence of the inhibitor (FIG. 3C). However, in the healthy subjects, a few of the genes were downregulated similarly to the asthma subjects, but to a significantly lesser extent. This set of genes includes cathepsin B (CTSB), tissue inhibitor of metalloproteinase 3 (TIMP3) and CD36 antigen (collagen type I receptor, thrombospondin receptor) (CD36) (FIG. 3B). In the healthy population, the few genes that were down regulated in response to allergen in Network 1 are brought up to non-allergen-stimulated background levels in the presence of the inhibitor (data not shown).

As shown in FIG. 3C, all T cell responsive and cell cycle genes in the pathway depicted in FIG. 3A were significantly changed towards the levels in the healthy subject group by cPLA2a inhibition. Allergen challenge increased expression of the T cell genes ZAP70, CD28 and CD3D (FIG. 3B), and this increase was abolished with cPLA2a inhibition (FIG. 3C). This result is noteworthy given that CD4+ T cells are believed critical for the development and maintenance of the disease. Other immune related genes were also downregulated by cPLA2a inhibition including, the CD antigens CD28 and CD3D, IL-21R and the transcription factor, high-mobility group box 1 protein, HMGB1. The HMGB1 result is of particular interest as this protein has been shown to be a distal mediator of acute inflammation of the lung linked to an increased production of pro-inflammatory cytokines (Abraham (2000) J. Immunol. 165:2950-4). The effects of cPLA2 inhibition on allergen-related, asthma-associated expression levels are further illustrated in Tables 7a and 7b.

Inhibition of cPLA2 does not affect gene expression in the absence of allergen stimulation in the asthmatic population. Only three genes met the filtering cut off of an FDR less than equal to 0.051 and 1.5 or greater fold change (Table 8a), representing an unknown gene, a pituitary specific gene, PACAP, and a hormone, PMCH. In the healthy population, 36 probes were significantly upregulated in the presence of cPLA2 inhibition and 43 probes were significantly upregulated in the presence of cPLA2 and 43 probes were significantly downregulated in the presence of cPLA2 inhibition (Table 8b).

The specific allergens used in this study are common environmental antigens and there were many similarities in the in vitro responses to allergen among asthma and healthy subjects. The in vitro cytokine response as measured by ELISA was comparable, and many allergen-dependent gene expression changes were not significantly different between the two groups. Given the robust allergen responses that did not differ significantly between asthma and healthy subjects, the standard of care treatment that the asthma subjects were receiving did not prevent robust responses in this 6-day culture experimental system. Among genes with comparable responses to allergen in asthma and healthy subjects are chemokines and interleukins, some of which have previously been associated with the asthma phenotype including those involved in the T cell response such as interleukin-17 (Molet (2001) J. Allergy Clin. Immunol. 108:430-8; Sergejeva (2005) Am. J. Respir. Cell Mol. Biol. 33:248-53) and IL-9 (Erpenbeck (2003) J. Allergy Clin. Immunol. 111:1319-27; Temann (1998) J. Exp. Med. 188:1307-20). In general, genes that have previously been shown to be involved in the asthma subject response were modified to a greater extent in the asthma as compared to the healthy group in response to allergen. For example, the chemokine ligand 1 (CCL1) (Montes-Vizuet (2006) Eur. Respir. J. 28(1):59-67) and the chemokine ligand 18 (CCL18) (de Nadai (2006) J. Immunol. 176:6286-93) have recently been shown to be involved in the asthmatic phenotype and are upregulated to a greater extent in the asthmatic population. Also contained within this gene set were genes not involved in the immune response, including those involved in protective stress responses such as methallothionein (MT) gene family, MT2A and MT1X (Thornalley (1985) Biochim. Biophys. Acta 827:36-44; Andrews (2000) Biochem. Pharmacol. 59:95-104) as well as those involved in glucose transport, GLUT-3 and GLUT-5 (Olson (1996) Annu. Rev. Nutr. 16:235-56; Seatter (1999) Pharm. Biotechnol. 12:201-28).

The identification of a relatively large subset of genes that distinguish between asthma and healthy subjects underscores the power of the global profiling approach in elucidating differences between groups that had not been previously observed. In fact, despite the standard of care therapy that the asthma subjects were receiving, several genes were identified that were previously shown to be involved in the asthma phenotype. These include complement component 3a receptor 1 (C3AR1) (Drouin (2002) J. Immunol. 169:5926-33; Humbles (2000) Nature 406:998-1001; Zimmermann (2003)J. Clin. Invest. 111:1863-74; Bautsch (2000) J Immunol. 165:5401-5; Hasegawa (2004) Hum. Genet. 115:295-301) and the toll like receptor (TLR4) (Rodriguez (2003) J. Immunol. 171:1001-8; Hollingsworth (2004) Am. J. Respir. Crit. Care Med. 170:126-32). C3AR1 is the receptor for the complement component 3a (C3a) and is involved in T_{H}2 inflammatory responses (Ames (1996) J. Biol. Chem. 271:20231-4; Crass (1996) Eur. J. Immunol. 26:1944-50; Drouin (2002) J. Immunol. 169:5926-33). C3AR knockout mice challenged with allergens have a decrease in airway hyperresponsiveness, airway eosinophils, and IL-4 producing cells relative to wild type mice (Drouin (2002) J. Immunol. 169:5926-33). The data demonstrate that, under these in vitro conditions (6 days in culture), the toll like receptor 4 (TLR4) was differentially modulated in asthma subjects in the presence of allergen. The toll-like receptors are a family of proteins that enhance certain cytokine gene transcription in response to pathogenic ligands (Medzhitov (2001) Nat. Rev. Immunol. 1:135-45; Akira (2001) Nat. Immunol. 2:675-80). TLR4 responds to LPS (Perera (2001) J. Immunol. 166:574-81; Takeda (2003) Annu. Rev. Immunol. 21:335-76) and recent evidence suggests that TLR4 is important in the asthma phenotype, although the data are conflicting (Rodriguez (2003) J. Immunol. 171:1001-8; Savov (2005) Am. J. Physiol. Lung Cell Mol. Physiol. 289(2):L329-37). The discrepancies may be attributable to differences in experimental systems (Eisenbarth (2002) J. Exp. Med. 196:1645-51). Despite discrepancies in the literature, the results implicate TLR4 as associated with the asthma subject in vitro response to allergen.

The majority of the 167 differentially regulated probes, approximately 80%, have not been previously shown to be involved in the asthma phenotype. Among these are the ATPase transporters, ATP6V0D1, ATP6V1A, and ATP6AP1 and the CD antigens, CD163, CD169, CD84, CD59 and PRNP, which is expressed in a variety of immune cell types. Macrophages obtained from mice that do not express PRNP have higher rates of phagocytosis than the wild-type cells in vitro (de Almeida (2005) J. Leukoc. Biol. 77:238-46). Therefore, regulation of PRNP could be important for the activation of macrophages in the asthma group. Available data on the importance of macrophages in the asthmatic phenotype does not indicate the significance of macrophage PRNP in the asthma phenotype (Peters-Golden (2004) Am. J. Respir. Cell Mol. Biol. 31:3-7). However, alveolar macrophages play a role in innate immune responses and these responses have been shown to affect the severity of asthma and bronchoconstriction in asthma (Broug-Holub (1997) Infect. Immun. 65:1139-46; Michel (1989) J. Appl. Physiol. 66:1059-64; Michel (1996) Am. J. Respir. Crit. Care Med. 154:1641-6).

Genes modulated in the allergen-treated PBMCs of asthma subjects that have not previously been associated with asthma also include the mini-chromosome maintenance proteins (MCM) MCM2, MCM5, and MCM7 along with polycomb group ring finger 4 protein, BMI1. BMI1 is involved in lymphoproliferation and is implicated in T cell differentiation, and, therefore the lymphoproliferative effect of BMI1 could be important for the asthmatic phenotype, perhaps by playing a role in increasing the amount of CD4+ T cells in the lungs of asthmatics (Alkema (1997) Oncogene 15:899-910; Raaphorst (2001) J. Immunol. 166:59 25-34; Robinson (1992) N. Engl. J. Med. 326:298-304)

Our investigations also indicated that many of the probesets identified in Tables 7a and 7b are surprisingly and significantly associated with asthma in circulating PBMCs in vivo even in the absence of allergen stimulation. The fourth column of Tables 7a and 7b provides the FDR for the significance of the association of genes with asthma in PBMCs prior to culture (that is, untreated PBMCs). Genes not having a significant association with asthma in circulating PBMCs did not pass this PBMC analysis filter and are identified accordingly.

Using the methods of the present invention, it was also possible to determine the effectiveness of treating asthmatics with a specific enzyme inhibitor, or any other agent.

Use of the methods and precepts of the present invention allows the skilled artisan to conduct a comprehensive molecular analysis of human tissue for asthma associated genes/markers for responses to drugs used to treat such disease. Such analysis can lead to insights into treatment targets and better diagnoses. Global transcriptional profiling can be used as a sensitive exploratory tool to study the molecular mechanisms of asthma and responses to drugs used to treat them without relying on pre-existing paradigms. Thus, the methods of the present invention have the potential to lead to the discovery of novel targets and biomarkers. In the clinical setting, target disease tissue is often difficult to obtain from patients and thus surrogates to the most proximal disease must be examined. Peripheral blood is an easily accessible tissue and the transcriptome of peripheral blood mononuclear cells (PBMCs) can be studied both directly upon collection and following in vitro stimulation. What has been described herein, and in the examples, is an in vitro model system using fresh whole blood to study the response of PBMCs from asthma subjects and healthy subjects to identify disease-related transcriptional profiles and to model the response of PBMCs in the clinical setting to drug exposure using an experimental inhibitor of cPLA2. The results of this global profiling study have uncovered differences and similarities between asthma and healthy subjects as revealed by in vitro allergen responsiveness. In part because of its scope and size, the study has confirmed some previously reported asthma associations, has shown that other previously reported associations are not as significant as was thought from smaller studies, and has discovered novel associations that were not predictable based on the pre-existing information. These results clearly demonstrate that global transcriptional profiling has utility as a sensitive exploratory tool to study molecular mechanisms of disease and pathways affected by candidate therapeutics. The preceding description provides guidance by way of illustration, and not limitation, as to the methods of the present invention.

As discussed earlier, expression level of markers of the present invention can be used as an indicator of asthma. Detection and measurement of the relative amount of an asthma-associated marker or marker gene product (polynucleotide or polypeptide) of the invention can be by any method known in the art.

Methodologies for detection of a transcribed polynucleotide can include RNA extraction from a cell or tissue sample, followed by hybridization of a labeled probe (i.e., a complementary polynucleotide molecule) specific for the target RNA to the extracted RNA and detection of the probe (i.e., Northern blotting).

Methodologies for peptide detection include protein extraction from a cell or tissue sample, followed by binding of an antibody specific for the target protein to the protein sample, and detection of the antibody. Antibodies are generally detected by the use of a labeled secondary antibody. The label can be a radioisotope, a fluorescent compound, an enzyme, an enzyme co-factor, or ligand. Such methods are well understood in the art.

Detection of specific polynucleotide molecules may also be assessed by gel electrophoresis, column chromatography, or direct sequencing, quantitative PCR, RT-PCR, or nested PCR among many other techniques well known to those skilled in the art.

Detection of the presence or number of copies of all or part of a marker as defined by the invention may be performed using any method known in the art. It is convenient to assess the presence and/or quantity of a DNA or cDNA by Southern analysis, in which total DNA from a cell or tissue sample is extracted, is hybridized with a labeled probe (i.e., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as would be understood by one skilled in the art.

### Diagnosis, Prognosis, and Assessment of Asthma

The asthma markers disclosed in the present invention can be employed in diagnostic methods comprising the steps of (a) detecting an expression level of an asthma marker in a patient; (b) comparing that expression level to a reference expression level of the same asthma marker; (c) and diagnosing a patient has having, nor having asthma, based upon the comparison made. The methods described herein below, including preparation of blood and other tissue samples, assembly of class predictors, and construction and comparison of expression profiles, can be readily adapted for the diagnosis of, assessment of, and selection of a treatment for asthma. This can be achieved by comparing the expression profile of one or more asthma markers in a subject of interest to at least one reference expression profile of the asthma markers. The reference expression profile(s) can include an average expression profile or a set of individual expression profiles each of which represents the gene expression of the asthma markers in a particular asthma patient or disease-free human. Similarity between the expression profile of the subject of interest and the reference expression profile(s) is indicative of the presence or absence of the disease state of asthma. In many embodiments, the disease genes employed for the diagnosis or monitoring of asthma are selected from the markers described in Tables 6, 7a, 7b, 8a, and/or 8b. One or more asthma markers selected from Tables 6, 7a, 7b, 8a, and/or 8b can be used for asthma diagnosis or disease monitoring. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051. In one embodiment, each asthma marker has a p-value of less than 0.01, 0.005, 0.001, 0.0005, 0.0001, or less. In another embodiment, the asthma genes/markers comprise at least one gene having an “Asthma/Disease-Free” ratio of no less than 2 and at least one gene having an “Asthma/Disease-Free” ratio of no more than 0.5. A diagnosis of a patient as having asthma can be established under a range of ratios, wherein a significant difference can be ratio of the asthma marker expression level to healthy expression level of the marker of >|1| (absolute value of 1). Such significantly different ratios can include, but are not limited to, the absolute values of 1.001, 1.01, 1.05, 1.1, 1.2, 1.3, 1.5, 1.7, 2, 3, 4, 5, 6, 7, 10, or any and all ratios commonly understood to be significant by the skilled practitioner.

The asthma markers of the present invention can be used alone, or in combination with other clinical tests, for asthma diagnosis or disease monitoring. Conventional methods for detecting or diagnosing asthma include, but are not limited to, blood tests, chest X-ray, biopsies, skin tests, mucus tests, urine/excreta sample testing, physical exam, or any and all related clinical examinations known to the skilled artisan. Any of these methods, as well as any other conventional or non-conventional method, can be used, in addition to the methods of the present invention, to improve the accuracy of asthma diagnosis or monitoring.

The markers of the present invention can also be used for the prediction of the diagnosis, assessment, or prognosis of an asthma patient of interest. The prediction typically involves comparison of the peripheral blood expression profile, or expression profile from another tissue, of one or more markers in the asthma patient of interest to at least one reference expression profile. Each marker employed in the present invention is differentially expressed in peripheral blood samples, or other tissue samples, of asthma patients who have different assessments.

In one embodiment, the markers employed for providing a diagnosis are selected such that the peripheral blood expression profile of each marker is correlated with a class distinction under a class-based correlation analysis (such as the nearest-neighbor analysis), where the class distinction represents an idealized expression pattern of the selected genes in tissue samples, such as peripheral blood samples, of asthma patients and healthy volunteers. In many cases, the selected markers are correlated with the class distinction at above the 50%, 25%, 10%, 5%, or 1% significance level under a random permutation test.

In one embodiment, the markers employed for providing a prognosis are selected such that the peripheral blood expression profile of each marker is correlated with a class distinction under a class-based correlation analysis (such as the nearest-neighbor analysis), where the class distinction represents an idealized expression pattern of the selected genes in tissue samples, such as peripheral blood samples, of asthma patients who have different assessments. In many cases, the selected markers are correlated with the class distinction at above the 50%, 25%, 10%, 5%, or 1% significance level under a random permutation test.

The markers can also be selected such that the average expression profile of each marker in tissue samples, such as peripheral blood samples, of one class of asthma patients is statistically different from that in another class of asthma patients. For instance, the p-value under a Student's t-test for the observed difference can be no more than 0.05, 0.01, 0.005, 0.001, or less. In addition, the markers can be selected such that the average expression level of each marker in one class of patients is at least 2-, 3-, 4-, 5-, 10-, or 20-fold different from that in another class of patients.

The expression profile of a patient of interest can be compared to one or more reference expression profiles. The reference expression profiles can be determined concurrently with the expression profile of the patient of interest. The reference expression profiles can also be predetermined or prerecorded in electronic or other types of storage media.

The reference expression profiles can include average expression profiles, or individual profiles representing gene expression patterns in particular patients. In one embodiment, the reference expression profiles used for a diagnosis of asthma include an average expression profile of the marker(s) in tissue samples, such as peripheral blood samples, of healthy volunteers. In one embodiment, the reference expression profiles include an average expression profile of the marker(s) in tissue samples, such as peripheral blood samples, of reference asthma patients who have known or determinable disease status. Any averaging method may be used, such as arithmetic means, harmonic means, average of absolute values, average of log-transformed values, or weighted average. In one example, the reference asthma patients have the same disease assessment. In another example, the reference patients can are healthy volunteers used in a diagnostic method. In another example, the reference asthma patients can be divided into at least two classes, each class of patients having a different respective disease assessment. The average expression profile in each class of patients constitutes a separate reference expression profile, and the expression profile of the patient of interest is compared to each of these reference expression profiles.

In another embodiment, the reference expression profiles include a plurality of expression profiles, each of which represents the expression pattern of the marker(s) in a particular asthma patient. Other types of reference expression profiles can also be used in the present invention. In yet another embodiment, the present invention uses a numerical threshold as a control level. The numerical threshold may comprise a ratio, including, but not limited to, the ratio of the expression level of a marker in an asthma patient in relation to the expression level of the same marker in a healthy volunteer; or the ratio between the expression levels of the marker in an asthma patient both before and after treatment. The numerical threshold may also by a ratio of marker expression levels between patients with differing disease assessments.

In another embodiment, the absolute expression level(s) of the marker(s) are detected or measured and compared to reference expression level(s) for the purposes of providing a diagnosis or aiding in the selection of a treatment. The reference expression level is obtained from a control sample in this embodiment, the control sample being derived from either a healthy individual or an asthma patient prior to treatment.

The expression profile of the patient of interest and the reference expression profile(s) can be constructed in any form. In one embodiment, the expression profiles comprise the expression level of each marker used in outcome prediction. The expression levels can be absolute, normalized, or relative levels. Suitable normalization procedures include, but are not limited to, those used in nucleic acid array gene expression analyses or those described in Hill, et al., (Hill (2001) Genome Biol. 2:research0055.1-0055.13). In one example, the expression levels are normalized such that the mean is zero and the standard deviation is one. In another example, the expression levels are normalized based on internal or external controls, as appreciated by those skilled in the art. In still another example, the expression levels are normalized against one or more control transcripts with known abundances in blood samples. In many cases, the expression profile of the patient of interest and the reference expression profile(s) are constructed using the same or comparable methodologies.

In another embodiment, each expression profile being compared comprises one or more ratios between the expression levels of different markers. An expression profile can also include other measures that are capable of representing gene expression patterns.

The peripheral blood samples used in the present invention can be either whole blood samples, or samples comprising enriched PBMCs. In one example, the peripheral blood samples used for preparing the reference expression profile(s) comprise enriched or purified PBMCs, and the peripheral blood sample used for preparing the expression profile of the patient of interest is a whole blood sample. In another example, all of the peripheral blood samples employed in outcome prediction comprise enriched or purified PBMCs. In many cases, the peripheral blood samples are prepared from the patient of interest and reference patients using the same or comparable procedures.

Other types of blood samples can also be employed in the present invention, and the gene expression profiles in these blood samples are statistically significantly correlated with patient outcome.

The peripheral blood samples used in the present invention can be isolated from respective patients at any disease or treatment stage, and the correlation between the gene expression patterns in these peripheral blood samples, the health status, or clinical outcome is statistically significant. In many embodiments, the health status is measured by a comparison of the patient's expression profile or absolute marker(s) expression level(s) as compared to an absolute level of a marker in one or more healthy volunteers or an averaged or correlated expression profile from two or more healthy volunteers. In many embodiments, clinical outcome is measured by patients' response to a therapeutic treatment, and all of the blood samples used in outcome prediction are isolated prior to the therapeutic treatment. The expression profiles derived from the blood samples are therefore baseline expression profiles for the therapeutic treatment.

Construction of the expression profiles typically involves detection of the expression level of each marker used in the health status determination or outcome prediction. Numerous methods are available for this purpose. For instance, the expression level of a gene can be determined by measuring the level of the RNA transcript(s) of the gene(s). Suitable methods include, but are not limited to, quantitative RT-PCR, Northern blot, in situ hybridization, slot-blotting, nuclease protection assay, and nucleic acid array (including bead array). The expression level of a gene can also be determined by measuring the level of the polypeptide(s) encoded by the gene. Suitable methods include, but are not limited to, immunoassays (such as ELISA, RIA, FACS, or Western blot), 2-dimensional gel electrophoresis, mass spectrometry, or protein arrays.

In one aspect, the expression level of a marker is determined by measuring the RNA transcript level of the gene in a tissue sample, such as a peripheral blood sample. RNA can be isolated from the peripheral blood or tissue sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR (RT-PCR), isothermal amplification, ligase chain reaction, and Qbeta replicase.

In one embodiment, the amplification protocol employs reverse transcriptase. The isolated mRNA can be reverse transcribed into cDNA using a reverse transcriptase, and a primer consisting of oligo (dT) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TaqMan, ABI) is used for detecting or comparing the RNA transcript level of a marker of interest. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a marker of interest. The nucleic acid arrays can be commercial oligonucleotide or cDNA arrays. They can also be custom arrays comprising concentrated probes for the markers of the present invention. In many examples, at least 15%, 20%, 25%, 30%, 35%, 40%, 45%, 50%, or more of the total probes on a custom array of the present invention are probes for asthma markers. These probes can hybridize under stringent or nucleic acid array hybridization conditions to the RNA transcripts, or the complements thereof, of the corresponding markers.

As used herein, “stringent conditions” are at least as stringent as, for example, conditions G-L shown in Table 3. “Highly stringent conditions” are at least as stringent as conditions A-F shown in Table 3. Hybridization is carried out under the hybridization conditions (Hybridization Temperature and Buffer) for about four hours, followed by two 20-minute washes under the corresponding wash conditions (Wash Temp. and Buffer).

In one example, a nucleic acid array of the present invention includes at least 2, 5, 10, or more different probes. Each of these probes is capable of hybridizing under stringent or nucleic acid array hybridization conditions to a different respective marker of the present invention. Multiple probes for the same marker can be used on the same nucleic acid array. The probe density on the array can be in any range.

The probes for a marker of the present invention can be a nucleic acid probe, such as, DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the markers can be stably attached to discrete regions on a nucleic acid array. By “stably attached,” it means that a probe maintains its position relative to the attached discrete region during hybridization and signal detection. The position of each discrete region on the nucleic acid array can be either known or determinable. All of the methods known in the art can be used to make the nucleic acid arrays of the present invention.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

Hybridization probes or amplification primers for the markers of the present invention can be prepared by using any method known in the art.

In one embodiment, the probes/primers for a marker significantly diverge from the sequences of other markers. This can be achieved by checking potential probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by those skilled in the art.

In another embodiment, the probes for markers can be polypeptide in nature, such as, antibody probes. The expression levels of the markers of the present invention are thus determined by measuring the levels of polypeptides encoded by the markers. Methods suitable for this purpose include, but are not limited to, immunoassays such as ELISA, RIA, FACS, dot blot, Western Blot, immunohistochemistry, and antibody-based radio-imaging. In addition, high-throughput protein sequencing, 2-dimensional SDS-polyacrylamide gel electrophoresis, mass spectrometry, or protein arrays can be used.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can be detected. Detection can be achieved by the addition of a second antibody which is specific for the target proteins and is linked to a detectable label. Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying ELISA, the samples suspected of containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, ¹²⁵I. In one embodiment, a fixed concentration of ¹²⁵I-labeled polypeptide is incubated with a series of dilution of an antibody specific to the polypeptide. When the unlabeled polypeptide is added to the system, the amount of the ¹²⁵I-polypeptide that binds to the antibody is decreased. A standard curve can therefore be constructed to represent the amount of antibody-bound ¹²⁵I-polypeptide as a function of the concentration of the unlabeled polypeptide. From this standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing these antibodies are well known in the art. In one embodiment, the antibodies of the present invention can bind to the corresponding marker gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the markers. Methods for making protein arrays or biochips are well known in the art. In many embodiments, a substantial portion of probes on a protein array of the present invention are antibodies specific for the marker products. For instance, at least 10%, 20%, 30%, 40%, 50%, or more probes on the protein array can be antibodies specific for the marker gene products.

In yet another aspect, the expression levels of the markers are determined by measuring the biological functions or activities of these genes. Where a biological function or activity of a gene is known, suitable in vitro or in vivo assays can be developed to evaluate the function or activity. These assays can be subsequently used to assess the level of expression of the marker.

After the expression level of each marker is determined, numerous approaches can be employed to compare expression profiles. Comparison of the expression profile of a patient of interest to the reference expression profile(s) can be conducted manually or electronically. In one example, comparison is carried out by comparing each component in one expression profile to the corresponding component in a reference expression profile. The component can be the expression level of a marker, a ratio between the expression levels of two markers, or another measure capable of representing gene expression patterns. The expression level of a gene can have an absolute or a normalized or relative value. The difference between two corresponding components can be assessed by fold changes, absolute differences, or other suitable means.

Comparison of the expression profile of a patient of interest to the reference expression profile(s) can also be conducted using pattern recognition or comparison programs, such as the k-nearest-neighbors algorithm as described in Armstrong, et al., (Armstrong (2002) Nature Genetics 30:41-47), or the weighted voting algorithm as described below. In addition, the serial analysis of gene expression (SAGE) technology, the GEMTOOLS gene expression analysis program (Incyte Pharmaceuticals), the GeneCalling and Quantitative Expression Analysis technology (Curagen), and other suitable methods, programs or systems can be used to compare expression profiles.

Multiple markers can be used in the comparison of expression profiles. For instance, 2, 4, 6, 8, 10, 12, 14, or more markers can be used. In addition, the marker(s) used in the comparison can be selected to have relatively small p-values (e.g., two-sided p-values). In many examples, the p-values indicate the statistical significance of the difference between gene expression levels in different classes of patients. In many other examples, the p-values suggest the statistical significance of the correlation between gene expression patterns and clinical outcome. In one embodiment, the markers used in the comparison have p-values of no greater than 0.05, 0.01, 0.001, 0.0005, 0.0001, or less. Markers with p-values of greater than 0.05 can also be used. These genes may be identified, for instance, by using a relatively small number of blood samples.

Similarity or difference between the expression profile of a patient of interest and a reference expression profile is indicative of the class membership of the patient of interest. Similarity or difference can be determined by any suitable means. The comparison can be qualitative, quantitative, or both.

In one example, a component in a reference profile is a mean value, and the corresponding component in the expression profile of the patient of interest falls within the standard deviation of the mean value. In such a case, the expression profile of the patient of interest may be considered similar to the reference profile with respect to that particular component. Other criteria, such as a multiple or fraction of the standard deviation or a certain degree of percentage increase or decrease, can be used to measure similarity.

In another example, at least 50% (e.g., at least 60%, 70%, 80%, 90%, or more) of the components in the expression profile of the patient of interest are considered similar to the corresponding components in a reference profile. Under these circumstances, the expression profile of the patient of interest may be considered similar to the reference profile. Different components in the expression profile may have different weights for the comparison. In some cases, lower percentage thresholds (e.g., less than 50% of the total components) are used to determine similarity.

The marker(s) and the similarity criteria can be selected such that the accuracy of the diagnostic determination or the outcome prediction (the ratio of correct calls over the total of correct and incorrect calls) is relatively high. For instance, the accuracy of the determination or prediction can be at least 50%, 60%, 70%, 80%, 90%, or more.

The effectiveness of treatment prediction can also be assessed by sensitivity and specificity. The markers and the comparison criteria can be selected such that both the sensitivity and specificity of outcome prediction are relatively high. For instance, the sensitivity and specificity can be at least 50%, 60%, 70%, 80%, 90%, 95%, or more. As used herein, “sensitivity” refers to the ratio of correct positive calls over the total of true positive calls plus false negative calls, and “specificity” refers to the ratio of correct negative calls over the total of true negative calls plus false positive calls.

Moreover, peripheral blood expression profile-based health status determination or outcome prediction can be combined with other clinical evidence to aid in treatment selection, improve the effectiveness of treatment, or accuracy of outcome prediction.

In many embodiments, the expression profile of a patient of interest is compared to at least two reference expression profiles. Each reference expression profile can include an average expression profile, or a set of individual expression profiles each of which represents the gene expression pattern in a particular asthma patient or disease-free human. Suitable methods for comparing one expression profile to two or more reference expression profiles include, but are not limited to, the weighted voting algorithm or the k-nearest-neighbors algorithm. Softwares capable of performing these algorithms include, but are not limited to, GeneCluster 2 software. GeneCluster2 software is available from MIT Center for Genome Research at Whitehead Institute. Both the weighted voting and k-nearest-neighbors algorithms employ gene classifiers that can effectively assign a patient of interest to a health status, outcome or effectiveness of treatment class. By “effectively,” it means that the class assignment is statistically significant. In one example, the effectiveness of class assignment is evaluated by leave-one-out cross validation or k-fold cross validation. The prediction accuracy under these cross validation methods can be, for instance, at least 50%, 60%, 70%, 80%, 90%, 95%, or more. The prediction sensitivity or specificity under these cross validation methods can also be at least 50%, 60%, 70%, 80%, 90%, 95%, or more. Markers or class predictors with low assignment sensitivity/specificity or low cross validation accuracy, such as less than 50%, can also be used in the present invention.

Under one version of the weighted voting algorithm, each gene in a class predictor casts a weighted vote for one of the two classes (class 0 and class 1). The vote of gene “g” can be defined as v_{g}=a_{g} (x_{g}−b_{g}), wherein a_{g} equals to P(g,c) and reflects the correlation between the expression level of gene “g” and the class distinction between the two classes, b_{g} is calculated as b_{g}=[x0(g)+x1(g)]/2 and represents the average of the mean logs of the expression levels of gene “g” in class 0 and class 1, and x_{g} is the normalized log of the expression level of gene “g” in the sample of interest. A positive v_{g} indicates a vote for class 0, and a negative v_{g} indicates a vote for class 1. V0 denotes the sum of all positive votes, and V1 denotes the absolute value of the sum of all negative votes. A prediction strength PS is defined as PS=(V0−V1)/(V0+V1). Thus, the prediction strength varies between −1 and 1 and can indicate the support for one class (e.g., positive PS) or the other (e.g., negative PS). A prediction strength near “0” suggests narrow margin of victory, and a prediction strength close to “1” or “−1” indicates wide margin of victory. See Slonim, et al., (Slonim (2000) Procs. of the Fourth Annual International Conference on Computational Molecular Biology Tokyo, Japan, April 8-11, p 263-272); and Golub, et al. (Golub (1999) Science 286: 531-537).

Suitable prediction strength (PS) thresholds can be assessed by plotting the cumulative cross-validation error rate against the prediction strength. In one embodiment, a positive predication is made if the absolute value of PS for the sample of interest is no less than 0.3. Other PS thresholds, such as no less than 0.1, 0.2, 0.4 or 0.5, can also be selected for class prediction. In many embodiments, a threshold is selected such that the accuracy of prediction is optimized and the incidence of both false positive and false negative results is minimized.

Any class predictor constructed according to the present invention can be used for the class assignment of an asthma patient of interest. In many examples, a class predictor employed in the present invention includes n markers identified by the neighborhood analysis, where n is an integer greater than 1.

The expression profile of a patient of interest can also be compared to two or more reference expression profiles by other means. For instance, the reference expression profiles can include an average peripheral blood expression profile for each class of patients. The fact that the expression profile of a patient of interest is more similar to one reference profile than to another suggests that the patient of interest is more likely to have the clinical outcome associated with the former reference profile than that associated with the latter reference profile.

In another embodiment, average expression profiles can be compared to each other as well as to a reference expression profile. In one embodiment, an expression profile of a patient is compared to a reference expression profile derived from a healthy volunteer or healthy volunteers, and is also compared to an expression profile of an asthma patient or patients to make a diagnosis. In another embodiment, an expression profile of an asthma patient before treatment is compared to a reference expression profile, and is also compared to an expression profile of the same asthma patient after treatment to determine the effectiveness of the treatment. In another embodiment, the expression profiles of the patient both before and after treatment are compared to a reference expression profile, as well as to each other.

In one particular embodiment, the present invention features diagnosis of a patient of interest. Patients can be divided into two classes based on their over- and/or under-expression of asthma markers of interest. One class of patients is diagnosed as having asthma (asthmatics) and the other does not (healthy volunteers). Asthma markers that are correlated with a class distinction between those two classes of patients can be identified and then used to assign the patient of interest to one of these two health status classes, thus rendering a diagnosis. Examples of asthma markers suitable for this purpose are depicted in Tables 6, 7a, 7b, 8a, or 8b. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051.

In one particular embodiment, the present invention features prediction of clinical outcome or prognosis of an asthma patient of interest. Asthma patients can be divided into at least two classes based on their responses to a specified treatment regimen. One class of patients (responders) has complete relief of symptoms in response to the treatment, and the other class of patients (non-responders) has neither complete relief from the symptoms of pulmonary obstruction nor partial relief in response to the treatment. Asthma markers that are correlated with a class distinction between those two classes of patients can be identified and then used to assign the patient of interest to one of these two outcome classes. Examples of asthma markers suitable for this purpose are depicted in Tables 6, 7a, 7b, 8a, or 8b. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051.

The present invention also provides for a method for selecting a treatment or treatment regime involving the use of one or more of the markers of the invention in the diagnosis of the patient as previously described. In a particular embodiment, the expression level of one or more markers of the present invention can be detected and compared to a reference expression level with the subsequent diagnosis of the patient as having asthma should the comparison indicate as such. If the patient is diagnosed as having asthma, treatments or treatment regimes known in the art may be applied in conjunction with this method. Diagnosis of the patient may be determined using any and all of the methods described relating to comparative and statistical methods, techniques, and analyses of marker expression levels, as well as any and all such comparative and statistical methods, techniques, and analyses known to, and commonly used by, one skilled in the art of pharmacogenomics.

In one example, the treatment or treatment regime includes the administration of at least one therapeutic selected from the group including, but not limited to, an anti-histamine, a steroid, an immunomodulator, an IgE downregulator, an immunosuppressant, a bronchodilator/beta-2 agonist, an adenosine A2a receptor agonist, a leukotriene antagonist, a thromboxane A2 synthesis inhibitor, a 5-lipoxygenase inhibitor, an anti-cholinergic, a LTB-4 antagonist, a K+ channel opener, a VLA-4 antagonist, a neurokine antagonist, theophylline, a thromboxane A2 receptor antagonist, a beta-2 adrenoceptor agonist, a soluble interleukin receptor, a 5-lipoxygenase activating protein inhibitor, an arachidonic acid antagonist, an anti-inflammatory, a membrane channel inhibitor, an anti-interleukin antibody, a PDE-4 inhibitor, and a protease inhibitor. Treatments or treatment regimes may also include, but are not limited to, drug therapy, including any and all treatments/therapeutics exemplified in Tables 1 and 2, gene therapy, immunotherapy, radiation therapy, biological therapy, and surgery, as well as any and all other therapeutic methods and treatments known to, and commonly used by, the skilled artisan.

Markers or class predictors capable of distinguishing three or more outcome classes can also be employed in the present invention. These markers can be identified using multi-class correlation metrics. Suitable programs for carrying out multi-class correlation analysis include, but are not limited to, GeneCluster 2 software (MIT Center for Genome Research at Whitehead Institute, Cambridge, Mass.). Under the analysis, patients having asthma are divided into at least three classes, and each class of patients has a different respective clinical outcome. The markers identified under multi-class correlation analysis are differentially expressed in one embodiment in PBMCs of one class of patients relative to PBMCs of other classes of patients. In one embodiment, the identified markers are correlated with a class distinction at above the 1%, 5%, 10%, 25%, or 50% significance level under a permutation test. The class distinction in this embodiment represents an idealized expression pattern of the identified genes in peripheral blood samples of patients who have different clinical outcomes.

### Gene Expression Analysis

The relationship between tissue gene expression profiles, especially peripheral blood gene expression profiles, and diagnosis, prognosis, treatment selection, or treatment effectiveness can be evaluated by using global gene expression analyses. Methods suitable for this purpose include, but are not limited to, nucleic acid arrays (such as cDNA or oligonucleotide arrays), 2-dimensional SDS-polyacrylamide gel electrophoresis/mass spectrometry, and other high throughput nucleotide or polypeptide detection techniques.

Nucleic acid arrays allow for quantitative detection of the expression of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,228,220, and 6,391,562.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical, or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides derived from one sample, such as PBMCs from a patient in a selected health status or outcome class, are hybridized to the probes on a nucleic acid array. Signals detected after the formation of hybridization complexes correlate to the polynucleotide levels in the sample. In the differential hybridization format, polynucleotides derived from two biological samples, such as one from a patient in a first status or outcome class and the other from a patient in a second status or outcome class, are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from a nucleic acid array can be analyzed using commercially available software, such as those provided by Affymetrix or Agilent Technologies. Controls, such as for scan sensitivity, probe labeling, and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of genes are normalized across the samples such that the mean is zero and the standard deviation is one. In another embodiment, the expression data detected by nucleic acid arrays are subject to a variation filter that excludes genes showing minimal or insignificant variation across all samples.

### Correlation Analysis

The gene expression data collected from nucleic acid arrays can be correlated with diagnosis, clinical outcome, treatment selection, or treatment effectiveness using a variety of methods. Methods suitable for this purpose include, but are not limited to, statistical methods (such as Spearman's rank correlation, Cox proportional hazard regression model, ANOVA/t test, or other rank tests or survival models) and class-based correlation metrics (such as nearest-neighbor analysis).

In one embodiment, patients with asthma are divided into at least two classes based on their responses to a therapeutic treatment. In another embodiment, a patient of interest can be determined to belong to one of two classes based on the patient's health status. The correlation between peripheral blood gene expression (e.g., PBMC gene expression) and the health status, patient outcome or treatment effectiveness classes is then analyzed by a supervised cluster or learning algorithm. Supervised algorithms suitable for this purpose include, but are not limited to, nearest-neighbor analysis, support vector machines, the SAM method, artificial neural networks, and SPLASH. Under a supervised analysis, health status or clinical outcome of, or treatment effectiveness for, each patient is either known or determinable. Genes that are differentially expressed in peripheral blood cells (e.g., PBMCs) of one class of patients relative to another class of patients can be identified. These genes can be used as surrogate markers for predicting/determining health status or clinical outcome of, or treatment effectiveness for, an asthma patient of interest. Many of the genes thus identified are correlated with a class distinction that represents an idealized expression pattern of these genes in patients of different health status, outcome, or treatment effectiveness classes.

In another embodiment, patients with asthma can be divided into at least two classes based on their peripheral blood gene expression profiles. Methods suitable for this purpose include unsupervised clustering algorithms, such as self-organized maps (SOMs), k-means, principal component analysis, and hierarchical clustering. A substantial number (e.g., at least 50%, 60%, 70%, 80%, 90%, or more) of patients in one class may have a first health status, clinical outcome, or treatment effectiveness profile, and a substantial number of patient in another class my have a second health status, clinical outcome, or treatment effectiveness profile. Genes that are differentially expressed in the peripheral blood cells of one class of patients relative to another class of patients can be identified. These genes can also be used as markers for predicting/determining health status, clinical outcome of, or treatment effectiveness for, an asthma patient of interest.

In yet another embodiment, patients with asthma can be divided into three or more classes based on their clinical outcomes or peripheral blood gene expression profiles. Multi-class correlation metrics can be employed to identify genes that are differentially expressed in one class of patients relative to another class. Exemplary multi-class correlation metrics include, but are not limited to, those employed by GeneCluster 2 software provided by MIT Center for Genome Research at Whitehead Institute (Cambridge, Mass.).

In a further embodiment, nearest-neighbor analysis (also known as neighborhood analysis) is used to correlate peripheral blood gene expression profiles with health status, clinical outcome of, or treatment effectiveness for, asthma patients. The algorithm for neighborhood analysis is described in Slonim, et al., (Slonim (2000) Procs. of the Fourth Annual International Conference on Computational Molecular Biology Tokyo, Japan, April 8-11, p 263-272); and Golub, et al. (Golub (1999) Science 286: 531-537); and U.S. Pat. No. 6,647,341. Under one version of the neighborhood analysis, the expression profile of each gene can be represented by an expression vector g=(e₁, e₂, e₃, . . . , eₙ), where eᵢ corresponds to the expression level of gene “g” in the ith sample. A class distinction can be represented by an idealized expression pattern c=(c₁, c₂, c₃, . . . , cₙ), where cᵢ=1 or −1, depending on whether the ith sample is isolated from class 0 or class 1. Class 0 may include patients having a first health status, clinical outcome, or treatment effectiveness profile, and class 1 includes patients having a second health status, clinical outcome, or treatment effectiveness profile. Other forms of class distinction can also be employed. Typically, a class distinction represents an idealized expression pattern, where the expression level of a gene is uniformly high for samples in one class and uniformly low for samples in the other class.

The correlation between “g” and the class distinction can be measured by a signal-to-noise score:

P(g,c)=[μ₁(g)−μ₂(g)]/[σ₁(g)+σ₂(g)]
where μ₁(g) and μ₂(g) represent the means of the log-transformed expression levels of gene “g” in class 0 and class 1, respectively, and σ₁(g) and σ₂(g) represent the standard deviation of the log-transformed expression levels of gene “g” in class 0 and class 1, respectively. A higher absolute value of a signal-to-noise score indicates that the gene is more highly expressed in one class than in the other. In one example, the samples used to derive the signal-to-noise scores comprise enriched or purified PBMCs and, therefore, the signal-to-noise score P(g,c) represents the correlation between the class distinction and the expression level of gene “g” in PBMCs.

The correlation between gene “g” and the class distinction can also be measured by other methods, such as by the Pearson correlation coefficient or the Euclidean distance, as appreciated by those skilled in the art.

The significance of the correlation between marker expression profiles and the class distinction is evaluated using a random permutation test. An unusually high density of genes within the neighborhoods of the class distinction, as compared to random patterns, suggests that many genes have expression patterns that are significantly correlated with the class distinction. The correlation between genes and the class distinction can be diagrammatically viewed through a neighborhood analysis plot, in which the y-axis represents the number of genes within various neighborhoods around the class distinction and the x-axis indicates the size of the neighborhood (i.e., P(g,c)). Curves showing different significance levels for the number of genes within corresponding neighborhoods of randomly permuted class distinctions can also be included in the plot.

In many embodiments, the markers employed in the present invention are above the median significance level in the neighborhood analysis plot. This means that the correlation measure P(g,c) for each marker is such that the number of genes within the neighborhood of the class distinction having the size of P(g,c) is greater than the number of genes within the corresponding neighborhoods of random permuted class distinctions at the median significance level. In many other embodiments, the markers employed in the present invention are above the 40%, 30%, 20%, 10%, 5%, 2%, or 1% significance level. As used herein, x % significance level means that x % of random neighborhoods contain as many genes as the real neighborhood around the class distinction.

In another aspect, the correlation between marker expression profiles and health status or clinical outcome can be evaluated by statistical methods. One exemplary statistical method employs Spearman's rank correlation coefficient, which has the formula of:

rₛ=SS_{UV}/(SS_{UU}SS_{VV})^{1/2}
where SS_{UV}=ΣUᵢVᵢ−[(ΣUᵢ)(ΣVᵢ)]/n, SS_{UU}=ΣVᵢ²−[(ΣVᵢ)²]/n, and SS_{VV}=ΣUᵢ²−[(ΣUᵢ)²]/n. Uᵢ is the expression level ranking of a gene of interest, Vᵢ is the ranking of the health status or clinical outcome, and n represents the number of patients. The shortcut formula for Spearman's rank correlation coefficient is rₛ=1−(6×Σdᵢ²)/[n(n²−1)], where dᵢ=Uᵢ−Vᵢ. The Spearman's rank correlation is similar to the Pearson's correlation except that it is based on ranks and is thus more suitable for data that is not normally distributed. See, for example, Snedecor and Cochran (Snedecor (1989) Statistical Methods, 8th edition, Iowa State University Press, Ames, Iowa). The correlation coefficient is tested to assess whether it differs significantly from a value of 0 (i.e., no correlation).

The correlation coefficients for each marker identified by the Spearman's rank correlation can be either positive or negative, provided that the correlation is statistically significant. In many embodiments, the p-value for each marker thus identified is no more than 0.05, 0.01, 0.005, 0.001, 0.0005, 0.0001, or less. In many other embodiments, the Spearman correlation coefficients of the markers thus identified have absolute values of at least 0.3, 0.4, 0.5, 0.6, 0.7, 0.8, 0.9, or more.

Another exemplary statistical method is Cox proportional hazard regression model, which has the formula of:

log hᵢ(t)=α(t)+βⱼxᵢⱼ
wherein hᵢ(t) is the hazard function that assesses the instantaneous risk of demise at time t, conditional on survival to that time, α(t) is the baseline hazard function, and xᵢⱼ is a covariate which may represent, for example, the expression level of marker j in a peripheral blood sample or other tissue sample. (See Cox (1972) Journal of the Royal Statistical Society, Series B 34:187) Additional covariates, such as interactions between covariates, can also be included in Cox proportional hazard model. As used herein, the terms “demise” or “survival” are not limited to real death or survival. Instead, these terms should be interpreted broadly to cover any type of time-associated events. In many cases, the p-values for the correlation under Cox proportional hazard regression model are no more than 0.05, 0.01, 0.005, 0.001, 0.0005, 0.0001, or less. The p-values for the markers identified under Cox proportional hazard regression model can be determined by the likelihood ratio test, Wald test, the Score test, or the log-rank test. In one embodiment, the hazard ratios for the markers thus identified are at least 1.5, 2, 3, 4, 5, or more. In another embodiment, the hazard ratios for the markers thus identified are no more than 0.67, 0.5., 0.33, 0.25., 0.2, or less.

Other rank tests, scores, measurements, or models can also be employed to identify markers whose expression profiles in peripheral blood samples, or other tissue samples, are correlated with clinical outcome of asthma. These tests, scores, measurements, or models can be either parametric or nonparametric, and the regression may be either linear or non-linear. Many statistical methods and correlation/regression models can be carried out using commercially available programs.

Class predictors can be constructed using the markers of the present invention. These class predictors can be used to assign an asthma patient of interest to a health status, outcome, or treatment effectiveness class. In one embodiment, the markers employed in a class predictor are limited to those shown to be significantly correlated with a class distinction by the permutation test, such as those at or above the 1%, 2%, 5%, 10%, 20%, 30%, 40%, or 50% significance level. In another embodiment, the PBMC expression level of each marker in a class predictor is substantially higher or substantially lower in one class of patients than in another class of patients. In still another embodiment, the markers in a class predictor have top absolute values of P(g,c). In yet another embodiment, the p-value under a Student's t-test (e.g., two-tailed distribution, two sample unequal variance) for each marker in a class predictor is no more than 0.05, 0.01, 0.005, 0.001, 0.0005, 0.0001, or less. For each marker, the p-value suggests the statistical significance of the difference observed between the average PBMC, or other tissue, expression profiles of the gene in one class of patients versus another class of patients. Lesser p-values indicate more statistical significance for the differences observed between the different classes of asthma patients.

The SAM method can also be used to correlate peripheral blood gene expression profiles with different health status, outcome, or treatment effectiveness classes. The prediction analysis of microarrays (PAM) method can then be used to identify class predictors that can best characterize a predefined health status, outcome or treatment effectiveness class and predict the class membership of new samples. See Tibshirani, et al., (Tibshirani (2002) Proc. Natl. Acad. Sci. U.S.A. 99:6567-6572).

In many embodiments, a class predictor of the present invention has high prediction accuracy under leave-one-out cross validation, 10-fold cross validation, or 4-fold cross validation. For instance, a class predictor of the present invention can have at least 50%, 60%, 70%, 80%, 90%, 95%, or 99% accuracy under leave-one-out cross validation, 10-fold cross validation, or 4-fold cross validation. In a typical k-fold cross validation, the data is divided into k subsets of approximately equal size. The model is trained k times, each time leaving out one of the subsets from training and using the omitted subset as the test sample to calculate the prediction error. If k equals the sample size, it becomes the leave-one-out cross validation.

Other class-based correlation metrics or statistical methods can also be used to identify markers whose expression profiles in peripheral blood samples, or other tissue samples, are correlated with health status or clinical outcome of asthma patients. Many of these methods can be performed by using commercial or publicly accessible software packages.

Other methods capable of identifying asthma markers include, but are not limited to, RT-PCR, Northern blot, in situ hybridization, and immunoassays such as ELISA, RIA, or Western blot. These genes are differentially expressed in peripheral blood cells (e.g., PBMCs), or other tissues, of one class of patients relative to another class of patients. In many cases, the average marker expression level of each of these genes in one class of patients is statistically different from that in another class of patients. For instance, the p-value under an appropriate statistical significance test (e.g., Student's t-test) for the observed difference can be no more than 0.05, 0.01, 0.005, 0.001, 0.0005, 0.0001, or less. In many other cases, each marker thus identified has at least 2-, 3-, 4-, 5-, 10-, or 20-fold difference in the average PBMC, or other tissue, expression level between one class of patients and another class of patients.

### Asthma Treatment

Any asthma treatment regime, and its effectiveness, can be analyzed according to the present invention. Example of these asthma treatments include, but are not limited to, drug therapy, gene therapy, radiation therapy, immunotherapy, biological therapy, surgery, or a combination thereof. Other conventional, non-conventional, novel, or experimental therapies, including treatments under clinical trials, can also be evaluated according to the present invention.

A variety of anti-asthma agents can be used to treat asthma. An “asthma/allergy medicament” as used herein is a composition of matter which reduces the symptoms, inhibits the asthmatic or allergic reaction, or prevents the development of an allergic or asthmatic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below. Examples of useful medicaments according to the present invention that are either on the market or in development are presented in Tables 1 and 2.

In most embodiments the asthma/allergy medicament is useful to some degree for treating both asthma and allergy. These are referred to as asthma medicaments. Asthma medicaments include, but are not limited, PDE-4 inhibitors, bronchodilator/beta-2 agonists, beta-2 adrenoreceptor ant/agonists, anticholinergics, steroids, K⁺ channel openers, VLA-4 antagonists, neurokin antagonists, thromboxane A2 synthesis inhibitors, xanthines, arachidonic acid antagonists, 5 lipoxygenase inhibitors, thromboxin A2 receptor antagonists, thromboxane A2 antagonists, inhibitor of 5-lipox activation proteins, and protease inhibitors.

Bronchodilator/beta-2 agonists are a class of compounds which cause bronchodilation or smooth muscle relaxation. Bronchodilator/beta-2 agonists include, but are not limited to, salmeterol, salbutamol, albuterol, terbutaline, D2522/formoterol, fenoterol, bitolterol, pirbuerol, methylxanthines and orciprenaline. Long-acting beta-2 agonists and bronchodilators are compounds which are used for long-term prevention of symptoms in addition to the anti-inflammatory therapies. They function by causing bronchodilation, or smooth muscle relaxation, following adenylate cyclase activation and increase in cyclic AMP producing functional antagonism of bronchoconstriction. These compounds also inhibit mast cell mediator release, decrease vascular permeability and increase mucociliary clearance. Long-acting beta-2 agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. It is also believed that these compounds may effect eosinophilic infiltration into bronchial mucosa and decrease T-lymphocyte numbers in the epithelium. Dose-related acute toxicities are a particular problem with these types of compounds. As a result, routine serum concentration should be monitored in order to account for the toxicity and narrow therapeutic range arising from individual differences in metabolic clearance. Side effects include tachycardia, nausea and vomiting, tachyarrhythmias, central nervous system stimulation, headache, seizures, hematemesis, hyperglycemia and hypokalemia. Short-acting beta-2 agonists/bronchodilators relax airway smooth muscle, causing the increase in air flow. These types of compounds are a preferred drug for the treatment of acute asthmatic systems. Previously, short-acting beta-2 agonists had been prescribed on a regularly-scheduled basis in order to improve overall asthma symptoms. Later reports, however, suggested that regular use of this class of drugs produced significant diminution in asthma control and pulmonary function (Sears (1990) Lancet 336:1391-6). Other studies showed that regular use of some types of beta-2 agonists produced no harmful effects over a four-month period but also produced no demonstrable effects (Drazen (1996) N. Eng. J. Med. 335:841-7). As a result of these studies, the daily use of short-acting beta-2 agonists is not generally recommended. Short-acting beta-2 agonists include, but are not limited to, albuterol, bitolterol, pirbuterol, and terbutaline. Some of the adverse effects associated with the mastration of short-acting beta-2 agonists include tachycardia, skeletal muscle tremor, hypokalemia, increased lactic acid, headache, and hyperglycemia.

Other allergy medicaments are commonly used in the treatment of asthma. These include, but are not limited to, anti-histamines, steroids, and prostaglandin inducers. Anti-histamines are compounds which counteract histamine released by mast cells or basophils. Anti-histamines include, but are not limited to, loratidine, cetirizine, buclizine, ceterizine analogues, fexofenadine, terfenadine, desloratadine, norastemizole, epinastine, ebastine, astemizole, levocabastine, azelastine, tranilast, terfenadine, mizolastine, betatastine, CS 560, and HSR 609. Prostaglandins function by regulating smooth muscle relaxation. Prostaglandin inducers include, but are not limited to, S-575 1.

The steroids include, but are not limited to, beclomethasone, fluticasone, tramcinolone, budesonide, corticosteroids and budesonide. To date, the use of steroids in children has been limited by the observation that some steroid treatments have been reportedly associated with growth retardation. Therefore, caution should be observed in their use.

Corticosteroids are used long-term to prevent development of the symptoms, and suppress, control, and reverse inflammation arising from an initiator. Some corticosteroids can be administered by inhalation and others are administered systemically. The corticosteroids that are inhaled have an anti-inflammatory function by blocking late-reaction allergen and reducing airway hyper-responsiveness. These drugs also inhibit cytokine production, adhesion protein activation, and inflammatory cell migration and activation.

Corticosteroids include, but are not limited to, beclomethasome dipropionate, budesonide, flunisolide, fluticaosone, propionate, and triamcinoone acetonide. Although dexamethasone is a corticosteroid having anti-inflammatory action, it is not regularly used for the treatment of asthma/allergy in an inhaled form because it is highly absorbed and it has long-term suppressive side effects at an effective dose. Dexamethasone, however, can be administered at a low dose to reduce the side effects. Some of the side effects associated with corticosteroid include cough, dysphonia, oral thrush (candidiasis), and in higher doses, systemic effects, such as adrenal suppression, osteoporosis, growth suppression, skin thinning and easy bruising. (Barnes (1993) Am. J. Respir. Crit. Care Med. 153:1739-48)

Systemic corticosteroids include, but are not limited to, methylprednisolone, prednisolone and prednisone. Corticosteroids are used generally for moderate to severe exacerbations to prevent the progression, reverse inflammation and speed recovery. These anti-inflammatory compounds include, but are not limited to, methylprednisolone, prednisolone, and prednisone. Corticosteroids are associated with reversible abnormalities in glucose metabolism, increased appetite, fluid retention, weight gain, mood alteration, hypertension, peptic ulcer, and rarely asceptic necrosis of femur. These compounds are useful for short-term (3-10 days) prevention of the inflammatory reaction in inadequately controlled persistent asthma. They also function in a long-term prevention of symptoms in severe persistent asthma to suppress and control and actually reverse inflammation. The side effects associated with systemic corticosteroids are even greater than those associated with inhaled corticosteroids. Side effects include, for instance, reversible abnormalities in glucose metabolism, increased appetite, fluid retention, weight gain, mood alteration, hypertension, peptic ulcer and asceptic necrosis of femur, which are associated with short-term use. Some side effects associated with longer term use include adrenal axis suppression, growth suppression, dermal thinning, hypertension, diabetes, Cushing's syndrome, cataracts, muscle weakness, and in rare instances, impaired immune function. It is recommended that these types of compounds be used at their lowest effective dose (guidelines for the diagnosis and management of asthma; expert panel report to; NIH Publication No. 97-4051; July 1997). The inhaled corticosteroids are believed to function by blocking late reaction to allergen and reducing airway hyper-responsiveness. They are also believed to reverse beta-2-receptor downregulation and to inhibit microvascular leakage.

The immunomodulators include, but are not limited to, the group consisting of anti-inflammatory agents, leukotriene antagonists, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, eosinophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor mediated mechanisms. Substance P also has been shown to modulate distinct immediate hypersensitivity responses by stimulating the generation of arachidonic acid-derived mediators from mucosal mast cells. (J. McGillies (1987) Fed. Proc. 46:196-9) Substance P is a neuropeptide first identified in 1931 by Von Euler (Von Euler (1931) J. Physiol. (London) 72:74-87). Its amino acid sequence was reported by Chang (Chang (1971) Nature (London) 232:86-87). The immunoregulatory activity of fragments of substance P has been studied by Siemion (Siemion (1990) Molec. Immunol. 27:887-890).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules lack a binding strength to the receptor corresponding to the very strong interaction between the native IgE molecule and its receptor. The molecules having this strength tend to bind irreversibly to the receptor. However, such substances are relatively toxic since they can bind covalently and block other structurally similar molecules in the body. Of interest in this context is that the alpha chain of the IgE receptor belongs to a larger gene family of different IgG Fc receptors. These receptors are absolutely essential for the defense of the body against bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

These types of asthma/allergy medicaments are sometimes classified as long-term control medications or quick-relief medications. Long-term control medications include compounds such as corticosteroids (also referred to as glucocorticoids), methylprednisolone, prednisolone, prednisone, cromolyn sodium, nedocromil, long-acting beta-2-agonists, methylxanthines, and leukotriene modifiers. Quick relief medications are useful for providing quick relief of symptoms arising from allergic or asthmatic responses. Quick relief medications include short-acting beta-2 agonists, anticholinergics and systemic corticosteroids.

Chromolyn sodium and medocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from eosinophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratrapoium bromide. These compounds reverse only cholinerigically-mediated bronchospasm and do not modify any reaction to antigen. Side effects include drying of the mouth and respiratory secretions, increased wheezing in some individuals, blurred vision if sprayed in the eyes.

In addition to standard asthma/allergy medicaments other methods for treating asthma/allergy have been used either alone or in combination with established medicaments. One preferred, but frequently impossible, method of relieving allergies is allergen or initiator avoidance. Another method currently used for treating allergic disease involves the injection of increasing doses of allergen to induce tolerance to the allergen and to prevent further allergic reactions.

Allergen injection therapy (allergen immunotherapy) is known to reduce the severity of allergic rhinitis. This treatment has been theorized to involve the production of a different form of antibody, a protective antibody which is termed a “blocking antibody”. (Cooke (1935) Exp. Med. 62:733). Other attempts to treat allergy involve modifying the allergen chemically so that its ability to cause an immune response in the patient is unchanged, while its ability to cause an allergic reaction is substantially altered.

These methods, however, can take several years to be effective and are associated with the risk of side effects such as anaphylactic shock. The use of an immunostimulatory nucleic acid and asthma/allergy medicament in combination with an allergen avoids many of the side effects etc.

Commonly used allergy and asthma drugs which are currently in development or on the market are shown in Tables 1 and 2 respectively.

### Screening Methods

The invention also provides methods (also referred to herein as “screening assays”) for identifying agents capable of modulating marker expression (“modulators”), i.e., candidate or test compounds or agents comprising therapeutic moieties (e.g., peptides, peptidomimetics, peptoids, polynucleotides, small molecules or other drugs) which (a) bind to a marker gene product or (b) have a modulatory (e.g., upregulation or downregulation; stimulatory or inhibitory; potentiation/induction or suppression) effect on the activity of a marker gene product or, more specifically, (c) have a modulatory effect on the interactions of the marker gene product with one or more of its natural substrates, or (d) have a modulatory effect on the expression of the marker. Such assays typically comprise a reaction between the marker gene product and one or more assay components. The other components may be either the test compound itself, or a combination of test compound and a binding partner of the marker gene product.

The test compounds of the present invention are generally either small molecules or biomolecules. Small molecules include, but are not limited to, inorganic molecules and small organic molecules. Biomolecules include, but are not limited to, naturally-occurring and synthetic compounds that have a bioactivity in mammals, such as polypeptides, polysaccharides, and polynucleotides. In one embodiment, the test compound is a small molecule. In another embodiment, the test compound is a biomolecule. One skilled in the art will appreciate that the nature of the test compound may vary depending on the nature of the protein encoded by the marker of the present invention.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckerman et al. (Zuckerman (1994) J. Med. Chem. 37:2678-85); spatially addressable parallel solid phase or solution phase libraries; synthetic library methods requiring deconvolution; the “one-bead, one-compound” library method; and synthetic library methods using affinity chromatography selection. The biological library and peptoid library approaches are applicable to peptide, non-peptide oligomers or small molecule libraries of compound (Lam (1997) Anticancer Drug Des. 12:145).

The invention provides methods of screening test compounds for inhibitors of the marker gene products of the present invention. The method of screening comprises obtaining samples from subjects diagnosed with or suspected of having asthma, contacting each separate aliquot of the samples with one or more of a plurality of test compounds, and comparing expression of one or more marker gene products in each of the aliquots to determine whether any of the test compounds provides a substantially decreased level of expression or activity of a marker gene product relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating with the binding of a marker gene product and a binding partner, by combining the test compound, the marker gene product, and binding partner together and determining whether binding of the binding partner and the marker gene product occurs. The test compound may be either a small molecule or a biomolecule.

Modulators of marker gene product expression, activity or binding ability are useful as therapeutic compositions of the invention. Such modulators (e.g., antagonists or agonists) may be formulated as pharmaceutical compositions, as described herein below. Such modulators may also be used in the methods of the invention, for example, to diagnose, treat, or prognose asthma.

The invention provides methods of conducting high-throughput screening for test compounds capable of inhibiting activity or expression of a marker gene product of the present invention. In one embodiment, the method of high-throughput screening involves combining test compounds and the marker gene product and detecting the effect of the test compound on the marker gene product.

A variety of high-throughput functional assays well-known in the art may be used in combination to screen and/or study the reactivity of different types of activating test compounds. Since the coupling system is often difficult to predict, a number of assays may need to be configured to detect a wide range of coupling mechanisms. A variety of fluorescence-based techniques is well-known in the art and is capable of high-throughput and ultra high throughput screening for activity, including but not limited to BRET™ or FRET™ (both by Packard Instrument Co., Meriden, Conn.). The ability to screen a large volume and a variety of test compounds with great sensitivity permits for analysis of the therapeutic targets of the invention to further provide potential inhibitors of asthma. The BIACORE™ system may also be manipulated to detect binding of test compounds with individual components of the therapeutic target, to detect binding to either the encoded protein or to the ligand.

Therefore, the invention provides for high-throughput screening of test compounds for the ability to inhibit activity of a protein encoded by the marker gene products listed in Tables 6, 7a, 7b, 8a, or 8b, by combining the test compounds and the protein in high-throughput assays such as BIACORE™, or in fluorescence-based assays such as BRET™. In addition, high-throughput assays may be utilized to identify specific factors which bind to the encoded proteins, or alternatively, to identify test compounds which prevent binding of the receptor to the binding partner. In the case of orphan receptors, the binding partner may be the natural ligand for the receptor. Moreover, the high-throughput screening assays may be modified to determine whether test compounds can bind to either the encoded protein or to the binding partner (e.g., substrate or ligand) which binds to the protein.

In one embodiment, the high-throughput screening assay detects the ability of a plurality of test compounds to bind to a marker gene product selected from the group consisting of the markers listed in Tables 6, 7a, 7b, 8a, or 8b. In another specific embodiment, the high-throughput screening assay detects the ability of a plurality of a test compound to inhibit a binding partner (such as a ligand) to bind to a marker gene product selected from the group consisting of the markers listed in Tables 6, 7a, 7b, 8a, or 8b. In yet another specific embodiment, the high-throughput screening assay detects the ability of a plurality of a test compounds to modulate signaling through a marker gene product selected from the group consisting of the markers listed in Tables 6, 7a, 7b, 8a, or 8b. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051.

In one embodiment, one or more candidate agents are administered in vitro directly to cells derived from healthy volunteers and/or asthma patients (either before or after treatment). In another particular embodiment, healthy volunteers and/or asthma patients are administered one or more candidate agent directly in any manner currently known to, and commonly used by the skilled artisan including generally, but not limited to, enteral or parenteral administration.

### Electronic Systems

The present invention also features electronic systems useful for the prognosis, diagnosis, or selection of treatment of asthma. These systems include an input or communication device for receiving the expression profile of a patient of interest or the reference expression profile(s). The reference expression profile(s) can be stored in a database or other media. The comparison between expression profiles can be conducted electronically, such as through a processor or computer. The processor or computer can execute one or more programs which compare the expression profile of the patient of interest to the reference expression profile(s), the programs can be stored in a memory or other storage media or downloaded from another source, such as an internet server. In one example, the electronic system is coupled to a nucleic acid array and can receive or process expression data generated by the nucleic acid array. In another example, the electronic system is coupled to a protein array and can receive or process expression data generated by the protein array.

### Kits for Prognosis, Diagnosis, or Selection of Treatment of Asthma

In addition, the present invention features kits useful for the diagnosis or selection of treatment of asthma. Each kit includes or consists essentially of at least one probe for an asthma marker (e.g., a marker selected from Tables 6, 7a, 7b, 8a, or 8b). Reagents or buffers that facilitate the use of the kit can also be included. Any type of probe can be used in the present invention, such as hybridization probes, amplification primers, antibodies, or any and all other probes commonly used and known to the skilled artisan. In one embodiment, the asthma markers are selected from Table 7b. In some embodiments, the asthma markers are selected from Table 6. In one embodiment of the present invention, the asthma markers are selected from the markers indicated in Table 7b with an FDR for association with asthma in PBMCs prior to culture of less than 0.051.

In one embodiment, a kit of the present invention includes or consists essentially of at least 1, 2, 3, 4, 5, 6, 7, 8, 9, 10, or more polynucleotide probes or primers. Each probe/primer can hybridize under stringent conditions or nucleic acid array hybridization conditions to a different respective asthma marker. As used herein, a polynucleotide can hybridize to a gene if the polynucleotide can hybridize to an RNA transcript, or complement thereof, of the gene. In another embodiment, a kit of the present invention includes one or more antibodies, each of which is capable of binding to a polypeptide encoded by a different respective asthma prognostic or disease gene/marker.

In one example, a kit of the present invention includes or consists essentially of probes (e.g., hybridization or PCR amplification probes or antibodies) for at least 1, 2, 3, 4, 5, 10, 14, 20, 25, 30, 35, 40, 45, 50, 55, 60, 65, 70, 75, or more genes selected from Tables 6, 7a, 7b, 8a, or 8b. In another embodiment, the kit can contain nucleic acid probes and antibodies to 1, 2, 3, 4, 5, 10, 14, 20, 25, 30, 35, 40, 45, 50, 55, 60, 65, 70, 75, or more genes selected from Tables 6, 7a, 7b, 8a, or 8b.

The probes employed in the present invention can be either labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The kits of the present invention can also have containers containing buffer(s) or reporter means. In addition, the kits can include reagents for conducting positive or negative controls. In one embodiment, the probes employed in the present invention are stably attached to one or more substrate supports. Nucleic acid hybridization or immunoassays can be directly carried out on the substrate support(s). Suitable substrate supports for this purpose include, but are not limited to, glasses, silica, ceramics, nylons, quartz wafers, gels, metals, papers, beads, tubes, fibers, films, membranes, column matrices, or microtiter plate wells. The kits of the present invention may also contain one or more controls, each representing a reference expression level of a marker detectable by one or more probes contained in the kits.

The present invention also allows for personalized treatment of asthma. Numerous treatment options or regimes can be analyzed according to the present invention to identify markers for each treatment regime. The peripheral blood expression profiles of these markers in a patient of interest are indicative of the clinical outcome of the patient and, therefore, can be used for the selection of treatments that have favorable prognoses of the majority of all other available treatments for the patient of interest. The treatment regime with the best prognosis can also be identified.

Treatment selection can be conducted manually or electronically. Reference expression profiles or gene classifiers can be stored in a database. Programs capable of performing algorithms such as the k-nearest-neighbors or weighted voting algorithms can be used to compare the peripheral blood expression profile of a patient of interest to the database to determine which treatment should be used for the patient.

It should be understood that the above-described embodiments and the following examples are given by way of illustration, not limitation. Various changes and modifications within the scope of the present invention will become apparent to those skilled in the art from the present description.

### EXAMPLE 1

### Clinical Trial and Data Collection

### Demographics of Subjects

Twenty-six (26) subjects with asthma and eleven (11) healthy volunteer subjects were recruited for this study. Asthma subjects were from the Allergy, Asthma and Dermatology Research Center in Lake Oswego, Oreg. and Bensch Research Associates in Stockton, Calif. Healthy volunteers were from Wyeth Research in Cambridge, Mass. Each clinical site's institutional review board or ethics committee approved this study, and no study-specific procedures were performed before obtaining informed consent from each subject. All asthma subjects were on standard of care treatment of inhaled steroids, and samples collected included 4 (15%) from patients on systemic steroids. Asthma subjects were categorized as mild persistent, moderate persistent or severe persistent according to the 1997 NIH Guidelines for the Diagnosis and Management of Asthma. In all, 19 of the asthma subjects were allergic, with the remainder non-allergic. Atopic status in 20 of 26 asthma subjects was assessed by clinical investigators based on positive skin test, family history or clinical assessment. Healthy volunteers had no known history of asthma or seasonal allergies. Demographic information for the subjects is shown in Table 4.

### Sample Collection

PBMCs from asthma subjects at selected clinical sites participating in a multi-center observational study of gene expression in asthma were isolated from whole blood samples (8 ml×6 tubes) collected into cell purification tubes (Becton Dickinson, Franklin Lakes, N.J.) according to the manufacturer's recommendations. All asthma samples where shipped at room temperature in a temperature controlled box overnight from the clinical site and processed immediately upon receipt (approximately 24 hours after blood draw). Healthy volunteer samples did not require shipping and were stored overnight before processing to mimic the conditions of the asthma samples.

### Histamine Release Assay

Leukocyte degranulation was assayed by measuring histamine release from whole blood following a 30 minute exposure to an allergen cocktail. As a positive control, histamine release in the presence of IgE cross-linked with anti-human IgE (KPL, Gaithersburg, Md.) was measured. Ninety-four percent of subjects in this study demonstrated positive responses in the control histamine release assay with cross-linked IgE. Histamine was measured by ELISA (Beckman Coulter, Fullerton, Calif.) and results reported as a percent of total histamine release, determined triton-X lysis of whole blood.

### In Vitro Cell Stimulation

PBMCs were stimulated in vitro with a cocktail containing 4 different allergens from house dust mite, ragweed and cat. Recombinant allergens, Der p1, Der f2, Fel d1 (Indoor Biotech, Charlottesville, Va.) and natural ragweed allergen (Allergy Lab, Seattle, Wash.) were selected and screened for endotoxin contamination (LAL Endotoxin Test, Catalog #HIT302, sensitivity, 0.0001 Eu/ml, Cell Sciences, Canton, Mass.). The sensitivity of the subjects was unknown but the allergens were chosen based on the estimate that 80% of allergic individuals are believed to react to one or more of these allergens. Culture medium contained RPMI-1640 (Sigma) with 10% heat inactivated FCS (Sigma St. Louis, Mo.) and 100 unit/mL Penicillin and 100 mg/mL Streptomycin and 0.292 mg/mL Glutamine (GIBCO RL Invitrogen, Carlsbad, Calif.). The final allergen cocktail concentrations in culture medium were: Der p1 and Der f2 (dust mite), 1 mg/ml; Fel d1 (cat), 1.25 mg/ml; ragweed, 125 mg/ml. The total level of endotoxin contamination in culture medium was 0.057 Eu/ml. The cPLA2 inhibitor 4-{3-[1-benzhydryl-5-chloro-2-(2-{[(2,6-dimethylbenzyl)sulfonyl]amino}ethyl)-1H-indol-3-yl]propyl}benzoic acid was used at a concentration of 0.3 μM/ml. Zileuton, a 5-lipoxygenase inhibitor, was added at a concentration of 5 μM. The inhibitory activity of both the cPLA2 inhibitor and Zileuton samples were verified in a human whole blood assay. After 6 days in culture approximately 200 μL of supernatant was removed using an 8-channel pipettor without disturbing the cell pellet and placed into a collection plate for cytokine ELISA assays. To the remaining cell pellet 100 μL of RLT lysis buffer containing 1% beta-mercaptoethanol was added and snap frozen for RNA purification.

### Cytokine Assays

Levels of γIFN, IL-5 and IL-13 in supernatants were measured by ELISA following 6 days in culture. Allergen-specific levels were determined by comparing levels in the presence and absence of allergen. Supernatant was added to pre-coated γIFN, IL5 and IL13 ELISA plates (Pierce Endogen, Meridain Rockford, Ill.) according to the manufacturer's instructions. The appropriate biotinylated antibody for each cytokine was used and streptavidin-HRP was added and developed using TMB substrate solution. Absorbance was measured by subtracting the 550 nm values from 450 nm values. Results were calculated using Softmax 4.7 software. The sensitivity of the assays was also within the limits of the manufacturer guidelines. The limit of detection was 2 pg/ml for IL-5, 7 pg/ml for IL-13, and 2 pg/ml for γIFN.

### RNA Purification and Microarray Hybridization

RNA was purified using QIA shredders and Rneasy mini kits (Qiagen, Valencia, Calif.). PBMC pellets frozen in RLT lysis buffer containing 1% β-mercaptoethanol were thawed and processed for total RNA isolation using the QIA shredder and RNeasy mini kit. A phenol:chloroform extraction was then performed, and the RNA was repurified using the RNeasy mini kit reagents. Eluted RNA was quantified using a Spectramax96 well plate UV reader (Molecular Devices, Sunnyvale, Calif., USA) monitoring A260/280 OD values. The quality of each RNA sample was assessed by capillary electrophoresis alongside an RNA molecular weight ladder on the Agilent 2100 bioanalyzer (Agilent Technologies, Palo Alto, Calif., USA). RNA samples were assigned quality values of intact (distinct 18S and 28S bands); partially degraded (discernible 18S and 28S bands with presence of low molecular weight bands) or completely degraded (no discernible 18S and 28S bands).

Labeled targets for oligonucleotide arrays were prepared using a modification of the procedure described by Lockhart et al. (Lockhart (1996) Nat. Biotechnol. 14:1675-80). Labeled targets were hybridized to the HG-U133A Affymetrix GeneChip Array as described in the Affymetrix technical manual. Eleven biotinylated control transcripts ranging in abundance from 3 parts per million (ppm) to 100 ppm were spiked into each sample to function as a standard curve (Hill (2001) Genome Biol. 2:RESEARCH0055). GeneChip MAS 5.0 software was used to evaluate the hybridization intensity, compute the signal value for each probe set and make an absent/present call.

### Data Normalization and Filtering

GeneChips were required to pass the pre-set quality control criteria that the RNA quality metric required a 5′:3′ ratio. Two asthma subjects were excluded from the study due to failure to meet the RNA quality metric and 2 GeneChips from the group treated with cPLA2a inhibitor were excluded for the same reason. The signal value for each probe set was converted into a frequency value representative of the number of transcripts present in 10⁶ transcripts by reference to the standard curve (Hill (2001) Genome Biol. 2:RESEARCH0055). Data for 10280 probe sets that were called “present” in at least 5 of the samples and with a frequency of 10 ppm or more in at least 1 of the samples were subject to the statistical analysis described below, while probe sets that did not meet this criteria were excluded.

### Statistical Analysis

The antigen dependent fold change differences were calculated by determining the difference in the log 2 frequency in the presence and absence of antigen. ANOVA was performed using this metric to identify allergen dependent differences, and also to identify significant differences between the asthma and healthy volunteer groups with respect to the response to allergen. Raw P-values were adjusted for multiplicity according to the false discovery rate (FDR) procedure of Benjamini and Hochberg (Reiner (2003) Bioinformatics 19:368-75) using Spotfire (Somerville, Mass.). Significant effects of the cPLA2 inhibitor 4-{3-[1-benzhydryl-5-chloro-2-(2-{[(2,6-dimethylbenzyl)sulfonyl]amino}ethyl)-1H-indol-3-yl]propyl}benzoic acid were identified by ANOVA comparing the log 2 differences in the groups treated with allergen to the groups treated with allergen and the cPLA2 inhibitor.

### Hierarchical Clustering

For hierarchical agglomerative clustering of probesets and arrays, the Log-2 scale MAS5 expression values from each probeset were first z-normalized so that each probeset had a mean expression level of zero and a standard deviation of one across all samples. Then these normalized profiles were clustered hierarchically using UPGMA (unweighted average link) and the Euclidean distance measure.

### Ingenuity Pathways Analysis

Data were analyzed through the use of Ingenuity Pathways Analysis (IPA) (Ingenuity® Systems, www.ingenuity.com) Asthma-associated gene identifiers and corresponding expression and p values were uploaded into in the application. Gene identifiers were mapped to the corresponding gene objects in the Ingenuity Pathways Knowledge Base. The Focus genes were overlaid onto a global molecular network developed from information contained in the Ingenuity Pathways Knowledge Base. Networks of these Focus Genes were then algorithmically generated based on their connectivity. Functional analysis, Canonical pathways as well as annotations for these genes were also obtained using IPA.

### EXAMPLE 2

### Determination of Disease-Related Transcripts in Volunteers

### In Vitro Histamine Release Occurs in Both Populations

An important aspect of the inflammatory response is the release of granules by leukocytes. In particular, histamine is released by basophils and mast cells in response to allergen. Whole blood samples obtained from healthy and asthmatic volunteers were treated with allergen for thirty minutes and histamine release was measured. Allergen induced histamine release was compared to histamine release in response to anti-human IgE. The antibody causes non-specific degranulation through the cross-linking of IgE present on the surface. Samples that had a positive response to IgE cross-linking were subsequently tested in a histamine release assay in response to allergen. In the healthy population, eight of the eleven tested positive in the control experiment and only one was responsive to allergen. In the asthmatic population, fifteen of twenty-six were positive in the control assay. Eleven samples were tested in response to allergen and only five responded specifically to allergen.

### In Vitro Cytokine Production in Response to Allergen

We determined the allergen responsiveness of the peripheral blood mononuclear cells (PBMC) by measuring the levels of cytokines produced by the PBMC of asthma and healthy subjects following 6 days of in vitro stimulation. ELISA analyses were carried out for IFN-gamma, IL-5, and IL-13. All healthy volunteers showed a cytokine response to allergen defined as a two-fold or greater increase in the production of at least one cytokine compared to baseline levels. In the asthma group, approximately eighty percent had a cytokine response to allergen (Table 5). Table 5 shows the range of response for the two populations. According to Table 5, production of cytokine was measured using ELISA assays on the supernatant from PBMC cultures after 6-day allergen stimulation as described. Subjects were classified as positive responders if cytokine production was increased at least 2 fold over baseline in the presence of allergen and/or had a positive score in the histamine release assay. There was no statistical difference (P value <0.05) found between asthma and healthy groups with respect to allergen-induced production of these cytokines.

### PBMC Expression Profile/Allergen Response Study: Asthmatics and Healthy Volunteers

Transcriptional profiling was done on RNA collected from allergen-treated PBMCs from the asthmatic and healthy volunteers and gene expression levels were measured as described above. There were 10280 probesets that were called present in at least 5 samples and a frequency greater than 10 ppm and these were selected for further analysis. From these we identified the genes that showed a similar response to allergen in both the asthmatic and healthy groups. Genes in this category had an allergen dependent fold change ≧1.5, and had no significant difference FDR≧0.051 between the two groups with respect to allergen-dependent changes. There were 133 probesets (representing 123 unique genes) that met these criteria. The complete list of probes and their descriptions are included in Table 7a. The fourth column of Table 7a indicates the FDR for the significance of the association of genes with asthma in PBMCs prior to culture (that is, untreated PBMCs) when profiles were compared between asthmatics and healthy volunteers. Genes that were up regulated in both populations included those involved in the immune response and cell growth. For example, interleukin-9 (IL9) (Godfraind (1998) J. Immunol. 160:3989-96; Louahed (2001) Blood 97:1035-42; Temann (1998) J. Exp. Med. 188:1307-20; Vink (1999) J. Exp. Med. 189:1413-23) and chemokine (C-X-C motif) ligand 3 (CXCL3) (Geiser (1993) J. Biol. Chem. 268:15419-24; Inngjerdingen (2001) Blood 97:367-75) are immune system genes that are involved in chemotaxis and activation of lymphoid cells that are up-regulated in both populations but were up-regulated to a greater extent in the asthma subjects. Genes down-regulated in response to allergen included those implicated in degradation of the extracellular matrix, matrix metalloproteases-2 and 12 (MMP2, MMP12) (Sternlicht (2001)Annu. Rev. Cell Dev. Biol. 17:463-516).

Comparison of the expression levels of the 10280 probesets in the asthma and healthy subjects identified 167 probesets (representing 153 unique genes) whose allergen-dependent changes differed significantly (FDR<0.051) between asthma and healthy subjects. These genes also showed an allergen-dependent fold change >1.5 in at least one group. The complete list of the 167 probe sets and, for each, the significance of the difference between the groups is shown in Table 7b. The fourth column of Table 7b indicates the FDR for the significance of the association of genes with asthma in PBMCs prior to culture (that is, untreated PBMCs) when profiles were compared between asthmatics and healthy volunteers. A visualization of the differences between asthma and healthy subjects with respect to allergen-dependent changes in expression level of all 167 probesets is shown in FIG. 1. The visualization was generated using an algorithm that groups subjects based on the similarities with respect to allergen dependent gene expression changes. With one exception, all the healthy subjects were grouped together, and 22 of the 26 asthma subjects were grouped together. Table 6 shows 50 genes—a subset of genes that showed a significant difference between asthma and healthy subjects with respect to the response to allergen. The genes shown in Table 6 were associated with an allergen response of 1.5 fold or more in the asthma group, while having a less than 1.1 fold response to allergen in the healthy volunteer population. In this list are genes previously associated with the asthmatic phenotype including the Zap70 and LCK tyrosine kinases (Wong (2005) Curr. Opin. Pharmacol. 5:264-71), the toll like receptor 4 (TLR4) (Hollingsworth (2004) Am. J. Respir. Crit. Care Med. 170:126-32; Rodriguez (2003) J. Immunol. 171:1001-8) and complement component 3a receptor 1 (C3AR1) (Bautsch (2000) J. Immunol. 165:5401-5; Drouin (2002) J. Immunol. 169:5926-33; Hasegawa (2004) Hum. Genet. 115:295-301; Humbles (2000) Nature 406:998-1001; Zimmermann (2003) J. Clin. Invest. 111:1863-74). Allergen-responsive genes not previously shown to be involved in the asthma phenotype included sialoadhesin (SN1-CD163) (Fabriek (2005) Immunobiology 210:153-60), interleukin-21 receptor (IL21R) (Mehta (2004) Immunol. Rev. 202:84-95), and a disintegrin/metalloprotease, ADAM19 (Fritsche (2000) Blood 96:732-9).

### EXAMPLE 3

### Transcriptional Effects of Therapy

cPLA2 Inhibitor Therapy Alters the Expression Profiles in Response to Allergen

The transcriptional effect of cPLA2 inhibition on expression of the 167 allergen-asthma specific probesets was determined. The asthma specific gene expression was altered in the presence of the inhibitor 4-{3-[1-benzhydryl-5-chloro-2-(2-{[(2,6-dimethylbenzyl)sulfonyl]amino}ethyl)-1H-indol-3-yl]propyl}benzoic acid (hereinafter “the cPLA2 inhibitor”) when compared to the allergen treatment alone. The complete analysis results, including fold changes, with and without cPLA2 inhibition is listed in Tables 7a and 7b. With the exception of a few probes, the probe set falls into two distinct categories. In the first category, probes that correspond to genes that were up-regulated in asthma samples in response to allergen, such as ZAP70, LCK, and MCM 2, are reduced to the levels seen in the allergen treated healthy controls. In the second category, genes that were initially down regulated in the asthma samples in the presence of allergen, such as sialoadhesin (SN), CD84, and tissue inhibitor of metalloproteinase 3 (TIMP3) are up-regulated in the presence of inhibition. A hierarchical cluster analysis was performed to visualize the differences associated with cPLA2a inhibition for the 167 asthma-associated probe sets (FIG. 2). The analysis identified three separate groups based on similarities in gene expression pattern: 1) asthma samples treated with allergen, 2) asthma samples treated with allergen and the cPLA2 inhibitor and 3) a small population of samples allergen-treated and allergen+the cPLA2 inhibitor treated. Interestingly, group 3 contains the same subjects who originally clustered with the healthy samples in response to allergen (see FIG. 1).

cPLA2 Inhibition has a Minimal Effect on Base Line Expression of Genes in Asthmatics

cPLA2 inhibition does not affect gene expression in the absence of allergen stimulation in the asthmatic population. Only three genes met the filtering cut off of an FDR less than equal to 0.051 and 1.5 or greater fold change (Table 8a), representing an unknown gene, a pituitary specific gene, PACAP, and a hormone, PMCH. In the healthy population, 36 probes were significantly upregulated in the presence of cPLA2 inhibition and 43 probes were significantly upregulated in the presence of cPLA2 and 43 probes were significantly downregulated in the presence of cPLA2 inhibition (Table 8b).

### Functional Annotation of Gene Expression

To explore the functional relatedness of the allergen responsive genes and identify associated pathways, the asthma specific-allergen gene list, (167 probeset) was functionally annotated by Ingenuity Pathways Analysis (IPA). Of the 167 probes initially entered into the analysis, 127 met the criteria for pathway analysis. The criteria are based on the Ingenuity knowledge base and on our previous statistical analysis. Seven well-populated functional networks were created based on this information. The top functions for the networks created using IPA include immune and lymphatic system development and function, immune response, DNA replication, recombination and repair. The top-scoring network (Network 1) consisted of 35 nodes that represent genes involved in immune response and cell cycle (FIG. 3(a)). Genes in this network involved in the immune response were up regulated in the asthmatics compared to the healthy subjects including the T cell receptor signaling genes CD3D, CD28, and ZAP70 (Kuhns (2006) Immunity 24:133-9); Wang (2004) Cell Mol. Immunol. 1:37-42; Zamoyska (2003) Immunol. Rev. 191:107-18). As expected, the expression levels (node color intensities) in Network 1 for the healthy volunteer population looked very different from the asthma subjects. However, in the healthy subjects, a few of the genes were down regulated similarly to the asthma subjects, but to a significantly lesser extent. This set of genes includes cathepsin B (CTSB), tissue inhibitor of metalloproteinase 3 (TIMP3) and CD36 antigen (collagen type I receptor, thrombospondin receptor) (CD36) (FIG. 3(b)).

The striking effect of cPLA2 inhibition on allergen-induced gene expression changes in the asthma group can be illustrated by utilizing Ingenuity Pathways Analysis. In this analysis, the expression values obtained in the presence of the inhibitor were overlaid into the gene set created based on asthma specific allergen gene changes. Every single probe in Network 1 in the asthmatic population has an altered level of expression in the presence of the inhibitor (FIG. 3(c)). In the healthy population, the few genes that were down regulated in response to allergen in Network 1 are brought up to non-allergen-stimulated background levels in the presence of the inhibitor (data not shown).

### EXAMPLE 4

### Clinical Application of Expression Profiling

Patients manifesting the potential symptoms of asthma are observed by a physician and blood is drawn for diagnosis and a determination of asthma severity, if any. PBMCs are isolated from whole blood samples (8 ml×6 tubes) and are collected into cell purification tubes (Becton Dickinson, Franklin Lakes, N.J.) according to the manufacturer's recommendations. trampline

Optionally, PBMCs are stimulated in vitro with a cocktail containing 4 different allergens from house dust mite, ragweed, and cat. Recombinant allergens, Der p1, Der f2, Fel d1 (Indoor Biotech, Charlottesville, Va.) and natural ragweed allergen (Allergy Lab, Seattle, Wash.) are selected and screened for endotoxin contamination (LAL Endotoxin Test, Catalog #HIT302, sensitivity, 0.0001 Eu/ml, Cell Sciences, Canton, Mass.). The allergens are chosen based on the estimate that 80% of allergic individuals are believed to react to one or more of these allergens. The culture medium contains RMPI-1640 (Sigma) with 10% heat inactivated fetal calf serum (FCS) (Sigma, St. Louis, Mo.) and 100 unit/mL penicillin and 100 mg/mL streptomycin and 0.292 mg/mL glutamine (GIBCO RL Invitrogen, Carlsbad, Calif.). The final allergen cocktail concentrations in culture medium are: Der p1 and Der f2 (dust mite), 1 mg/ml; Fel d1 (cat), 1.25 mg/ml; ragweed, 125 mg/ml. Optionally, the physician or clinical associates working under her direction may add a cPLA2 inhibitor, such as 4-{3-[1-benzhydryl-5-chloro-2-(2-{[(2,6-dimethylbenzyl)sulfonyl]amino}ethyl)-1H-indol-3-yl]propyl}benzoic acid, to the medium at a concentration of approximately 0.3 μM/ml. Optionally, the physician or clinical associates working under her direction may further add Zileuton to the medium at a concentration of approximately 5 μM.

RNA is purified from inhibitor/allergen-treated or untreated PBMCs using QIA shredders and RNeasy mini kits (Qiagen, Valencia, Calif.). PBMC pellets frozen in RLT lysis buffer containing 1% β-mercaptoethanol are thawed and processed for total RNA isolation using the QIA shredder and Rneasy mini kit. A phenol:chloroform extraction is then performed, and the RNA is repurified using the Rneasy mini kit reagents. Eluted RNA is quantified using a Spectramax96 well plate UV reader (Molecular Devices, Sunnyvale, Calif., USA) monitoring the A260/280 OD values. The quality of each RNA sample is assessed by capillary electrophoresis alongside an RNA molecular weight ladder on the Agilent 2100 bioanalyzer (Agilent Technologies, Palo Alto, Calif., USA). RNA samples are assigned quality values of intact (18S and 28S bands); partially degraded (discernible 18S and 28S bands with presence of low molecular weight bands) or completely degraded (no discernible 18S and 28S bands).

Labeled targets for oligonucleotide arrays are prepared using a modification of the procedure described by Lockhart et al. (Lockhart (1996) Nat. Biotechnol. 14:1675-80). Labeled targets are hybridized to an array using standard methods known in the art, the array including probes for the markers ZWINT, FLJ23311, PRC1, RANBP5, CD3D, MELK, RACGAP1, PSIP1, TACC3, BCCIP, OIP5, PRKDC, HNRPUL1, IL-21R, RAD21 homologue, PTTG1, C6ORF149, SNRPD3, FYN, GM2A, SLC36A1, TM6SF1, PYGL, PLEKHB2, CD84, GCHFR, SORT1, SLCO2B1, ZFYVE26, RNF13, PRNP, GAS7, ATP6V1A, and ATP6V0D1. Eleven biotinylated control transcripts ranging in abundance from 3 parts per million (ppm) to 100 ppm are spiked into each sample to function as a standard curve (Hill (2001) Genome Biol. 2:RESEARCH0055). The signal value for each probe is converted into a frequency value representative of the number of transcripts present in 10⁶ transcripts by reference to the standard curve. (Hill (2001) Genome Biol. 2:RESEARCH0055) Software commonly employed in the art for pharmacogenomic analysis is used to evaluate the hybridization intensity, compute the signal value for each probe set, and make an absent/present call. Arrays are required to pass the pre-set quality control criteria that the RNA quality metrics required a 5′:3′ ratio.

The allergen-dependent fold change differences in marker expression levels are calculated by determining the difference in the log 2 frequency in the presence and absence of allergen. The physician may also provide a diagnosis or severity assessment by comparing the expression level of the marker or markers observed as compared to reference expression levels of the marker or markers. The reference expression levels are preferably known basal expression levels of the marker or markers derived from healthy volunteers in clinical studies. The physician can make a diagnosis by determining the extent to which a given marker is upregulated or downregulated compared to a reference level. The physician can assess the severity of the condition, if any, by comparing the expression levels of particular markers linked to severity to a reference expression level.

In lieu of in vitro inhibitor administration and in vitro allergen challenge, the physician may provide the patient with an agent, such as an inhibitor. Patients with moderate to severe cases of asthma are treated with a cPLA2 inhibitor, such as 4-{3-[1-benzhydryl-5-chloro-2-(2-{[(2,6-dimethyl benzyl)sulfonyl]amino}ethyl)-1H-indol-3-yl]propyl}benzoic acid, at a concentration of approximately 0.3 μM/ml as a once daily dose. At her election, the physician may also administer Zileuton at a concentration of approximately 5 μM as a once daily dose. Clinical staging and severity of the disease are recorded prior to every treatment and every 2-3 weeks following initiation of cPLA2 inhibitor therapy. Blood is drawn and PBMCs isolated at every patient visit prior to cPLA2 inhibitor (and optionally Zileuton) administration. Expression levels of the marker or markers of interest are then determined as described above. The effectiveness of the treatment is therefore assessed after every patient visit and a determination is made as to continuation of the treatment or alteration of the treatment regimen.

The following tables, which are referenced in the foregoing description, are herein incorporated in their entirety.

TABLE 1
ALLERGY DRUGS IN DEVELOPMENT OR ON THE MARKET

| MARKETER | BRAND NAME (Generic Name) | MECHANISM |
|---|---|---|
| Schering- | Claritin & Claritin D (loratidine) | Anti-histamine |
| Plough | | |
| UCB | Vancenase (beclomethasone) | Steroid |
| | Reactine (cetirizine) (US) | Anti-histamine |
| | Zyrtec (cetirizine) (ex US) | |
| | Longifene (buclizine) | Anti-histamine |
| | UCB 28754 (ceterizine alalogue) | Anti-histamine |
| Glaxo | Beconase (beclomethasone) | Steroid |
| | Flonase (fluticasone) | Steroid |
| Aventis | Allegra (fexofenadine) | Anti-histamine |
| | Seldane (terfenadine) | |
| Pfizer | Reactine (cetirizine) (US) | Anti-histamine |
| | Zyrtec/Reactine (cetirizine) | |
| | (ex US) | |
| Sepracor | Allegra (fexofenadine) | Anti-histamine |
| | Desloratadine | Anti-histamine |
| | Cetirizine (—) | Anti-histamine |
| | Norastemizole | |
| B. Ingelheim | Alesion (epinastine) | Anti-histamine |
| Aventis | Kestin (ebastine) (US) | |
| | Bastel (ebastine) (Eu/Ger) | |
| | Nasacort (tramcinolone) | Steroid |
| Johnson & | Hismanol (estemizole) | Anti-histamine |
| Johnson | | |
| | Livostin/Livocarb (levocabastine) | Anti-histamine |
| AstraZeneca | Rhinocort (budesonide) (Astra) | Steroid |
| Merck | Rhmocort (budesonide) | Steroid |
| Eisai | Azeptin (azelastine) | Anti-histamine |
| Kissei | Rizaben (tranilast) | Anti-histamine |
| Shionogi | Triludan (terfenadine) | Anti-histamine |
| | S-5751 | |
| Schwarz | Zolim (mizolastine) | Anti-histamine |
| Daiichi | Zyrtec (cetirizine) (ex US) | Anti-histamine |
| Tanabe | Talion/TAU-284 (betatastine) | Anti-histamine |
| Sankyo | CS 560 (Hypersensitizaion therapy | Other |
| | for cedar pollen allergy) | |
| Asta Medica | Azelastine-MDPI (azelastine) | Anti-histamine |
| BASF | HSR 609 | Anti-histamine |
| SR Pharma | SRL 172 | Immunomodulation |
| Peptide | Allergy vaccine (allergy (hayfever, | Downregulates IgE |
| Therapeutics | anaphylaxis, atopic asthma)) | |
| Peptide | Tolerizing peptide vaccine (rye | Immuno-suppressant |
| Therapeutics | grass peptide (T cell epitope)) | |
| Coley | CpG DNA | Immunomodulation |
| Pharmaceutical | | |
| Group | | |
| Genetech | Anti-IgE | Down-regulator |
| | | of IgE |
| SR Pharma | SRL 172 | Immunomodulation |

TABLE 2
ASTHMA DRUGS IN DEVELOPMENT OR ON THE MARKET

| | BRAND NAME (Generic | |
|---|---|---|
| MARKETER | Name) | MECHANISM |
| Glaxo | Serevent (salmeterol) | Bronchodilator/beta-2 agonist |
| | Flovent (fluticasone) | Steroid |
| | Flixotide (fluticasone) | |
| | Becotide (betamethasone) | Steroid |
| | Ventolin (salbutamol) | Bronchodilator/beta-2 agonist |
| | Seretide (salmeterol & | Beta agonist & steroid |
| | fluticasone) | |
| | GW215864 | Steroid, hydrolysable |
| | GW250495 | Steroid, hydrolysable |
| | GW28267 | Adenosine A2a receptor agonist |
| AstraZeneca | Bambec (bambuterol) (Astra) | |
| | Pulmicort (budesonide) (Astra) | Steroid |
| | Bricanyl Turbuhaler | Bronchodilator/beta-2 agonist |
| | (terbutaline) (Astra) | |
| | Accolate (zafurlukast) (Zeneca) | Leukotriene antagonist Clo-Phyllin |
| | | (theophylline) |
| | Inspiryl (salbutamol) (Astra) | Bronchodilator/beta-2 agonist |
| | Oxis Turbuhaler | Bronchodilator/beta-2 agonist |
| | (D2522/formoterol) | |
| | Symbicort (pulmicort-oxis | Steroid |
| | combination) | |
| | Roflepanide (Astra) | Steroid |
| | Bronica (seratrodast) | Thromboxane A2 synthesis inhibitor |
| | ZD 4407 (Zeneca) | 5 lipoxygenase inhibitor |
| B. Ingelheim | Atrovent (Ipratropium) | Bronchodilator/anti-cholinergic |
| | Berodual (ipratropium & | Bronchodilator/beta-2 agonist |
| | fenoterol) | |
| | Berotec (fenoterol) | Bronchodilator/beta-2 agonist |
| | Alupent (orciprenaline) | Bronchodilator/beta-2 agonist |
| | Ventilat (oxitropium) | Bronchodilator/anti-cholinergic |
| | Spiropent (clenbuterol) | Bronchodilator/beta-2 agonist |
| | Inhacort (flunisolide) | Steroid |
| | B1679/tiotropium bromide | |
| | RPR 106541 | Steroid |
| | BLIX 1 | Potassium channel |
| | BIIL284 | LTB-4 antagonist |
| Schering- | Proventil (salbutamol) | Bronchodilator/beta-2 agonist |
| Plough | | |
| | Vanceril (becbomethasone) | Steroid |
| | Mometasone furoate | Steroid |
| | Theo-Dur (theophylline) | |
| | Uni-Dur (theophylline) | |
| | Asmanex (mometasone) | Steroid |
| | CDP 835 | Anti-IL-5 Mab |
| RPR | Intal (disodium cromoglycate) | Anti-inflammatory |
| (Aventis) | Inal/Aarane (disodium | |
| | cromoglycate) | |
| | Tilade (nedocromil sodium) | |
| | Azmacort (triamcinolone | Steroid |
| | acetonide) | |
| | RP 73401 | PDE-4 inhibitor |
| Novartis | Zaditen (ketotifen) | Anti-inflammatory |
| | Azmacort (triamoinolone) | Steroid |
| | Foradil (formoterol) | Bronchodilator/beta-2 agonist |
| | E25 | Anti-IgE |
| | KCO 912 | K+ Channel opener |
| Merck | Singulair (montelukast) | Leukotriene antagonist Clo-Phyllin |
| | | (theophylline) |
| | Pulinicort Turbuhaler | Steroid |
| | (budesonide) | |
| | Slo-Phyllin (theophylline) | |
| | Symbicort (Pulmicort-Oxis | Steroid |
| | combination) | |
| | Oxis Turbuhaler | Bronchodilator/beta-2 agonist |
| | (D2522/formoterol) | |
| | Roflepanide (Astra) | Steroid |
| | VLA-4 antagoinst | VLA-4 antagonist |
| ONO | Onon (pranlukast) | Leukotriene antagonist |
| | Vega (ozagrel) | Thromboxane A2 synthase inhibitor |
| Fujisawa | Intal (chromoglycate) | Anti-inflammatory |
| | FK 888 | Neurokine antagonist |
| Forest Labs | Aerobid (flunisolide) | Steroid |
| IVAX | Ventolin (salbutamol) | Bronchodilator/beta-2 agonist |
| | Becotide (beclomethasone | Steroid |
| | Easi-Breathe) | |
| | Serevent (salmeterol) | Bronchodilator/beta-2 agonist |
| | Flixotide (fluticasone) | Steroid |
| | Salbutamol Dry Powder Inhaler | Bronchodilator/beta-2 agonist |
| Alza | Volmax (salbutamol) | Bronchodilator/beta-2 agonist |
| Altana | Euphyllin (theophylline) | Xanthine |
| | Ciclesonide | Arachidonic acid antagonist |
| | BY 217 | PDE 4 inhibitor |
| | BY 9010N (ciclesonide) | Steroid (nasal) |
| Tanabe | Flucort (fluocinolone | Steroid |
| | acetonide) | |
| Seiyaku | | |
| Kissei | Domenan (ozagrel) | Thromboxane A2 synthase inhibitor |
| Abbott | Zyflo (zileuton) | |
| Asta Medica | Aerobec (beclomethasone | |
| | dipropionate) | |
| | Allergodil (azelastine) | |
| | Allergospasmin (sodium | |
| | cromoglycate reproterol) | |
| | Bronchospasmin (reproterol) | |
| | Salbulair (salbutamol sulphate) | |
| | TnNasal (triamcinolone) | Steroid |
| | Fomoterol-MDPI | Beta 2 adrenoceptor agonist |
| | Budesonide-MDPI | |
| UCB | Atenos/Respecal (tulobuerol) | Bronchodilator/beta-2 agonist |
| Recordati | Theodur (theophylline) | Xanthine |
| Medeva | Clickhalers Asmasal, Asmabec (salbutamol beclomethasone | |
| | diproprionate, dry inhaler) | |
| Eisai | E6123 | PAF receptor antagonist |
| Sankyo | Zaditen (ketofen) | Anti-inflammatory |
| | CS 615 | Leukotriene antaonist |
| Shionogi | Anboxan/S 1452 (domitroban) | Thromboxane A2 receptor antagonist |
| Yamanouchi | YM 976 | Leukotriene D4/thromboxane A2 |
| | | dual antagonist |
| 3M Pharma | Exirel (pirbuterol) | |
| Hoechst | Autoinhalers | Bronchodilator/beta-2 agonist |
| (Aventis) | | |
| SmithKline | Ariflo | PDE-4 inhibitor |
| Beecham | SB 240563 | Anti-IL5 Mab (humanized) |
| | SB 240683 | Anti-IL4 Mab |
| | IDEC 151/clenoliximab | Anti-CD4 Mab, primatised |
| Roche | Anti-IgE(GNE)/CG051901 | Down-regulator of IgE |
| Sepracor | Fomoterol (R, R) | Beta 2 adrenoceptor agonist |
| | Xopenex (levalbuterol) | Beta 2 adrenoceptor agonist |
| Bayer | BAY U 3405 (ramatroban) | Thromboxane A2 antagonist |
| | BAY 16-9996 | IL4 mutein |
| | BAY 19-8004 | PDE-4 inhibitor |
| SR Pharma | SRL 172 | Immunomodulation |
| Immunex | Nuance | Soluble IL-4 receptor |
| | | (immunomodulator) |
| Biogen | Anti-VLA-4 | Immunosuppressant |
| Vanguard | VML 530 | Inhibitor of 5-lipox activation protein |
| Recordati | Respix (zafurlukast) | Leukotriene antagonist |
| Genetech | Anti-IgE Mab | Down-regulator of IgE |
| Warner | CI-1018 | PDE 4 inhibitor |
| Lambert | | |
| Celltech | CDP 835/SCH 55700 (anti-IL- | PDE 4 inhibitor |
| | 5) | |
| Chiroscience | D4418 | PDE 4 inhibitor |
| | CDP 840 | PDE 4 inhibitor |
| AHP | Pda-641 (asthma steroid | |
| | replacement) | |
| Peptide | RAPID Technology Platform | Protease inhibitors |
| Therapeutics | | |
| Coley | CpG DNA | |
| Pharmaceutical | | |
| Group | | |

TABLE 3
STRINGENCY CONDITIONS

| | Poly- | Hybrid | Hybridization | |
|---|---|---|---|---|
| Stringency | nucleotide | Length | Temperature and | Wash Temp. |
| Condition | Hybrid | (bp)¹ | Buffer^{H} | and Buffer^{H} |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}*; 1xSSC | T_{B}*; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}*; 1xSSC | T_{D}*; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}*; 2xSSC | T_{L}*; 2xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1x SSPE is 0.15M NaCl, 10 mM NaH₂PO₄, and 1.25 mM EDTA, pH 7.4) can be substituted for SSC (1x SSC is 0.15M NaCl and 15 mM sodium citrate) in the hybridization and wash buffers.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ (° C.) =81.5 + 16.6(log₁₀[Na⁺]) + 0.41(% G + C) − (600/N), where N is the number of bases in the hybrid, and [Na⁺] is the molar concentration of sodium ions in the hybridization buffer ([Na⁺] for 1x SSC = 0.165 M).

TABLE 4
CHARACTERISTICS OF THE STUDY POPULATIONS.

| | Healthy Volunteers | Asthma Subjects |
|---|---|---|
| | (11) | (26) |
| Sex (M/F) | 7/4 | 9/17 |
| Race (Caucasian/ | 11/0 | 24/2 |
| Hispanic) | | |
| Age (y) | 28-51 | 21-73 |
| Asthma Severity | N.A. | 4 Mild |
| | | 11 Moderate |
| | | 11 Severe |

Legend:
M, Male;
F, Female;
Y, Years.
N.A. not applicable

TABLE 5
CYTOKINE PRODUCTION IN THE HEALTHY VOLUNTEER AND ASTHMATIC SUBJECTS

| | Healthy Subjects Total (11) | Range (pg/ml) | Range (pg/ml) | Asthma Subjects Total (26) | Range (pg/ml) | Range (pg/ml) |
|---|---|---|---|---|---|---|
| | (responders/total assayed) | −allergen | +allergen | (responders/total assayed) | −allergen | +allergen |
| Response to one or more | 11/11 (100%) | | | 19/23 (82.6%) | | |
| cytokine | | | | | | |
| IL-5 Responders | 4/11 (36.4%) | 6-110 | 6-148 | 11/23 (47.8%) | 6-243 | 6-174 |
| IL-13 Responders | 3/11 (27.3%) | 25-699 | 25-302 | 13 (56.5%) | 25-510 | 25-510 |
| gIFN Responders | 10/11 (90.9%) | 25-55 | 41-1080 | 16/23 (69.6%) | 25-864 | 25-836 |
| Overall Response | 11/11 (100%) | | | 21/23 (91.3%) | | |

TABLE 6A
GENE EXPRESSION DIFFERENCES BETWEEN ASTHMA AND HEALTHY SUBJECTS IN RESPONSE TO ALLERGEN

| | | | AOS FOLD | WHV FOLD |
|---|---|---|---|---|
| SYMBOL | DESCRIPTION | FUNCTION | CHANGE | CHANGE |
| ZWINT | ZW10 interactor | kinetochore function | 1.78 | 1.08 |
| FLJ23311 | FLJ23311 protein | DNA binding and inhibits cell growth | 1.77 | 1.01 |
| PRC1 | protein regulator of cytokinesis 1 | cytokinesis | 1.74 | 1.09 |
| CD28 | CD28 antigen (Tp44) | Antigen processing | 1.74 | 1.09 |
| PCNA | proliferating cell nuclear antigen | DNA synthesis | 1.73 | 1.03 |
| RANBP5 | karyopherin (importin) beta 3 | Nucleocytoplasmic transport | 1.72 | 1.06 |
| ZAP70 | zeta-chain (TCR) associated protein kinase 70 kDa | T cell function | 1.72 | 1.00 |
| CD3D | CD3D antigen, delta polypeptide (TiT3 complex) | T cell function | 1.71 | 1.10 |
| MELK | maternal embryonic leucine zipper kinase | stem cell renewal, cell cycle progression, | 1.71 | 1.08 |
| | | and pre-mRNA splicing | | |
| PRDX2 | peroxiredoxin 2 | potential antioxidant and antiviral. | 1.67 | −1.02 |
| RACGAP1 | Rac GTPase activating protein 1 | signaling | 1.67 | 1.00 |
| ITGA4 | integrin, alpha 4(antigen CD49D, alpha 4 subunit of | Immune/inflammatory processes | 1.66 | 1.07 |
| | VLA-4 receptor) | | | |
| PSIP1 | PC4 and SFRS1 interacting protein 1 | transcription | 1.66 | 1.01 |
| TACC3 | transforming, acidic coiled-coil containing protein 3 | centrosome/mitotic spindle apparatus | 1.63 | 1.10 |
| CD2 | CD2 antigen (p50), sheep red blood cell receptor | immune cell mediator | 1.62 | 1.10 |
| BCCIP | BRCA2 and CDKN1A interacting protein | cell cycle, tumor suppression | 1.61 | −1.02 |
| OIP5 | Opa-interacting protein 5 | unknown, binds to bacterial protein | 1.60 | 1.05 |
| PRKDC | protein kinase, DNA-activated, catalytic polypeptide | DNA damage/DNA synthesis | 1.59 | 1.10 |
| HNRPUL1 | heterogeneous nuclear ribonucleoprotein U-like 1 | nuclear RNA-binding protein | 1.59 | −1.03 |
| PSCDBP | pleckstrin homology, Sec7 and coiled-coil domains, | cytokine inducible-scaffold protein | 1.58 | 1.01 |
| | binding protein | | | |
| IL21R | interleukin 21 receptor | proliferation and differentiation of immune cells. | 1.55 | 1.07 |
| PARP1 | ADP-ribosyltransferase (NAD+; poly (ADP-ribose) | cell differentiation, proliferation, and tumor | 1.54 | 1.07 |
| | polymerase) | transformation DNA damage response | | |
| LCK | lymphocyte-specific protein tyrosine kinase | T cell function/immune response | 1.53 | 1.09 |
| GPX7 | glutathione peroxidase 7 | oxidative stress response | 1.53 | 1.06 |
| RAD21 | RAD21 homolog (S. pombe) | DNA repair/mitosis | 1.53 | 1.03 |
| PTTG1 | pituitary tumor-transforming 1 | tumorigenic/chromatid separation | 1.52 | 1.10 |
| C6ORF149 | chromosome 6 open reading frame 149 | Unknown | 1.52 | 1.06 |
| SNRPD3 | small nuclear ribonucleoprotein D3 polypeptide 18 kDa | pre-mRNA splicing and small nuclear | 1.52 | 1.03 |
| | | ribonucleoprotein biogenesis | | |
| FYN | FYN oncogene related to SRC, FGR, YES | cell growth, immune cell signaling | 1.51 | 1.02 |

TABLE 6B
GENE EXPRESSION DIFFERENCES BETWEEN ASTHMA AND HEALTHY SUBJECTS IN RESPONSE TO ALLERGEN

| | | | AOS | WHV |
|---|---|---|---|---|
| | | | FOLD | FOLD |
| SYMBOL | DESCRIPTION | FUNCTION | CHANGE | CHANGE |
| GM2A | GM2 ganglioside activator | glycolipid transport | −2.05 | −1.02 |
| SLC36A1 | solute carrier family 36 (proton/amino acid symporter), | small amino acid transporter | −1.90 | 1.01 |
| | member 1 | | | |
| TM6SF1 | transmembrane 6 superfamily member 1 | Unknown | −1.75 | −1.16 |
| LCK | lymphocyte-specific protein tyrosine kinase | T cell function/immune response | −1.68 | 1.05 |
| PYGL | phosphorylase, glycogen; liver (Hers disease,) | glycogen breakdown | −1.68 | −1.10 |
| PLEKHB2 | pleckstrin homology domain containing, family B member 2 | vesicular proteins | −1.67 | 1.06 |
| CD84 | CD84 antigen (leukocyte antigen) | cell adhesion | −1.66 | −1.07 |
| GCHFR | GTP cyclohydrolase I feedback regulator | tetrahydrobiopterin biosynthesis | −1.65 | −1.03 |
| SORT1 | sortilin 1 | lysosomal trafficking | −1.65 | −1.04 |
| HLA-DQB1 | major histocompatibility complex, class II, DQ beta 1 | antigen presentation | −1.62 | −1.03 |
| SLCO2B1 | solute carrier organic anion transporter family, member 2B1 | organic anion transporting polypeptide | −1.60 | −1.00 |
| ZFYVE26 | zinc finger, FYVE domain containing 26 | Unknown | −1.59 | −1.02 |
| TLR4 | toll-like receptor 4 | immune signaling receptor | −1.56 | −1.01 |
| HLA-DMB | major histocompatibility complex, class II, DM beta | antigen presentation | −1.56 | −1.01 |
| RNF13 | ring finger protein 13 | Unknown | −1.56 | −1.08 |
| PRNP | prion protein (p27-30) | prion diseases/oxidative stress | −1.55 | −1.02 |
| GAS7 | growth arrest-specific 7 | neuronal differentiation | −1.53 | −1.10 |
| ATP6V1A | ATPase, H+ transporting, lysosomal 70 kDa, V1 subunit A | acidification of eukaryotic intracellular organelles | −1.52 | 1.02 |
| ATP6V0D1 | ATPase, H+ transporting, lysosomal 38 kDa, V0 subunit d | acidification of eukaryotic intracellular organelles | −1.51 | −1.09 |
| | isoform 1 | | | |

TABLE 7A
NODES MODULATED SIMILARLY BETWEEN ASTHMATICS AND HEALTHY VOLUNTEERS
Table 7a. 133 Nodes are modulated similarly in response to allergen in the Asthmatics and Healthy Volunteers.
Fold changes represent differences in expression of genes in the presence and absence of allergen
(AG) and with and without a cPLA2 inhibitor (cPLA2) (4-{3-[1-benzhydryl-5-chloro-2-(2-{[(2,6-
dimethylbenzyl)sulfonyl]amino}ethyl)-1H-indol-3-yl]propyl}benzoic acid) and are averaged
from the individual asthmatic (AOS) and healthy volunteers (WHV) changes. Affymetrix identification
numbers, gene names and descriptions along with the False Discovery Rate (FDR) are given.
The fourth column provides the FDR for the significance of the association of the gene with asthma in
PBMCs prior to culture (that is, untreated PBMCs). The FDR was calculated in Spotfire using the deltas
(changes in expression of allergen vs. no allergen) for each of the treatment groups.

| | | | FDR for | | |
|---|---|---|---|---|---|
| | | | association | | |
| | | | with asthma | FDR | |
| | | | in PBMC | AOS | Fold |
| Affymetrix | Gene | | prior to | vs. | Change |
| ID | Name | Gene description | culture | WHV | AOS AG |
| 201951_at | ALCAM | activated leukocyte cell | Probeset did | 0.532514 | −3.032486 |
| | | adhesion molecule | not pass | | |
| | | | filters in | | |
| | | | PBMC | | |
| | | | analysis | | |
| 207016_s_at | ALDH1A2 | aldehyde | Probeset did | 0.767309 | −2.558599 |
| | | dehydrogenase 1 | not pass | | |
| | | family, member A2 | filters in | | |
| | | | PBMC | | |
| | | | analysis | | |
| 212883_at | APOE | apolipoprotein E | Probeset did | 0.892054 | −1.687718 |
| | | | not pass | | |
| | | | filters in | | |
| | | | PBMC | | |
| | | | analysis | | |
| 202686_s_at | AXL | AXL receptor tyrosine | Probeset did | 0.685558 | −1.954341 |
| | | kinase | not pass | | |
| | | | filters in | | |
| | | | PBMC | | |
| | | | analysis | | |
| 202094_at | BIRC5 | baculoviral IAP repeat- | Probeset did | 0.830323 | 1.8052641 |
| | | containing 5 (survivin) | not pass | | |
| | | | filters in | | |
| | | | PBMC | | |
| | | | analysis | | |
| 210735_s_at | CA12 | carbonic anhydrase XII | Probeset did | 0.814103 | 1.4502893 |
| | | | not pass | | |
| | | | filters in | | |
| | | | PBMC | | |
| | | | analysis | | |
| 207533_at | CCL1 | chemokine (C-C motif) | Probeset did | 0.826204 | 1.8809476 |
| | | ligand 1 | not pass | | |
| | | | filters in | | |
| | | | PBMC | | |
| | | | analysis | | |
| 216714_at | CCL13 | chemokine (C-C motif) | Probeset did | 0.744378 | −2.341058 |
| | | ligand 13 | not pass | | |
| | | | filters in | | |
| | | | PBMC | | |
| | | | analysis | | |
| 32128_at | CCL18 | chemokine (C-C motif) | Probeset did | 0.912661 | 2.6494141 |
| | | ligand 18 (pulmonary | not pass | | |
| | | and activation- | filters in | | |
| | | regulated) | PBMC | | |
| | | | analysis | | |
| 209924_at | CCL18 | chemokine (C-C motif) | Probeset did | 0.74245 | 2.6569649 |
| | | ligand 18 (pulmonary | not pass | | |
| | | and activation- | filters in | | |
| | | regulated) | PBMC | | |
| | | | analysis | | |
| 221463_at | CCL24 | chemokine (C-C motif) | Probeset did | 0.775846 | 1.5409421 |
| | | ligand 24 | not pass | | |
| | | | filters in | | |
| | | | PBMC | | |
| | | | analysis | | |
| 208712_at | CCND1 | cyclin D1 (PRAD1: | Probeset did | 0.611403 | −2.415046 |
| | | parathyroid | not pass | | |
| | | adenomatosis 1) | filters in | | |
| | | | PBMC | | |
| | | | analysis | | |
| 205046_at | CENPE | centromere protein E, | Probeset did | 0.77132 | 1.7625676 |
| | | 312 kDa | not pass | | |
| | | | filters in | | |
| | | | PBMC | | |
| | | | analysis | | |
| 213415_at | CLIC2 | chloride intracellular | Probeset did | 0.668499 | −2.043661 |
| | | channel 2 | not pass | | |
| | | | filters in | | |
| | | | PBMC | | |
| | | | analysis | | |
| 221881_s_at | CLIC4 | chloride intracellular | Probeset did | 0.910319 | −1.602364 |
| | | channel 4 | not pass | | |
| | | | filters in | | |
| | | | PBMC | | |
| | | | analysis | | |
| 210571_s_at | CMAH | cytidine | Probeset did | 0.74972 | 2.2158585 |
| | | monophosphate-N- | not pass | | |
| | | acetylneuraminic acid | filters in | | |
| | | hydroxylase (CMP-N- | PBMC | | |
| | | acetylneuraminate | analysis | | |
| | | monooxygenase) | | | |
| 221900_at | COL8A2 | collagen, type VIII, | Probeset did | 0.580426 | −2.491684 |
| | | alpha 2 | not pass | | |
| | | | filters in | | |
| | | | PBMC | | |
| | | | analysis | | |
| 205676_at | CYP27B1 | cytochrome P450, | Probeset did | 0.988756 | −2.13515 |
| | | family 27, subfamily B, | not pass | | |
| | | polypeptide 1 | filters in | | |
| | | | PBMC | | |
| | | | analysis | | |
| 203716_s_at | DPP4 | dipeptidylpeptidase 4 | Probeset did | 0.862769 | 1.8495199 |
| | | (CD26, adenosine | not pass | | |
| | | deaminase complexing | filters in | | |
| | | protein 2) | PBMC | | |
| | | | analysis | | |
| 203355_s_at | EFA6R | ADP-ribosylation factor | Probeset did | 0.774701 | −2.536485 |
| | | guanine nucleotide | not pass | | |
| | | factor 6 | filters in | | |
| | | | PBMC | | |
| | | | analysis | | |
| 219232_s_at | EGLN3 | egl nine homolog 3 (C. elegans) | Probeset did | 0.721743 | −2.146189 |
| | | | not pass | | |
| | | | filters in | | |
| | | | PBMC | | |
| | | | analysis | | |
| 203980_at | FABP4 | fatty acid binding | Probeset did | 0.721017 | −1.602005 |
| | | protein 4, adipocyte | not pass | | |
| | | | filters in | | |
| | | | PBMC | | |
| | | | analysis | | |
| 219525_at | FLJ10847 | hypothetical protein | Probeset did | 0.540165 | −2.170318 |
| | | FLJ10847 | not pass | | |
| | | | filters in | | |
| | | | PBMC | | |
| | | | analysis | | |
| 218417_s_at | FLJ20489 | hypothetical protein | Probeset did | 0.701782 | −1.933443 |
| | | FLJ20489 | not pass | | |
| | | | filters in | | |
| | | | PBMC | | |
| | | | analysis | | |
| 216442_x_at | FN1 | fibronectin 1 | Probeset did | 0.932348 | −23.65214 |
| | | | not pass | | |
| | | | filters in | | |
| | | | PBMC | | |
| | | | analysis | | |
| 212464_s_at | FN1 | fibronectin 1 | Probeset did | 0.916551 | −28.10718 |
| | | | not pass | | |
| | | | filters in | | |
| | | | PBMC | | |
| | | | analysis | | |
| 210495_x_at | FN1 | fibronectin 1 | Probeset did | 0.925963 | −27.19577 |
| | | | not pass | | |
| | | | filters in | | |
| | | | PBMC | | |
| | | | analysis | | |
| 211719_x_at | FN1 | fibronectin 1 | Probeset did | 0.962387 | −32.51561 |
| | | | not pass | | |
| | | | filters in | | |
| | | | PBMC | | |
| | | | analysis | | |
| 218885_s_at | GALNT12 | UDP-N-acetyl-alpha-D- | Probeset did | 0.809143 | −2.735878 |
| | | galactosamine:polypeptide | not pass | | |
| | | N- | filters in | | |
| | | acetylgalactosaminyltransferase | PBMC | | |
| | | 12 (GalNAc- | analysis | | |
| | | T12) | | | |
| 204472_at | GEM | GTP binding protein | Probeset did | 0.933924 | −1.636557 |
| | | overexpressed in | not pass | | |
| | | skeletal muscle | filters in | | |
| | | | PBMC | | |
| | | | analysis | | |
| 204836_at | GLDC | glycine dehydrogenase | Probeset did | 0.594954 | 2.007039 |
| | | (decarboxylating; | not pass | | |
| | | glycine decarboxylase, | filters in | | |
| | | glycine cleavage | PBMC | | |
| | | system protein P) | analysis | | |
| 204983_s_at | GPC4 | glypican 4 | Probeset did | 0.664635 | −2.795807 |
| | | | not pass | | |
| | | | filters in | | |
| | | | PBMC | | |
| | | | analysis | | |
| 204984_at | GPC4 | glypican 4 | Probeset did | 0.791915 | −3.01539 |
| | | | not pass | | |
| | | | filters in | | |
| | | | PBMC | | |
| | | | analysis | | |
| 215942_s_at | GTSE1 | G-2 and S-phase | Probeset did | 0.620066 | 1.5002875 |
| | | expressed 1 | not pass | | |
| | | | filters in | | |
| | | | PBMC | | |
| | | | analysis | | |
| 205919_at | HBE1 | hemoglobin, epsilon 1 | Probeset did | 0.662634 | 2.1024502 |
| | | | not pass | | |
| | | | filters in | | |
| | | | PBMC | | |
| | | | analysis | | |
| 216876_s_at | IL17 | interleukin 17 (cytotoxic | Probeset did | 0.693458 | 2.8266288 |
| | | T-lymphocyte- | not pass | | |
| | | associated serine | filters in | | |
| | | esterase 8) | PBMC | | |
| | | | analysis | | |
| 206295_at | IL18 | interleukin 18 | Probeset did | 0.942048 | −1.861258 |
| | | (interferon-gamma- | not pass | | |
| | | inducing factor) | filters in | | |
| | | | PBMC | | |
| | | | analysis | | |
| 221165_s_at | IL22 | interleukin 22 | Probeset did | 0.977658 | 2.2512258 |
| | | | not pass | | |
| | | | filters in | | |
| | | | PBMC | | |
| | | | analysis | | |
| 221111_at | IL26 | interleukin 26 | Probeset did | 0.543821 | 2.5530936 |
| | | | not pass | | |
| | | | filters in | | |
| | | | PBMC | | |
| | | | analysis | | |
| 208193_at | IL9 | interleukin 9 | Probeset did | 0.791989 | 2.3466712 |
| | | | not pass | | |
| | | | filters in | | |
| | | | PBMC | | |
| | | | analysis | | |
| 210029_at | INDO | indoleamine-pyrrole 2,3 | Probeset did | 0.907565 | 2.2512245 |
| | | dioxygenase | not pass | | |
| | | | filters in | | |
| | | | PBMC | | |
| | | | analysis | | |
| 210036_s_at | KCNH2 | potassium voltage- | Probeset did | 0.821524 | 1.7987362 |
| | | gated channel, | not pass | | |
| | | subfamily H (eag- | filters in | | |
| | | related), member 2 | PBMC | | |
| | | | analysis | | |
| 205051_s_at | KIT | v-kit Hardy-Zuckerman | Probeset did | 0.894949 | 1.7209263 |
| | | 4 feline sarcoma viral | not pass | | |
| | | oncogene homolog | filters in | | |
| | | | PBMC | | |
| | | | analysis | | |
| 217975_at | LOC51186 | pp21 homolog | Probeset did | 0.85398 | −1.591638 |
| | | | not pass | | |
| | | | filters in | | |
| | | | PBMC | | |
| | | | analysis | | |
| 200784_s_at | LRP1 | low density lipoprotein- | Probeset did | 0.971462 | −1.897666 |
| | | related protein 1 (alpha- | not pass | | |
| | | 2-macroglobulin | filters in | | |
| | | receptor) | PBMC | | |
| | | | analysis | | |
| 204580_at | MMP12 | matrix | Probeset did | 0.626473 | −2.041327 |
| | | metalloproteinase 12 | not pass | | |
| | | (macrophage elastase) | filters in | | |
| | | | PBMC | | |
| | | | analysis | | |
| 201069_at | MMP2 | matrix | Probeset did | 0.633118 | −2.406511 |
| | | metalloproteinase 2 | not pass | | |
| | | (gelatinase A, 72 kDa | filters in | | |
| | | gelatinase, 72 kDa type | PBMC | | |
| | | IV collagenase) | analysis | | |
| 208422_at | MSR1 | macrophage scavenger | Probeset did | 0.978988 | −1.504434 |
| | | receptor 1 | not pass | | |
| | | | filters in | | |
| | | | PBMC | | |
| | | | analysis | | |
| 201710_at | MYBL2 | v-myb myeloblastosis | Probeset did | 0.942445 | 2.033041 |
| | | viral oncogene homolog | not pass | | |
| | | (avian)-like 2 | filters in | | |
| | | | PBMC | | |
| | | | analysis | | |
| 205085_at | ORC1L | origin recognition | Probeset did | 0.773454 | 1.6873183 |
| | | complex, subunit 1-like | not pass | | |
| | | (yeast) | filters in | | |
| | | | PBMC | | |
| | | | analysis | | |
| 201397_at | PHGDH | phosphoglycerate | Probeset did | 0.754266 | 1.5344581 |
| | | dehydrogenase | not pass | | |
| | | | filters in | | |
| | | | PBMC | | |
| | | | analysis | | |
| 221061_at | PKD2L1 | polycystic kidney | Probeset did | 0.726371 | −1.419074 |
| | | disease 2-like 1 | not pass | | |
| | | | filters in | | |
| | | | PBMC | | |
| | | | analysis | | |
| 203997_at | PTPN3 | protein tyrosine | Probeset did | 0.593356 | 2.4399751 |
| | | phosphatase, non- | not pass | | |
| | | receptor type 3 | filters in | | |
| | | | PBMC | | |
| | | | analysis | | |
| 206392_s_at | RARRES1 | retinoic acid receptor | Probeset did | 0.992022 | −2.677175 |
| | | responder (tazarotene | not pass | | |
| | | induced) 1 | filters in | | |
| | | | PBMC | | |
| | | | analysis | | |
| 206851_at | RNASE3 | ribonuclease, RNase A | Probeset did | 0.956775 | 1.8865142 |
| | | family, 3 (eosinophil | not pass | | |
| | | cationic protein) | filters in | | |
| | | | PBMC | | |
| | | | analysis | | |
| 212912_at | RPS6KA2 | ribosomal protein S6 | Probeset did | 0.938059 | −1.905299 |
| | | kinase, 90 kDa, | not pass | | |
| | | polypeptide 2 | filters in | | |
| | | | PBMC | | |
| | | | analysis | | |
| 214507_s_at | RRP4 | homolog of Yeast RRP4 | Probeset did | 0.725234 | 1.8746799 |
| | | (ribosomal RNA | not pass | | |
| | | processing 4), 3′-5′- | filters in | | |
| | | exoribonuclease | PBMC | | |
| | | | analysis | | |
| 201427_s_at | SEPP1 | selenoprotein P, | Probeset did | 0.593585 | −5.300337 |
| | | plasma, 1 | not pass | | |
| | | | filters in | | |
| | | | PBMC | | |
| | | | analysis | | |
| 202628_s_at | SERPINE1 | serine (or cysteine) | Probeset did | 0.945562 | −1.890671 |
| | | proteinase inhibitor, | not pass | | |
| | | clade E (nexin, | filters in | | |
| | | plasminogen activator | PBMC | | |
| | | inhibitor type 1), | analysis | | |
| | | member 1 | | | |
| 202627_s_at | SERPINE1 | serine (or cysteine) | Probeset did | 0.736757 | −1.976537 |
| | | proteinase inhibitor, | not pass | | |
| | | clade E (nexin, | filters in | | |
| | | plasminogen activator | PBMC | | |
| | | inhibitor type 1), | analysis | | |
| | | member 1 | | | |
| 204430_s_at | SLC2A5 | solute carrier family 2 | Probeset did | 0.72425 | −1.968895 |
| | | (facilitated | not pass | | |
| | | glucose/fructose | filters in | | |
| | | transporter), member 5 | PBMC | | |
| | | | analysis | | |
| 202752_x_at | SLC7A8 | solute carrier family 7 | Probeset did | 0.95983 | −2.258179 |
| | | (cationic amino acid | not pass | | |
| | | transporter, y+ system), | filters in | | |
| | | member 8 | PBMC | | |
| | | | analysis | | |
| 220358_at | SNFT | Jun dimerization protein | Probeset did | 0.785415 | 3.4061381 |
| | | p21SNFT | not pass | | |
| | | | filters in | | |
| | | | PBMC | | |
| | | | analysis | | |
| 205342_s_at | SULT1C1 | sulfotransferase family, | Probeset did | 0.95487 | −2.032652 |
| | | cytosolic, 1C, member 1 | not pass | | |
| | | | filters in | | |
| | | | PBMC | | |
| | | | analysis | | |
| 201148_s_at | TIMP3 | tissue inhibitor of | Probeset did | 0.835235 | −3.263961 |
| | | metalloproteinase 3 | not pass | | |
| | | (Sorsby fundus | filters in | | |
| | | dystrophy, | PBMC | | |
| | | pseudoinflammatory) | analysis | | |
| 206026_s_at | TNFAIP6 | tumor necrosis factor, | Probeset did | 0.899344 | 1.6945987 |
| | | alpha-induced protein 6 | not pass | | |
| | | | filters in | | |
| | | | PBMC | | |
| | | | analysis | | |
| 206025_s_at | TNFAIP6 | tumor necrosis factor, | Probeset did | 0.942043 | 1.6408898 |
| | | alpha-induced protein 6 | not pass | | |
| | | | filters in | | |
| | | | PBMC | | |
| | | | analysis | | |
| 205890_s_at | UBD | ubiquitin D | Probeset did | 0.953893 | −1.64562 |
| | | | not pass | | |
| | | | filters in | | |
| | | | PBMC | | |
| | | | analysis | | |
| 214038_at | UNK_AI984980 | Consensus includes | Probeset did | 0.523197 | 1.5167568 |
| | | gb: AI984980 /FEA = EST | not pass | | |
| | | /DB_XREF = gi: 5812257 | filters in | | |
| | | /DB_XREF = est: wr88g11.x1 | PBMC | | |
| | | /CLONE = IMAGE: 2494820 | analysis | | |
| | | /UG = Hs.271387 | | | |
| | | small inducible cytokine | | | |
| | | subfamily A (Cys-Cys), | | | |
| | | member 8 (monocyte | | | |
| | | chemotactic protein 2) | | | |
| | | /FL = gb: NM_005623.1 | | | |
| 204058_at | UNK_AL049699 | Consensus includes | Probeset did | 0.754266 | −1.813519 |
| | | gb: AL049699 | not pass | | |
| | | /DEF = Human DNA | filters in | | |
| | | sequence from clone | PBMC | | |
| | | 747H23 on | analysis | | |
| | | chromosome 6q13-15. | | | |
| | | Contains the 3 part of | | | |
| | | the ME1 gene for malic | | | |
| | | enzyme 1, soluble | | | |
| | | (NADP-dependent malic | | | |
| | | enzyme, malate | | | |
| | | oxidoreductase, EC | | | |
| | | 1.1.1.40), a novel gene | | | |
| | | and the 5 part of the | | | |
| | | gene for N-acetylgl . . . | | | |
| | | /FEA = mRNA_3 | | | |
| | | /DB_XREF = gi: 5419832 | | | |
| | | /UG = Hs.14732 malic | | | |
| | | enzyme 1, NADP(+)- | | | |
| | | dependent, cytosolic | | | |
| | | /FL = gb: NM_002395.2 | | | |
| 204517_at | UNK_BE962749 | Consensus includes | Probeset did | 0.708065 | −2.279351 |
| | | gb: BE962749 | not pass | | |
| | | /FEA = EST | filters in | | |
| | | /DB_XREF = gi: 11765968 | PBMC | | |
| | | /DB_XREF = est: 601656143R1 | analysis | | |
| | | /CLONE = IMAGE: 3855754 | | | |
| | | /UG = Hs.110364 | | | |
| | | peptidylprolyl isomerase | | | |
| | | C (cyclophilin C) | | | |
| | | /FL = gb: BC002678.1 | | | |
| | | gb: NM_000943.1 | | | |
| 216905_s_at | UNK_U20428 | Consensus includes | Probeset did | 0.680738 | −1.826394 |
| | | gb: U20428.1 | not pass | | |
| | | /DEF = Human SNC19 | filters in | | |
| | | mRNA sequence. | PBMC | | |
| | | /FEA = mRNA | analysis | | |
| | | /DB_XREF = gi: 1890631 | | | |
| | | /UG = Hs.56937 | | | |
| | | suppression of | | | |
| | | tumorigenicity 14 (colon | | | |
| | | carcinoma, matriptase, | | | |
| | | epithin) | | | |
| 219753_at | STAG3 | stromal antigen 3 | 0.973347673 | 0.694604 | 1.860892 |
| 212334_at | GNS | glucosamine (N-acetyl)- | 0.942210568 | 0.616289 | −1.815407 |
| | | 6-sulfatase (Sanfilippo | | | |
| | | disease IIID) | | | |
| 203066_at | GALNAC4S- | B cell RAG associated | 0.910736959 | 0.805498 | −1.795781 |
| | 6ST | protein | | | |
| 218638_s_at | SPON2 | spondin 2, extracellular | 0.903622447 | 0.978555 | −2.034414 |
| | | matrix protein | | | |
| 212185_x_at | MT2A | metallothionein 2A | 0.807148264 | 0.786382 | 2.0273731 |
| 208161_s_at | ABCC3 | ATP-binding cassette, | 0.798684288 | 0.571886 | −1.991359 |
| | | sub-family C | | | |
| | | (CFTR/MRP), member 3 | | | |
| 210776_x_at | TCF3 | transcription factor 3 | 0.710816326 | 0.704463 | 1.6426719 |
| | | (E2A immunoglobulin | | | |
| | | enhancer binding | | | |
| | | factors E12/E47) | | | |
| 207543_s_at | P4HA1 | procollagen-proline, 2- | 0.629008685 | 0.61991 | −1.743072 |
| | | oxoglutarate 4- | | | |
| | | dioxygenase (proline 4- | | | |
| | | hydroxylase), alpha | | | |
| | | polypeptide I | | | |
| 202888_s_at | ANPEP | alanyl (membrane) | 0.610713096 | 0.639795 | −1.707372 |
| | | aminopeptidase | | | |
| | | (aminopeptidase N, | | | |
| | | aminopeptidase M, | | | |
| | | microsomal | | | |
| | | aminopeptidase, CD13, | | | |
| | | p150) | | | |
| 216092_s_at | SLC7A8 | solute carrier family 7 | 0.561081345 | 0.906849 | −1.759565 |
| | | (cationic amino acid | | | |
| | | transporter, y+ system), | | | |
| | | member 8 | | | |
| 209716_at | CSF1 | colony stimulating factor | 0.520999064 | 0.982971 | −1.795749 |
| | | 1 (macrophage) | | | |
| 208450_at | LGALS2 | lectin, galactoside- | 0.515832328 | 0.599434 | −1.845249 |
| | | binding, soluble, 2 | | | |
| | | (galectin 2) | | | |
| 214020_x_at | ITGB5 | integrin, beta 5 | 0.478567878 | 0.975385 | −1.956575 |
| 219066_at | MDS018 | hypothetical protein | 0.435088764 | 0.869358 | 1.628528 |
| | | MDS018 | | | |
| 205695_at | SDS | serine dehydratase | 0.353192135 | 0.674283 | −1.934026 |
| 217738_at | PBEF1 | pre-B-cell colony | 0.313619686 | 0.641074 | 1.9006161 |
| | | enhancing factor 1 | | | |
| 212187_x_at | PTGDS | prostaglandin D2 | 0.293745571 | 0.967135 | −2.126834 |
| | | synthase 21 kDa (brain) | | | |
| 210354_at | UNK_M29383 | gb: M29383.1 | 0.250248685 | 0.915462 | 2.0276129 |
| | | /DEF = Human | | | |
| | | interferon-gamma | | | |
| | | (HuIFN-gamma) mRNA, | | | |
| | | complete cds. | | | |
| | | /FEA = mRNA | | | |
| | | /DB_XREF = gi: 186514 | | | |
| | | /UG = Hs.856 interferon, | | | |
| | | gamma | | | |
| | | /FL = gb: NM_000619.1 | | | |
| | | gb: M29383.1 | | | |
| 209122_at | ADFP | adipose differentiation- | 0.182403199 | 0.868713 | −1.577006 |
| | | related protein | | | |
| 203832_at | SNRPF | small nuclear | 0.125966767 | 0.670508 | 1.7312364 |
| | | ribonucleoprotein | | | |
| | | polypeptide F | | | |
| 202499_s_at | SLC2A3 | solute carrier family 2 | 0.121673103 | 0.872288 | −1.865209 |
| | | (facilitated glucose | | | |
| | | transporter), member 3 | | | |
| 204103_at | CCL4 | chemokine (C-C motif) | 0.113108027 | 0.814256 | −1.60879 |
| | | ligand 4 | | | |
| 204614_at | SERPINB2 | serine (or cysteine) | 0.110994689 | 0.616289 | 1.7242525 |
| | | proteinase inhibitor, | | | |
| | | clade B (ovalbumin), | | | |
| | | member 2 | | | |
| 202498_s_at | SLC2A3 | solute carrier family 2 | 0.109688241 | 0.896496 | −1.857044 |
| | | (facilitated glucose | | | |
| | | transporter), member 3 | | | |
| 202973_x_at | FAM13A1 | family with sequence | 0.094489621 | 0.762119 | −1.801912 |
| | | similarity 13, member | | | |
| | | A1 | | | |
| 217047_s_at | FAM13A1 | family with sequence | 0.08632235 | 0.994143 | −1.59603 |
| | | similarity 13, member | | | |
| | | A1 | | | |
| 208581_x_at | MT1X | metallothionein 1X | 0.085563142 | 0.614059 | 2.1266441 |
| 204661_at | CDW52 | CDW52 antigen | 0.076086442 | 0.672622 | −1.857272 |
| | | (CAMPATH-1 antigen) | | | |
| 219799_s_at | DHRS9 | dehydrogenase/reductase | 0.066617414 | 0.76671 | −1.971565 |
| | | (SDR family) | | | |
| | | member 9 | | | |
| 209774_x_at | CXCL2 | chemokine (C—X—C | 0.05587374 | 0.600417 | 1.7703482 |
| | | motif) ligand 2 | | | |
| 204446_s_at | ALOX5 | arachidonate 5- | 0.038848455 | 0.898388 | −1.846481 |
| | | lipoxygenase | | | |
| 204470_at | CXCL1 | chemokine (C—X—C | 0.035816644 | 0.684929 | 4.7978591 |
| | | motif) ligand 1 | | | |
| | | (melanoma growth | | | |
| | | stimulating activity, | | | |
| | | alpha) | | | |
| 217165_x_at | MT1F | metallothionein 1F | 0.029726467 | 0.616895 | 1.9602008 |
| | | (functional) | | | |
| 208792_s_at | CLU | clusterin (complement | 0.0296116 | 0.825087 | −1.744743 |
| | | lysis inhibitor, SP-40,40, | | | |
| | | sulfated glycoprotein 2, | | | |
| | | testosterone-repressed | | | |
| | | prostate message 2, | | | |
| | | apolipoprotein J) | | | |
| 203485_at | RTN1 | reticulon 1 | 0.029360475 | 0.974427 | −1.605297 |
| 208791_at | CLU | clusterin (complement | 0.017551767 | 0.785735 | −2.380179 |
| | | lysis inhibitor, SP-40,40, | | | |
| | | sulfated glycoprotein 2, | | | |
| | | testosterone-repressed | | | |
| | | prostate message 2, | | | |
| | | apolipoprotein J) | | | |
| 218872_at | TSC | hypothetical protein | 0.014557527 | 0.925151 | 1.6803904 |
| | | FLJ20607 | | | |
| 205047_s_at | ASNS | asparagine synthetase | 0.011086747 | 0.65646 | 2.380442 |
| 215118_s_at | MGC27165 | hypothetical protein | 0.003988005 | 0.878327 | 1.5585085 |
| | | MGC27165 | | | |
| 201656_at | ITGA6 | integrin, alpha 6 | 0.003389493 | 0.92954 | −1.669457 |
| 202856_s_at | SLC16A3 | solute carrier family 16 | 0.001435654 | 0.734306 | −1.711334 |
| | | (monocarboxylic acid | | | |
| | | transporters), member 3 | | | |
| 202283_at | SERPINF1 | serine (or cysteine) | 0.000643342 | 0.766584 | −4.917846 |
| | | proteinase inhibitor, | | | |
| | | clade F (alpha-2 | | | |
| | | antiplasmin, pigment | | | |
| | | epithelium derived | | | |
| | | factor), member 1 | | | |
| 205997_at | ADAM28 | a disintegrin and | 0.000493506 | 0.814705 | −2.04426 |
| | | metalloproteinase | | | |
| | | domain 28 | | | |
| 214581_x_at | UNK_BE568134 | Consensus includes | 7.71157E−05 | 0.945428 | −1.899264 |
| | | gb: BE568134 | | | |
| | | /FEA = EST | | | |
| | | /DB_XREF = gi: 9811854 | | | |
| | | /DB_XREF = est: 601341661F1 | | | |
| | | /CLONE = IMAGE: 3683823 | | | |
| | | /UG = Hs.159651 | | | |
| | | death receptor 6 | | | |
| | | /FL = gb: AF068868.1 | | | |
| | | gb: NM_014452.1 | | | |
| 202934_at | HK2 | hexokinase 2 | 3.89927E−05 | 0.788497 | −1.650883 |
| 217983_s_at | RNASET2 | ribonuclease T2 | 3.36876E−05 | 0.620557 | −1.968597 |
| 210889_s_at | FCGR2B | Fc fragment of IgG, low | 3.15176E−05 | 0.734045 | −2.326139 |
| | | affinity IIb, receptor for | | | |
| | | (CD32) | | | |
| 207850_at | CXCL3 | chemokine (C—X—C | 1.39743E−05 | 0.794984 | 1.7384592 |
| | | motif) ligand 3 | | | |
| 219434_at | TREM1 | triggering receptor | 2.17273E−06 | 0.910593 | −2.182721 |
| | | expressed on myeloid | | | |
| | | cells 1 | | | |
| 211506_s_at | UNK_AF043337 | gb: AF043337.1 | 6.26877E−07 | 0.694213 | 5.5162626 |
| | | /DEF = Homo sapiens | | | |
| | | interleukin 8 C-terminal | | | |
| | | variant (IL8) mRNA, | | | |
| | | complete cds. | | | |
| | | /FEA = mRNA /GEN = IL8 | | | |
| | | /PROD = interleukin 8 C- | | | |
| | | terminal variant | | | |
| | | /DB_XREF = gi: 12641914 | | | |
| | | /UG = Hs.624 | | | |
| | | interleukin 8 | | | |
| | | /FL = gb: AF043337.1 | | | |
| 203949_at | MPO | myeloperoxidase | 5.55649E−07 | 0.617534 | 2.0142114 |
| 206871_at | ELA2 | elastase 2, neutrophil | 1.40865E−07 | 0.704542 | 3.2848197 |
| 205898_at | CX3CR1 | chemokine (C—X3—C | 8.05971E−08 | 0.726371 | −1.539807 |
| | | motif) receptor 1 | | | |
| 209116_x_at | HBB | hemoglobin, beta | 7.98238E−09 | 0.54345 | 3.731341 |
| 217232_x_at | UNK_AF059180 | Consensus includes | 1.17022E−09 | 0.650843 | 3.2357142 |
| | | gb: AF059180 | | | |
| | | /DEF = Homo sapiens | | | |
| | | mutant beta-globin | | | |
| | | (HBB) gene, complete | | | |
| | | cds /FEA = mRNA | | | |
| | | /DB_XREF = gi: 4837722 | | | |
| | | /UG = Hs.155376 | | | |
| | | hemoglobin, beta | | | |
| 211696_x_at | HBB | hemoglobin, beta | 2.2979E−10 | 0.650195 | 3.2154588 |
| 205568_at | AQP9 | aquaporin 9 | 1.98427E−10 | 0.808099 | −1.659623 |
| 202859_x_at | IL8 | interleukin 8 | 6.56808E−11 | 0.715155 | 3.859481 |
| 203646_at | FDX1 | ferredoxin 1 | 6.20748E−11 | 0.899666 | −1.521268 |
| 205624_at | CPA3 | carboxypeptidase A3 | 1.85576E−12 | 0.896437 | 1.8544075 |
| | | (mast cell) | | | |
| 206207_at | CLC | Charcot-Leyden crystal | 0 | 0.76011 | 2.1381819 |
| | | protein | | | |

| | Fold | | Fold | | |
|---|---|---|---|---|---|
| | Change | Fold | Change | FDR AOS AG | FDR HV AG |
| | AOS AG + | Change | WHV AG + | vs AG + | vs AG + |
| Affymetrix | cPLA2 | WHV | cPLA2 | cPLA2 | cPLA2 |
| ID | inhibitor | AG | inhibitor | inhibitor | inhibitor |
| 201951_at | 1.194486 | −2.36 | 1.31808 | 0.034486 | 0.123591 |
| 207016_s_at | −1.09756 | −2.29 | −1.46369 | 0.343056 | 0.081988 |
| 212883_at | 1.109581 | −1.62 | 1.281126 | 0.196663 | 0.165955 |
| 202686_s_at | 1.066083 | −1.63 | 1.522625 | 0.686858 | 0.194435 |
| 202094_at | −1.22766 | 1.65 | −1.34124 | 0.011586 | 0.006499 |
| 210735_s_at | −1.31029 | 1.60 | −1.38875 | 0.002049 | 0.06248 |
| 207533_at | −1.07568 | 1.69 | 1.353353 | 0.655327 | 0.250557 |
| 216714_at | 1.226581 | −1.93 | 1.659363 | 0.296864 | 0.049489 |
| 32128_at | 1.180667 | 2.50 | −1.52188 | 0.115145 | 0.025587 |
| 209924_at | 1.147725 | 2.31 | −1.51576 | 0.083363 | 0.044326 |
| 221463_at | −1.49781 | 1.79 | −1.80123 | 0.000657 | 0.004856 |
| 208712_at | 1.103552 | −1.94 | 1.61239 | 0.289844 | 0.098125 |
| 205046_at | −1.24579 | 1.56 | −1.24276 | 0.009204 | 0.1579 |
| 213415_at | −1.04616 | −1.75 | −1.05169 | 0.762224 | 0.767056 |
| 221881_s_at | 1.279858 | −1.51 | 1.657655 | 0.010446 | 0.056488 |
| 210571_s_at | −1.32323 | 1.94 | −1.52645 | 0.00026 | 0.005581 |
| 221900_at | 1.122104 | −2.01 | 1.317966 | 0.215541 | 0.328459 |
| 205676_at | 1.547297 | −2.15 | 1.555581 | 1.53E−07 | 0.021087 |
| 203716_s_at | −1.77033 | 1.65 | −1.25129 | 8.05E−05 | 0.499764 |
| 203355_s_at | 1.228074 | −2.28 | 1.170581 | 0.006764 | 0.491483 |
| 219232_s_at | 1.076401 | −2.50 | 1.203241 | 0.425023 | 0.331154 |
| 203980_at | −1.5319 | −1.98 | −1.29026 | 0.000525 | 0.431737 |
| 219525_at | 1.102443 | −1.63 | −1.00462 | 0.585223 | 0.989734 |
| 218417_s_at | 1.380226 | −1.66 | 1.394938 | 0.001926 | 0.162145 |
| 216442_x_at | −1.19773 | −21.42 | −1.1466 | 0.341527 | 0.788253 |
| 212464_s_at | −1.29163 | −24.90 | −1.10096 | 0.228816 | 0.872769 |
| 210495_x_at | −1.349 | −24.60 | −1.0458 | 0.151302 | 0.938957 |
| 211719_x_at | −1.39669 | −34.34 | 1.005733 | 0.116755 | 0.992463 |
| 218885_s_at | 1.155005 | −2.43 | 1.455761 | 0.245509 | 0.095551 |
| 204472_at | −1.1651 | −1.58 | −1.02491 | 0.049535 | 0.870677 |
| 204836_at | −1.14958 | 1.70 | −1.4634 | 0.123987 | 0.029425 |
| 204983_s_at | 1.150933 | −2.32 | 1.289807 | 0.090876 | 0.099238 |
| 204984_at | 1.245818 | −2.65 | 1.186867 | 0.000128 | 0.35623 |
| 215942_s_at | −1.24904 | 1.76 | −1.29663 | 0.000525 | 0.112599 |
| 205919_at | −1.38008 | 2.74 | −1.4406 | 0.003121 | 0.071816 |
| 216876_s_at | −1.12668 | 2.33 | −1.1227 | 0.365377 | 0.622439 |
| 206295_at | 1.321242 | −1.93 | 1.568286 | 0.00436 | 0.020376 |
| 221165_s_at | −1.2413 | 2.28 | −1.28841 | 0.009821 | 0.199481 |
| 221111_at | −1.30364 | 1.88 | 1.191819 | 0.002032 | 0.394227 |
| 208193_at | −1.71258 | 2.00 | −1.38668 | 8.89E−06 | 0.166899 |
| 210029_at | 1.045322 | 2.07 | 1.131988 | 0.608878 | 0.562589 |
| 210036_s_at | −1.40252 | 1.61 | −1.33132 | 0.000217 | 0.048213 |
| 205051_s_at | −1.23229 | 1.61 | −1.03925 | 0.014597 | 0.848829 |
| 217975_at | 1.192856 | −1.52 | 1.324217 | 0.010004 | 0.010647 |
| 200784_s_at | 1.249344 | −1.93 | 1.34983 | 0.068934 | 0.253276 |
| 204580_at | 1.098056 | −2.82 | −1.00545 | 0.296739 | 0.981001 |
| 201069_at | 1.136363 | −1.99 | 1.44241 | 0.246669 | 0.083539 |
| 208422_at | −1.09609 | −1.53 | −1.08241 | 0.523497 | 0.742636 |
| 201710_at | −1.29502 | 1.97 | −1.35996 | 0.000289 | 0.09015 |
| 205085_at | −1.17369 | 1.54 | −1.2623 | 0.011075 | 0.077246 |
| 201397_at | −1.05576 | 1.66 | −1.19799 | 0.422299 | 0.332461 |
| 221061_at | 1.103101 | −1.68 | 1.518192 | 0.516597 | 0.11801 |
| 203997_at | −1.92286 | 1.92 | −1.2925 | 1.02E−08 | 0.117654 |
| 206392_s_at | 1.729958 | −2.66 | 1.449075 | 0.002816 | 0.167166 |
| 206851_at | −1.14919 | 1.81 | −1.13017 | 0.279815 | 0.609646 |
| 212912_at | 1.309167 | −1.83 | 1.551996 | 0.013626 | 0.02654 |
| 214507_s_at | −1.35437 | 1.59 | −1.32731 | 0.009621 | 0.140809 |
| 201427_s_at | 1.291422 | −3.54 | 1.461318 | 0.267167 | 0.430836 |
| 202628_s_at | 1.108425 | −1.95 | 1.282201 | 0.168037 | 0.121599 |
| 202627_s_at | 1.10505 | −1.72 | 1.109767 | 0.229838 | 0.536511 |
| 204430_s_at | 1.223762 | −2.39 | 1.139701 | 0.153883 | 0.613701 |
| 202752_x_at | 1.380448 | −2.32 | 1.324017 | 9.01E−05 | 0.409601 |
| 220358_at | −1.40523 | 2.98 | −1.32644 | 1.2E−06 | 0.026177 |
| 205342_s_at | 1.109368 | −1.98 | 1.241821 | 0.330554 | 0.365599 |
| 201148_s_at | −1.00757 | −2.96 | 1.223659 | 0.959606 | 0.541373 |
| 206026_s_at | −1.14377 | 1.79 | 1.120026 | 0.063621 | 0.668105 |
| 206025_s_at | −1.11014 | 1.68 | 1.083271 | 0.242753 | 0.708862 |
| 205890_s_at | −1.05956 | −1.59 | −1.44257 | 0.564154 | 0.032947 |
| 214038_at | 1.248648 | 2.03 | 1.154581 | 0.01263 | 0.429272 |
| 204058_at | 1.385748 | −1.61 | 1.409784 | 0.00074 | 0.019855 |
| 204517_at | 1.249643 | −1.98 | 1.365806 | 0.024698 | 0.086746 |
| 216905_s_at | 1.036943 | −1.55 | 1.184215 | 0.79049 | 0.571505 |
| 219753_at | −1.33381 | 1.66 | −1.3274 | 6.14E−05 | 0.057603 |
| 212334_at | 1.468742 | −1.59 | 1.612677 | 1.49E−08 | 0.002077 |
| 203066_at | 1.214463 | −1.96 | 1.220314 | 0.001078 | 0.246399 |
| 218638_s_at | 1.212651 | −2.01 | 1.784503 | 0.059898 | 0.026939 |
| 212185_x_at | 1.056131 | 1.88 | 1.341475 | 0.176972 | 0.003575 |
| 208161_s_at | 1.225897 | −2.40 | 1.870542 | 0.029743 | 0.053691 |
| 210776_x_at | −1.28049 | 1.81 | −1.31685 | 0.000341 | 0.046175 |
| 207543_s_at | 1.182753 | −1.56 | 1.082054 | 8.73E−05 | 0.561811 |
| 202888_s_at | 1.05077 | −1.51 | 1.127779 | 0.478211 | 0.098088 |
| 216092_s_at | 1.17594 | −1.71 | 1.285097 | 0.001371 | 0.036946 |
| 209716_at | −1.00031 | −1.78 | 1.472667 | 0.997293 | 0.059443 |
| 208450_at | 1.269638 | −2.42 | 1.303187 | 0.041378 | 0.339677 |
| 214020_x_at | 1.28944 | −1.93 | 1.389495 | 0.009742 | 0.158897 |
| 219066_at | −1.17432 | 1.55 | −1.25456 | 0.039059 | 0.182653 |
| 205695_at | 1.086934 | −1.65 | 1.384919 | 0.311965 | 0.01138 |
| 217738_at | −1.17003 | 1.73 | −1.26096 | 3.06E−05 | 0.026533 |
| 212187_x_at | 1.472903 | −2.18 | 1.579363 | 0.004038 | 0.175623 |
| 210354_at | −1.13947 | 2.14 | −1.17799 | 0.162615 | 0.332461 |
| 209122_at | −1.03065 | −1.52 | −1.16574 | 0.58268 | 0.272735 |
| 203832_at | −1.13853 | 1.56 | −1.29265 | 0.02854 | 0.056039 |
| 202499_s_at | 1.149577 | −1.75 | 1.191576 | 0.002101 | 0.135693 |
| 204103_at | 1.16661 | −1.49 | 1.246895 | 0.003359 | 0.046687 |
| 204614_at | −1.50805 | 1.38 | −1.11342 | 6.93E−05 | 0.719316 |
| 202498_s_at | 1.193857 | −1.78 | 1.191046 | 0.020838 | 0.233351 |
| 202973_x_at | 1.017986 | −1.65 | 1.025804 | 0.816343 | 0.91339 |
| 217047_s_at | 1.02414 | −1.59 | 1.04921 | 0.771583 | 0.700163 |
| 208581_x_at | 1.093885 | 1.87 | 1.41423 | 0.047423 | 0.002722 |
| 204661_at | −1.06016 | −1.70 | 1.127423 | 0.415015 | 0.396643 |
| 219799_s_at | −1.05817 | −1.76 | 1.075473 | 0.458273 | 0.673575 |
| 209774_x_at | 1.158335 | 2.17 | −1.33474 | 0.032435 | 0.077723 |
| 204446_s_at | 1.256275 | −1.77 | 1.218008 | 2.62E−06 | 0.101069 |
| 204470_at | −1.52427 | 3.96 | −1.52456 | 2.51E−06 | 0.064476 |
| 217165_x_at | 1.152098 | 1.71 | 1.53288 | 0.013599 | 0.002457 |
| 208792_s_at | 1.110358 | −1.92 | 1.639652 | 0.220377 | 0.022261 |
| 203485_at | 1.35223 | −1.58 | 1.69685 | 0.000454 | 0.022909 |
| 208791_at | 1.149908 | −2.87 | 1.94639 | 0.224127 | 0.021754 |
| 218872_at | −1.29159 | 1.62 | −1.40548 | 0.0004 | 0.031404 |
| 205047_s_at | −1.30014 | 2.09 | −1.6091 | 0.000266 | 0.05663 |
| 215118_s_at | −1.09986 | 1.47 | −1.14567 | 0.018385 | 0.320147 |
| 201656_at | 1.160335 | −1.73 | 1.294581 | 0.014601 | 0.056039 |
| 202856_s_at | 1.262425 | −1.58 | 1.217017 | 2.31E−08 | 0.056673 |
| 202283_at | 1.548686 | −4.05 | 1.47916 | 0.004679 | 0.298955 |
| 205997_at | −1.03317 | −2.33 | 1.151431 | 0.823077 | 0.576667 |
| 214581_x_at | 1.060318 | −1.84 | 1.095812 | 0.585438 | 0.735846 |
| 202934_at | 1.181042 | −1.53 | 1.193572 | 6.51E−05 | 0.120638 |
| 217983_s_at | 1.314501 | −1.76 | 1.312743 | 1.58E−09 | 0.020213 |
| 210889_s_at | 1.304462 | −2.06 | 1.189967 | 5.37E−05 | 0.164669 |
| 207850_at | −1.1809 | 1.55 | −1.17664 | 0.056724 | 0.522586 |
| 219434_at | −1.11503 | −2.32 | −1.34438 | 0.183067 | 0.133197 |
| 211506_s_at | −1.62649 | 4.64 | −1.91428 | 4.24E−08 | 0.012401 |
| 203949_at | −1.05214 | 1.65 | 1.05412 | 0.555877 | 0.798347 |
| 206871_at | 1.017106 | 2.50 | −1.01092 | 0.870156 | 0.964187 |
| 205898_at | 1.092203 | −1.74 | 1.321182 | 0.297024 | 0.166075 |
| 209116_x_at | −1.59284 | 2.63 | −1.54801 | 1.29E−07 | 0.010957 |
| 217232_x_at | −1.6188 | 2.63 | −1.50501 | 1.61E−07 | 0.013917 |
| 211696_x_at | −1.56195 | 2.62 | −1.49168 | 2.66E−07 | 0.011659 |
| 205568_at | 1.022156 | −1.55 | 1.193528 | 0.706516 | 0.287856 |
| 202859_x_at | −1.44102 | 4.37 | −1.69499 | 4.85E−09 | 0.016271 |
| 203646_at | 1.059586 | −1.59 | 1.330343 | 0.440803 | 0.014947 |
| 205624_at | −1.24093 | 1.94 | −1.28855 | 0.000358 | 0.021085 |
| 206207_at | −1.07065 | 1.89 | −1.2567 | 0.212718 | 0.008088 |

TABLE 7B
ALLERGEN SPECIFIC CHANGES IN PBMCS, ASTHMATICS VS. HEALTHY VOLUNTEERS

| | | | | | | | | Fold | |
|---|---|---|---|---|---|---|---|---|---|
| | | | FDR for | | | | Fold | Change | |
| | | | association | | | | Change | WHV | |
| | | | with asthma | FDR | AOS fold | WHV fold | AOS | Allergen | AOS FDR |
| | | | in PBMC | AOS | change | changes | Allergen | vs. | Allergen v |
| Affymetrix | | | prior to | vs. | Allergen | Allergen | vs. cPLA2 | cPLA2 | cPLA2 |
| ID | Gene | Gene Description | culture | WHV | vs. NT | vs. NT | inhibitor | inhibitor | inhibitor |
| 212041_at | ATP6V0D1 | ATPase, H+ | <1E−15 | 0.051 | −1.51 | −1.09 | 2.29154 | 1.16447 | 0.00000 |
| | | transporting, lysosomal | | | | | | | |
| | | 38 kDa, V0 subunit d | | | | | | | |
| | | isoform 1 | | | | | | | |
| 201487_at | CTSC | cathepsin C | <1E−15 | 0.047 | −1.76 | −1.14 | 2.79134 | 1.20832 | 0.00000 |
| 203358_s_at | EZH2 | enhancer of zeste | <1E−15 | 0.047 | 1.79 | 1.14 | −1.17995 | −1.18442 | 0.00189 |
| | | homolog 2 (Drosophila) | | | | | | | |
| 211953_s_at | KPNB3/RANBP5 | karyopherin (importin) | <1E−15 | 0.037 | 1.72 | 1.06 | −1.21228 | −1.15775 | 0.00051 |
| | | beta 3 | | | | | | | |
| 203041_s_at | LAMP2 | lysosomal-associated | <1E−15 | 0.049 | −1.83 | −1.30 | 2.54517 | 1.26180 | 0.00000 |
| | | membrane protein 2 | | | | | | | |
| 212522_at | PDE8A | phosphodiesterase 8A | <1E−15 | 0.050 | −1.41 | −1.52 | −1.01219 | 1.02185 | 0.95955 |
| 201779_s_at | RNF13 | ring finger protein 13 | <1E−15 | 0.039 | −1.56 | −1.08 | 2.62459 | 1.21231 | 0.00000 |
| 217865_at | RNF130 | ring finger protein 130 | <1E−15 | 0.037 | −1.69 | −1.12 | 2.54033 | 1.14174 | 0.00000 |
| 202690_s_at | SNRPD1 | small nuclear | <1E−15 | 0.051 | 1.71 | 1.23 | −1.11581 | −1.19856 | 0.00020 |
| | | ribonucleoprotein D1 | | | | | | | |
| | | polypeptide 16 kDa | | | | | | | |
| 202567_at | SNRPD3 | small nuclear | <1E−15 | 0.023 | 1.52 | 1.03 | −1.17059 | −1.05799 | 0.00012 |
| | | ribonucleoprotein D3 | | | | | | | |
| | | polypeptide 18 kDa | | | | | | | |
| 221060_s_at | TLR4 | toll-like receptor 4 | <1E−15 | 0.039 | −1.56 | −1.01 | 2.20767 | 1.05343 | 0.00392 |
| 203432_at | TMPO | thymopoietin | <1E−15 | 0.049 | 1.62 | 1.24 | −1.19599 | −1.14379 | 0.00001 |
| 203300_x_at | AP1S2 | adaptor-related protein | 2.59456E−14 | 0.039 | −1.79 | −1.16 | 2.53321 | 1.17271 | 0.00000 |
| | | complex 1, sigma 2 | | | | | | | |
| | | subunit | | | | | | | |
| 219892_at | TM6SF1 | transmembrane 6 | 8.08522E−13 | 0.041 | −1.75 | −1.16 | 2.39900 | 1.06590 | 0.00000 |
| | | superfamily member 1 | | | | | | | |
| 208694_at | PRKDC | protein kinase, DNA- | 5.65981E−12 | 0.039 | 1.59 | 1.10 | −1.14179 | −1.26604 | 0.00073 |
| | | activated, catalytic | | | | | | | |
| | | polypeptide | | | | | | | |
| 211067_s_at | GAS7 | growth arrest-specific 7 | 6.28242E−12 | 0.047 | −1.53 | −1.10 | 2.33986 | 1.14011 | 0.00001 |
| 214032_at | ZAP70 | zeta-chain (TCR) | 6.34092E−12 | 0.026 | 1.72 | 1.00 | −1.15588 | −1.08715 | 0.00007 |
| | | associated protein | | | | | | | |
| | | kinase 70 kDa | | | | | | | |
| 201403_s_at | MGST3 | microsomal glutathione | 8.85532E−12 | 0.050 | −1.75 | −1.25 | 2.30104 | 1.09760 | 0.00000 |
| | | S-transferase 3 | | | | | | | |
| 215049_x_at | CD163 | CD163 antigen | 1.01101E−10 | 0.037 | −3.71 | −1.69 | 4.67404 | 1.68205 | 0.00000 |
| 200608_s_at | RAD21 | RAD21 homolog | 1.1293E−10 | 0.037 | 1.53 | 1.03 | −1.14959 | −1.23691 | 0.00010 |
| | | (S. pombe) | | | | | | | |
| 211841_s_at | TNFRSF25 | tumor necrosis factor | 9.36378E−10 | 0.026 | 2.93 | 1.29 | −1.39366 | −1.20297 | 0.00012 |
| | | receptor superfamily, | | | | | | | |
| | | member 25 | | | | | | | |
| 202265_at | BMI1 | B lymphoma Mo-MLV | 1.25582E−09 | 0.051 | 1.84 | 1.17 | −1.17445 | −1.23177 | 0.00062 |
| | | insertion region (mouse) | | | | | | | |
| 200983_x_at | CD59 | CD59 antigen p18-20 | 1.74272E−09 | 0.039 | −1.67 | −1.18 | 2.48556 | 1.25375 | 0.00000 |
| | | (antigen identified by | | | | | | | |
| | | monoclonal antibodies | | | | | | | |
| | | 16.3A5, EJ16, EJ30, | | | | | | | |
| | | EL32 and G344) | | | | | | | |
| 202191_s_at | GAS7 | growth arrest-specific 7 | 1.91924E−09 | 0.039 | −1.97 | −1.14 | 2.40369 | 1.13967 | 0.00004 |
| 203828_s_at | NK4 | natural killer cell | 2.01811E−09 | 0.047 | 1.91 | 1.34 | −1.15371 | −1.18729 | 0.00252 |
| | | transcript 4 | | | | | | | |
| 203932_at | HLA-DMB | major histocompatibility | 3.62095E−09 | 0.039 | −1.56 | −1.01 | 2.37240 | 1.05527 | 0.00009 |
| | | complex, class II, DM | | | | | | | |
| | | beta | | | | | | | |
| 219505_at | CECR1 | cat eye syndrome | 7.13012E−09 | 0.041 | −2.23 | −1.46 | 2.62528 | 1.35558 | 0.00000 |
| | | chromosome region, | | | | | | | |
| | | candidate 1 | | | | | | | |
| 204214_s_at | RAB32 | RAB32, member RAS | 8.34896E−09 | 0.037 | −1.93 | −1.21 | 2.41821 | 1.22173 | 0.00000 |
| | | oncogene family | | | | | | | |
| 203645_s_at | CD163 | CD163 antigen | 1.35109E−08 | 0.051 | −3.53 | −1.68 | 4.64259 | 1.69001 | 0.00000 |
| 216041_x_at | GRN | granulin | 1.36513E−08 | 0.037 | −2.00 | −1.27 | 2.52809 | 1.33283 | 0.00000 |
| 201590_x_at | ANXA2 | annexin A2 | 2.04224E−08 | 0.039 | −1.69 | −1.27 | 2.34246 | 1.27323 | 0.00000 |
| 208821_at | SNRPB | small nuclear | 3.79588E−08 | 0.039 | 1.59 | 1.14 | −1.12036 | −1.09614 | 0.00002 |
| | | ribonucleoprotein | | | | | | | |
| | | polypeptides B and B1 | | | | | | | |
| 214882_s_at | SFRS2 | splicing factor, | 4.6263E−08 | 0.051 | 1.53 | 1.11 | −1.13297 | −1.09762 | 0.00003 |
| | | arginine/serine-rich 2 | | | | | | | |
| 218109_s_at | FLJ14153 | hypothetical protein | 5.32759E−08 | 0.039 | −1.79 | −1.29 | 2.70658 | 1.27421 | 0.00000 |
| | | FLJ14153 | | | | | | | |
| 210427_x_at | ANXA2 | annexin A2 | 6.08472E−08 | 0.041 | −1.65 | −1.19 | 2.38663 | 1.19875 | 0.00000 |
| 211284_s_at | GRN | granulin | 8.3996E−08 | 0.037 | −2.10 | −1.28 | 2.63841 | 1.42260 | 0.00000 |
| 202481_at | DHRS3 | dehydrogenase/reductase | 1.20441E−07 | 0.042 | −1.42 | −1.53 | −1.01990 | −1.06352 | 0.84564 |
| | | (SDR family) | | | | | | | |
| | | member 3 | | | | | | | |
| 213503_x_at | UNK_BE908217 | Consensus includes | 1.25853E−07 | 0.039 | −1.69 | −1.27 | 2.36565 | 1.26898 | 0.00000 |
| | | gb: BE908217 | | | | | | | |
| | | /FEA = EST | | | | | | | |
| | | /DB_XREF = gi: | | | | | | | |
| | | 10402569 | | | | | | | |
| | | /DB_XREF = est: | | | | | | | |
| | | 601500477F1 | | | | | | | |
| | | /CLONE = IMAGE: | | | | | | | |
| | | 3902323 | | | | | | | |
| | | /UG = Hs.217493 | | | | | | | |
| | | annexin A2 | | | | | | | |
| 200678_x_at | GRN | granulin | 2.11036E−07 | 0.050 | −1.86 | −1.24 | 2.49291 | 1.32328 | 0.00000 |
| 203470_s_at | PLEK | pleckstrin | 2.41613E−07 | 0.042 | −2.31 | −1.41 | 2.97376 | 1.49306 | 0.00000 |
| 208644_at | ADPRT/PARP1 | ADP-ribosyltransferase | 3.05285E−07 | 0.023 | 1.54 | 1.07 | −1.17537 | −1.11548 | 0.00008 |
| | | (NAD+; poly (ADP- | | | | | | | |
| | | ribose) polymerase) | | | | | | | |
| 201900_s_at | AKR1A1 | aldo-keto reductase | 3.67421E−07 | 0.050 | −1.51 | −1.11 | 2.26452 | 1.19824 | 0.00000 |
| | | family 1, member A1 | | | | | | | |
| | | (aldehyde reductase) | | | | | | | |
| 202990_at | PYGL | phosphorylase, | 5.28107E−07 | 0.037 | −1.68 | −1.10 | 2.56101 | 1.18218 | 0.00000 |
| | | glycogen; liver (Hers | | | | | | | |
| | | disease, glycogen | | | | | | | |
| | | storage disease type VI) | | | | | | | |
| 200701_at | NPC2 | Niemann-Pick disease, | 3.37605E−06 | 0.039 | −1.88 | −1.37 | 2.41822 | 1.25740 | 0.00000 |
| | | type C2 | | | | | | | |
| 201140_s_at | RAB5C | RAB5C, member RAS | 3.44299E−06 | 0.048 | −1.08 | −1.51 | 2.02059 | 1.49705 | 0.54943 |
| | | oncogene family | | | | | | | |
| 201555_at | MCM3 | MCM3 | 4.99887E−06 | 0.039 | 1.61 | 1.17 | −1.18568 | −1.23153 | 0.00000 |
| | | minichromosome | | | | | | | |
| | | maintenance deficient 3 | | | | | | | |
| | | (S. cerevisiae) | | | | | | | |
| 202200_s_at | SRPK1 | SFRS protein kinase 1 | 5.03527E−06 | 0.037 | 1.57 | 1.16 | −1.13473 | −1.21063 | 0.00001 |
| 208949_s_at | LGALS3 | lectin, galactoside- | 5.54361E−06 | 0.037 | −1.77 | −1.36 | 2.37974 | 1.17306 | 0.00000 |
| | | binding, soluble, 3 | | | | | | | |
| | | (galectin 3) | | | | | | | |
| 210538_s_at | BIRC3 | baculoviral IAP repeat- | 6.35962E−06 | 0.051 | 1.60 | 1.16 | −1.23678 | −1.27670 | 0.00000 |
| | | containing 3 | | | | | | | |
| 209555_s_at | CD36 | CD36 antigen (collagen | 6.38989E−06 | 0.039 | −4.35 | −1.93 | 2.85459 | 1.28375 | 0.00000 |
| | | type I receptor, | | | | | | | |
| | | thrombospondin | | | | | | | |
| | | receptor) | | | | | | | |
| 205644_s_at | SNRPG | small nuclear | 7.90765E−06 | 0.051 | 1.54 | 1.15 | −1.08154 | −1.11673 | 0.00009 |
| | | ribonucleoprotein | | | | | | | |
| | | polypeptide G | | | | | | | |
| 201301_s_at | ANXA4 | annexin A4 | 8.19608E−06 | 0.032 | −1.64 | −1.25 | 2.41708 | 1.30646 | 0.00000 |
| 218009_s_at | PRC1 | protein regulator of | 8.19792E−06 | 0.039 | 1.74 | 1.09 | −1.27211 | −1.20454 | 0.00000 |
| | | cytokinesis 1 | | | | | | | |
| 221505_at | ANP32E | acidic (leucine-rich) | 8.97891E−06 | 0.042 | 1.65 | 1.16 | −1.11840 | −1.22003 | 0.00023 |
| | | nuclear phosphoprotein | | | | | | | |
| | | 32 family, member E | | | | | | | |
| 208626_s_at | VAT1 | vesicle amine transport | 9.26872E−06 | 0.044 | −1.96 | −1.30 | 2.59029 | 1.28150 | 0.00000 |
| | | protein 1 homolog (T | | | | | | | |
| | | californica) | | | | | | | |
| 201193_at | IDH1 | isocitrate | 9.80795E−06 | 0.037 | −1.76 | −1.17 | 2.67335 | 1.22401 | 0.00000 |
| | | dehydrogenase 1 | | | | | | | |
| | | (NADP+), soluble | | | | | | | |
| 212224_at | ALDH1A1 | aldehyde | 1.8723E−05 | 0.034 | −4.56 | −2.25 | 3.03924 | 1.60442 | 0.00000 |
| | | dehydrogenase 1 | | | | | | | |
| | | family, member A1 | | | | | | | |
| 204026_s_at | ZWINT | ZW10 interactor | 1.97022E−05 | 0.037 | 1.78 | 1.08 | −1.20958 | −1.21967 | 0.00000 |
| 202671_s_at | PDXK | pyridoxal (pyridoxine, | 2.17167E−05 | 0.026 | −1.57 | −1.13 | 2.31702 | 1.30177 | 0.00000 |
| | | vitamin B6) kinase | | | | | | | |
| 211658_at | PRDX2 | peroxiredoxin 2 | 2.25368E−05 | 0.026 | 1.67 | −1.02 | −1.24254 | −1.05441 | 0.00167 |
| 202345_s_at | FABP5 | fatty acid binding | 4.28861E−05 | 0.026 | −1.48 | −1.57 | −1.04410 | 1.06487 | 0.10321 |
| | | protein 5 (psoriasis- | | | | | | | |
| | | associated) | | | | | | | |
| 202096_s_at | BZRP | benzodiazapine | 6.47932E−05 | 0.037 | −1.78 | −1.24 | 2.44819 | 1.29796 | 0.00000 |
| | | receptor (peripheral) | | | | | | | |
| 204890_s_at | LCK | lymphocyte-specific | 9.45284E−05 | 0.047 | 1.53 | 1.09 | −1.18753 | −1.13461 | 0.00003 |
| | | protein tyrosine kinase | | | | | | | |
| 204252_at | CDK2 | cyclin-dependent | 0.000102989 | 0.037 | 1.70 | 1.16 | −1.16492 | −1.20192 | 0.00001 |
| | | kinase 2 | | | | | | | |
| 209906_at | C3AR1 | complement component | 0.000132024 | 0.037 | −1.51 | 1.21 | 2.41148 | 1.24719 | 0.00025 |
| | | 3a receptor 1 | | | | | | | |
| 203305_at | F13A1 | coagulation factor XIII, | 0.000159995 | 0.050 | −3.34 | −1.35 | 4.01106 | 1.39191 | 0.00002 |
| | | A1 polypeptide | | | | | | | |
| 213241_at | PLXNC1 | plexin C1 | 0.000258071 | 0.051 | −1.85 | −1.26 | 2.82837 | 1.28688 | 0.00000 |
| 212807_s_at | SORT1 | sortilin 1 | 0.000314093 | 0.037 | −1.65 | −1.04 | 2.29584 | 1.21623 | 0.00011 |
| 204023_at | RFC4 | replication factor C | 0.000839626 | 0.039 | 2.01 | 1.33 | −1.27795 | −1.35643 | 0.00000 |
| | | (activator 1) 4, 37 kDa | | | | | | | |
| 212737_at | UNK_AL513583 | Consensus includes | 0.001029402 | 0.042 | −1.78 | −1.24 | 2.63324 | 1.22804 | 0.00000 |
| | | gb: AL513583 | | | | | | | |
| | | /FEA = EST | | | | | | | |
| | | /DB_XREF = gi: | | | | | | | |
| | | 12777077 | | | | | | | |
| | | /DB_XREF = est: | | | | | | | |
| | | AL513583 | | | | | | | |
| | | /CLONE = | | | | | | | |
| | | XCL0BA001ZA05 | | | | | | | |
| | | (3 prime) | | | | | | | |
| | | /UG = Hs.278242 | | | | | | | |
| | | tubulin, alpha, ubiquitous | | | | | | | |
| 217869_at | HSD17B12 | hydroxysteroid (17- | 0.001320365 | 0.034 | −1.54 | −1.13 | 2.16824 | 1.10397 | 0.00000 |
| | | beta) dehydrogenase | | | | | | | |
| | | 12 | | | | | | | |
| 208771_s_at | LTA4H | leukotriene A4 | 0.001377097 | 0.023 | −1.88 | −1.19 | 2.32896 | 1.27268 | 0.00000 |
| | | hydrolase | | | | | | | |
| 208146_s_at | CPVL | carboxypeptidase, | 0.001533097 | 0.044 | −2.13 | −1.16 | 3.00463 | 1.34877 | 0.00000 |
| | | vitellogenic-like | | | | | | | |
| 220147_s_at | C12ORF14 | chromosome 12 open | 0.001709512 | 0.039 | 1.67 | 1.21 | −1.23200 | −1.26285 | 0.00000 |
| | | reading frame 14 | | | | | | | |
| 209823_x_at | HLA-DQB1 | major histocompatibility | 0.001752874 | 0.037 | −1.62 | −1.03 | 2.39098 | 1.18216 | 0.00000 |
| | | complex, class II, DQ | | | | | | | |
| | | beta 1 | | | | | | | |
| 35820_at | GM2A | GM2 ganglioside | 0.002943026 | 0.039 | −2.07 | −1.25 | 2.79662 | 1.31813 | 0.00000 |
| | | activator protein | | | | | | | |
| 206545_at | CD28 | CD28 antigen (Tp44) | 0.003510526 | 0.050 | 1.74 | 1.09 | −1.15869 | −1.18821 | 0.00077 |
| 213274_s_at | UNK_AA020826 | Consensus includes | 0.004201615 | 0.043 | −2.38 | −1.55 | 2.97646 | 1.35275 | 0.00000 |
| | | gb: AA020826 | | | | | | | |
| | | /FEA = EST | | | | | | | |
| | | /DB_XREF = gi: | | | | | | | |
| | | 1484570 | | | | | | | |
| | | /DB_XREF = est: | | | | | | | |
| | | ze64b04.s1 | | | | | | | |
| | | /CLONE = IMAGE: | | | | | | | |
| | | 363727 | | | | | | | |
| | | /UG = Hs.297939 | | | | | | | |
| | | cathepsin B | | | | | | | |
| 207809_s_at | ATP6AP1 | ATPase, H+ | 0.004538564 | 0.047 | −1.66 | −1.11 | 2.57927 | 1.16448 | 0.00000 |
| | | transporting, lysosomal | | | | | | | |
| | | accessory protein 1 | | | | | | | |
| 203246_s_at | TUSC4 | tumor suppressor | 0.004645699 | 0.051 | 1.59 | −1.05 | −1.30864 | 1.05661 | 0.00088 |
| | | candidate 4 | | | | | | | |
| 201209_at | HDAC1 | histone deacetylase 1 | 0.006241482 | 0.033 | 1.64 | 1.09 | −1.14328 | −1.14707 | 0.00011 |
| 213762_x_at | RBMX | RNA binding motif | 0.008900231 | 0.039 | 1.53 | 1.19 | −1.10254 | −1.30752 | 0.00022 |
| | | protein, X-linked | | | | | | | |
| 203276_at | LMNB1 | lamin B1 | 0.009151755 | 0.039 | 2.08 | 1.22 | −1.13147 | −1.09517 | 0.02267 |
| 213734_at | RFC5 | replication factor C | 0.010142166 | 0.049 | −1.47 | −1.50 | 2.26061 | 1.22884 | 0.05227 |
| | | (activator 1) 5, 36.5 kDa | | | | | | | |
| 204362_at | SCAP2 | src family associated | 0.013347111 | 0.047 | −1.51 | −1.13 | 2.41775 | 1.22624 | 0.00000 |
| | | phosphoprotein 2 | | | | | | | |
| 206115_at | EGR3 | early growth response 3 | 0.018320525 | 0.040 | 1.25 | 1.59 | −1.07421 | −1.38983 | 0.62393 |
| 211189_x_at | CD84 | CD84 antigen | 0.018851741 | 0.049 | −1.66 | −1.07 | 2.34553 | 1.18502 | 0.00001 |
| | | (leukocyte antigen) | | | | | | | |
| 204867_at | GCHFR | GTP cyclohydrolase I | 0.018895749 | 0.049 | −1.65 | −1.03 | 2.20718 | 1.26803 | 0.01424 |
| | | feedback regulatory | | | | | | | |
| | | protein | | | | | | | |
| 211732_x_at | HNMT | histamine N- | 0.02881445 | 0.051 | −1.67 | −1.11 | 2.36589 | 1.25965 | 0.00002 |
| | | methyltransferase | | | | | | | |
| 39729_at | PRDX2 | peroxiredoxin 2 | 0.029677139 | 0.043 | 1.84 | 1.25 | −1.26039 | −1.31203 | 0.00000 |
| 204891_s_at | LCK | lymphocyte-specific | 0.045708277 | 0.039 | −1.68 | 1.05 | −1.24429 | −1.23424 | 0.00000 |
| | | protein tyrosine kinase | | | | | | | |
| 205382_s_at | DF | D component of | 0.046880329 | 0.050 | −3.75 | −2.16 | 3.14737 | 1.53959 | 0.00000 |
| | | complement (adipsin) | | | | | | | |
| 214765_s_at | ASAHL | N-acylsphingosine | 0.048876711 | 0.040 | −1.47 | −1.83 | 2.19068 | 1.55795 | 0.05899 |
| | | amidohydrolase (acid | | | | | | | |
| | | ceramidase)-like | | | | | | | |
| 200632_s_at | NDRG1 | N-myc downstream | 0.057430597 | 0.035 | −1.45 | −1.56 | 2.67072 | 1.30468 | 0.00000 |
| | | regulated gene 1 | | | | | | | |
| 213539_at | CD3D | CD3D antigen, delta | 0.064726579 | 0.037 | 1.71 | 1.10 | −1.26707 | −1.34377 | 0.00000 |
| | | polypeptide (TiT3 | | | | | | | |
| | | complex) | | | | | | | |
| 202107_s_at | MCM2 | MCM2 | 0.09483288 | 0.051 | 2.01 | 1.29 | −1.27544 | −1.29004 | 0.00000 |
| | | minichromosome | | | | | | | |
| | | maintenance deficient | | | | | | | |
| | | 2, mitotin (S. cerevisiae) | | | | | | | |
| 208713_at | E1B-AP5/ | E1B-55 kDa-associated | 0.098935737 | 0.037 | 1.59 | −1.03 | −1.06909 | 1.02425 | 0.16709 |
| | HNRPUL1 | protein 5 | | | | | | | |
| 56256_at | TAGLN | transgelin | 0.109489136 | 0.026 | −1.78 | −1.20 | 2.58208 | 1.23451 | 0.00000 |
| 208808_s_at | HMGB2 | high-mobility group | 0.129496408 | 0.042 | 1.77 | 1.19 | −1.12628 | −1.18281 | 0.00047 |
| | | box 2 | | | | | | | |
| 202801_at | PRKACA | protein kinase, cAMP- | 0.132972638 | 0.035 | −1.18 | −1.53 | 2.01979 | 1.26700 | 0.91560 |
| | | dependent, catalytic, | | | | | | | |
| | | alpha | | | | | | | |
| 201459_at | RUVBL2 | RuvB-like 2 (E. coli) | 0.13361792 | 0.051 | 2.05 | 1.33 | −1.17277 | −1.18809 | 0.00021 |
| 211668_s_at | PLAU | plasminogen activator, | 0.146042454 | 0.050 | −1.87 | −1.15 | 2.89709 | 1.39949 | 0.00000 |
| | | urokinase | | | | | | | |
| 200680_x_at | HMGB1 | high-mobility group | 0.148693618 | 0.039 | 1.53 | 1.15 | −1.08805 | −1.09443 | 0.01335 |
| | | box 1 | | | | | | | |
| 202887_s_at | DDIT4 | DNA-damage-inducible | 0.157499282 | 0.045 | 2.04 | 1.34 | −1.17104 | −1.18153 | 0.00017 |
| | | transcript 4 | | | | | | | |
| 210105_s_at | FYN | FYN oncogene related | 0.171850992 | 0.032 | 1.51 | 1.02 | −1.15741 | −1.15451 | 0.00004 |
| | | to SRC, FGR, YES | | | | | | | |
| 200931_s_at | VCL | vinculin | 0.246766588 | 0.047 | −1.51 | −1.13 | 2.02019 | 1.20026 | 0.01664 |
| 218561_s_at | C6ORF149 | chromosome 6 open | 0.304939358 | 0.037 | 1.52 | 1.06 | −1.18828 | −1.13299 | 0.00000 |
| | | reading frame 149 | | | | | | | |
| 213682_at | NUP50 | nucleoporin 50 kDa | 0.321069384 | 0.037 | 1.67 | 1.18 | −1.15465 | −1.16333 | 0.00041 |
| 200871_s_at | PSAP | prosaposin (variant | 0.322811966 | 0.044 | −1.73 | −1.25 | 2.51480 | 1.13582 | 0.00000 |
| | | Gaucher disease and | | | | | | | |
| | | variant metachromatic | | | | | | | |
| | | leukodystrophy) | | | | | | | |
| 213416_at | ITGA4 | integrin, alpha 4 | 0.329745187 | 0.051 | 1.66 | 1.07 | −1.20439 | −1.30097 | 0.00011 |
| | | (antigen CD49D, alpha | | | | | | | |
| | | 4 subunit of VLA-4 | | | | | | | |
| | | receptor) | | | | | | | |
| 205831_at | CD2 | CD2 antigen (p50), | 0.34485804 | 0.037 | 1.62 | 1.10 | −1.17336 | −1.24167 | 0.00001 |
| | | sheep red blood cell | | | | | | | |
| | | receptor | | | | | | | |
| 202858_at | U2AF1 | U2(RNU2) small | 0.345008521 | 0.046 | 1.72 | 1.17 | −1.19709 | −1.09997 | 0.00018 |
| | | nuclear RNA auxiliary | | | | | | | |
| | | factor 1 | | | | | | | |
| 201202_at | PCNA | proliferating cell nuclear | 0.345321173 | 0.037 | 1.73 | 1.03 | −1.20309 | −1.13777 | 0.00056 |
| | | antigen | | | | | | | |
| 201149_s_at | TIMP3 | tissue inhibitor of | 0.360488653 | 0.050 | −3.41 | −2.13 | 2.23363 | 1.01499 | 0.01495 |
| | | metalloproteinase 3 | | | | | | | |
| | | (Sorsby fundus | | | | | | | |
| | | dystrophy, | | | | | | | |
| | | pseudoinflammatory) | | | | | | | |
| 208795_s_at | MCM7 | MCM7 | 0.361405722 | 0.050 | 2.03 | 1.35 | −1.33200 | −1.28460 | 0.00000 |
| | | minichromosome | | | | | | | |
| | | maintenance deficient 7 | | | | | | | |
| | | (S. cerevisiae) | | | | | | | |
| 205961_s_at | UNK_NM_004682/ | gb: NM_004682.1 | 0.410418881 | 0.048 | 1.66 | 1.01 | −1.25230 | −1.11054 | 0.00058 |
| | PSIP1/ | /DEF = Homo sapiens | | | | | | | |
| | PSIP2 | PC4 and SFRS1 | | | | | | | |
| | | interacting protein 2 | | | | | | | |
| | | (PSIP2), mRNA. | | | | | | | |
| | | /FEA = mRNA | | | | | | | |
| | | /GEN = PSIP2 | | | | | | | |
| | | /PROD = PC4 and | | | | | | | |
| | | SFRS1 interacting | | | | | | | |
| | | protein 2 | | | | | | | |
| | | /DB_XREF = gi: | | | | | | | |
| | | 4758869 | | | | | | | |
| | | /UG = Hs.306179 PC4 | | | | | | | |
| | | and SFRS1 interacting | | | | | | | |
| | | protein 2 | | | | | | | |
| | | /FL = gb: AF098483.1 | | | | | | | |
| | | gb: NM_004682.1 | | | | | | | |
| 213170_at | GPX7 | glutathione peroxidase 7 | 0.421808045 | 0.039 | 1.53 | 1.06 | −1.19560 | −1.19838 | 0.00000 |
| 203554_x_at | PTTG1 | pituitary tumor- | 0.453785538 | 0.047 | 1.52 | 1.10 | −1.18803 | −1.11054 | 0.00000 |
| | | transforming 1 | | | | | | | |
| 215707_s_at | PRNP | prion protein (p27-30) | 0.46971613 | 0.026 | −1.55 | −1.02 | 2.22311 | 1.10475 | 0.00019 |
| | | (Creutzfeld-Jakob | | | | | | | |
| | | disease, Gerstmann- | | | | | | | |
| | | Strausler-Scheinker | | | | | | | |
| | | syndrome, fatal familial | | | | | | | |
| | | insomnia) | | | | | | | |
| 211951_at | NOLC1 | nucleolar and coiled- | 0.519086257 | 0.051 | 1.73 | 1.26 | −1.21682 | −1.20954 | 0.00000 |
| | | body phosphoprotein 1 | | | | | | | |
| 218039_at | NUSAP1 | nucleolar and spindle | 0.527835161 | 0.044 | 1.81 | 1.22 | −1.19697 | −1.15555 | 0.00000 |
| | | associated protein 1 | | | | | | | |
| 218308_at | TACC3 | transforming, acidic | 0.542167461 | 0.026 | 1.63 | 1.10 | −1.18516 | −1.02801 | 0.00030 |
| | | coiled-coil containing | | | | | | | |
| | | protein 3 | | | | | | | |
| 209606_at | PSCDBP | pleckstrin homology, | 0.554466438 | 0.041 | 1.58 | 1.01 | −1.20980 | −1.06716 | 0.00001 |
| | | Sec7 and coiled-coil | | | | | | | |
| | | domains, binding | | | | | | | |
| | | protein | | | | | | | |
| 200672_x_at | SPTBN1 | spectrin, beta, non- | 0.555737816 | 0.045 | 1.35 | 1.53 | −1.17899 | −1.47818 | 0.03013 |
| | | erythrocytic 1 | | | | | | | |
| 213073_at | ZFYVE26 | zinc finger, FYVE | 0.66856305 | 0.037 | −1.59 | −1.02 | 2.16653 | 1.10716 | 0.00027 |
| | | domain containing 26 | | | | | | | |
| 208956_x_at | DUT | dUTP pyrophosphatase | 0.690283883 | 0.051 | 1.77 | 1.25 | −1.15682 | −1.20873 | 0.00000 |
| 216237_s_at | MCM5 | MCM5 | 0.754327403 | 0.051 | 1.79 | 1.22 | −1.23227 | −1.22449 | 0.00000 |
| | | minichromosome | | | | | | | |
| | | maintenance deficient | | | | | | | |
| | | 5, cell division cycle 46 | | | | | | | |
| | | (S. cerevisiae) | | | | | | | |
| 219971_at | IL21R | interleukin 21 receptor | 0.772871673 | 0.047 | 1.55 | 1.07 | −1.11723 | −1.01764 | 0.00211 |
| 201305_x_at | UNK_AV712577 | Consensus includes | 0.816317838 | 0.051 | 1.62 | 1.11 | −1.02557 | −1.10495 | 0.37052 |
| | | gb: AV712577 | | | | | | | |
| | | /FEA = EST | | | | | | | |
| | | /DB_XREF = gi: | | | | | | | |
| | | 10731883 | | | | | | | |
| | | /DB_XREF = est: | | | | | | | |
| | | AV712577 | | | | | | | |
| | | /CLONE = DCAAUH03 | | | | | | | |
| | | /UG = Hs.84264 acidic | | | | | | | |
| | | protein rich in leucines | | | | | | | |
| | | /FL = gb: U70439.1 | | | | | | | |
| | | gb: NM_006401.1 | | | | | | | |
| 200956_s_at | SSRP1 | structure specific | 0.817518612 | 0.050 | 1.75 | 1.26 | −1.25697 | −1.26092 | 0.00001 |
| | | recognition protein 1 | | | | | | | |
| 218231_at | NAGK | N-acetylglucosamine | 0.87121261 | 0.051 | −1.54 | −1.09 | 2.75156 | 1.35002 | 0.00000 |
| | | kinase | | | | | | | |
| 221078_s_at | UNK_NM_018084 | gb: NM_018084.1 | 0.891607875 | 0.039 | −1.68 | −1.14 | −1.01365 | 1.00790 | 0.96171 |
| | | /DEF = Homo sapiens | | | | | | | |
| | | hypothetical protein | | | | | | | |
| | | FLJ10392 (FLJ10392), | | | | | | | |
| | | mRNA. /FEA = mRNA | | | | | | | |
| | | /GEN = FLJ10392 | | | | | | | |
| | | /PROD = hypothetical | | | | | | | |
| | | protein FLJ10392 | | | | | | | |
| | | /DB_XREF = gi: | | | | | | | |
| | | 8922402 | | | | | | | |
| | | /UG = Hs.20887 | | | | | | | |
| | | hypothetical protein | | | | | | | |
| | | FLJ10392 | | | | | | | |
| | | /FL = gb: NM_018084.1 | | | | | | | |
| 219282_s_at | UNK_NM_015930 | gb: NM_015930.1 | 0.903159358 | 0.039 | −1.66 | −1.21 | 2.17434 | 1.24082 | 0.00019 |
| | | /DEF = Homo sapiens | | | | | | | |
| | | vanilloid receptor-like | | | | | | | |
| | | protein 1 (VRL-1), | | | | | | | |
| | | mRNA. /FEA = mRNA | | | | | | | |
| | | /GEN = VRL-1 | | | | | | | |
| | | /PROD = vanilloid | | | | | | | |
| | | receptor-like protein 1 | | | | | | | |
| | | /DB_XREF = gi: | | | | | | | |
| | | 7706764 | | | | | | | |
| | | /UG = Hs.279746 | | | | | | | |
| | | vanilloid receptor-like | | | | | | | |
| | | protein 1 | | | | | | | |
| | | /FL = gb: AF129112.1 | | | | | | | |
| | | gb: NM_015930.1 | | | | | | | |
| 209765_at | ADAM19 | a disintegrin and | 0.932958423 | 0.047 | 2.16 | 1.44 | −1.20589 | −1.36141 | 0.00001 |
| | | metalloproteinase | | | | | | | |
| | | domain 19 (meltrin | | | | | | | |
| | | beta) | | | | | | | |
| 204347_at | AK3 | adenylate kinase 3 | Probeset did | 0.048 | −1.25 | −1.67 | 2.30519 | 1.31550 | 0.05215 |
| | | | not pass | | | | | | |
| | | | filters in | | | | | | |
| | | | PBMC | | | | | | |
| | | | analysis | | | | | | |
| 201971_s_at | ATP6V1A | ATPase, H+ | Probeset did | 0.044 | −1.52 | 1.02 | 2.44558 | 1.11698 | 0.00064 |
| | | transporting, lysosomal | not pass | | | | | | |
| | | 70 kDa, V1 subunit A | filters in | | | | | | |
| | | | PBMC | | | | | | |
| | | | analysis | | | | | | |
| 218264_at | BCCIP | BRCA2 and CDKN1A | Probeset did | 0.037 | 1.61 | −1.02 | −1.25287 | −1.12121 | 0.00010 |
| | | interacting protein | not pass | | | | | | |
| | | | filters in | | | | | | |
| | | | PBMC | | | | | | |
| | | | analysis | | | | | | |
| 218542_at | C10ORF3 | chromosome 10 open | Probeset did | 0.045 | 2.26 | 1.36 | −1.25517 | −1.33477 | 0.00006 |
| | | reading frame 3 | not pass | | | | | | |
| | | | filters in | | | | | | |
| | | | PBMC | | | | | | |
| | | | analysis | | | | | | |
| 203213_at | CDC2 | cell division cycle 2, G1 | Probeset did | 0.045 | 1.97 | 1.12 | −1.16295 | −1.25844 | 0.00435 |
| | | to S and G2 to M | not pass | | | | | | |
| | | | filters in | | | | | | |
| | | | PBMC | | | | | | |
| | | | analysis | | | | | | |
| 208168_s_at | CHIT1 | chitinase 1 | Probeset did | 0.044 | −3.59 | −3.01 | 2.80342 | 2.01259 | 0.00014 |
| | | (chitotriosidase) | not pass | | | | | | |
| | | | filters in | | | | | | |
| | | | PBMC | | | | | | |
| | | | analysis | | | | | | |
| 210757_x_at | DAB2 | disabled homolog 2, | Probeset did | 0.048 | −1.90 | −1.34 | 2.52393 | 1.32582 | 0.00000 |
| | | mitogen-responsive | not pass | | | | | | |
| | | phosphoprotein | filters in | | | | | | |
| | | (Drosophila) | PBMC | | | | | | |
| | | | analysis | | | | | | |
| 201279_s_at | DAB2 | disabled homolog 2, | Probeset did | 0.037 | −2.03 | −1.41 | 2.44170 | 1.41267 | 0.00000 |
| | | mitogen-responsive | not pass | | | | | | |
| | | phosphoprotein | filters in | | | | | | |
| | | (Drosophila) | PBMC | | | | | | |
| | | | analysis | | | | | | |
| 204015_s_at | DUSP4 | dual specificity | Probeset did | 0.039 | 2.70 | 1.43 | −1.34403 | −1.15736 | 0.00000 |
| | | phosphatase 4 | not pass | | | | | | |
| | | | filters in | | | | | | |
| | | | PBMC | | | | | | |
| | | | analysis | | | | | | |
| 204014_at | DUSP4 | dual specificity | Probeset did | 0.051 | 2.88 | 1.64 | −1.39272 | −1.38782 | 0.00000 |
| | | phosphatase 4 | not pass | | | | | | |
| | | | filters in | | | | | | |
| | | | PBMC | | | | | | |
| | | | analysis | | | | | | |
| 205738_s_at | FABP3 | fatty acid binding | Probeset did | 0.039 | −3.76 | −1.92 | 2.57387 | −1.03661 | 0.00150 |
| | | protein 3, muscle and | not pass | | | | | | |
| | | heart (mammary- | filters in | | | | | | |
| | | derived growth inhibitor) | PBMC | | | | | | |
| | | | analysis | | | | | | |
| 219990_at | FLJ23311 | FLJ23311 protein | Probeset did | 0.051 | 1.77 | 1.01 | −1.36156 | 1.04174 | 0.00001 |
| | | | not pass | | | | | | |
| | | | filters in | | | | | | |
| | | | PBMC | | | | | | |
| | | | analysis | | | | | | |
| 33646_g_at | GM2A | GM2 ganglioside | Probeset did | 0.039 | −2.26 | −1.09 | 2.49882 | 1.34398 | 0.00011 |
| | | activator protein | not pass | | | | | | |
| | | | filters in | | | | | | |
| | | | PBMC | | | | | | |
| | | | analysis | | | | | | |
| 209727_at | GM2A | GM2 ganglioside | Probeset did | 0.039 | −2.05 | −1.02 | 2.41500 | 1.21143 | 0.00111 |
| | | activator protein | not pass | | | | | | |
| | | | filters in | | | | | | |
| | | | PBMC | | | | | | |
| | | | analysis | | | | | | |
| 219697_at | HS3ST2 | heparan sulfate | Probeset did | 0.048 | −5.42 | −2.58 | 4.36282 | 1.28788 | 0.00000 |
| | | (glucosamine) 3-O- | not pass | | | | | | |
| | | sulfotransferase 2 | filters in | | | | | | |
| | | | PBMC | | | | | | |
| | | | analysis | | | | | | |
| 204059_s_at | ME1 | malic enzyme 1, | Probeset did | 0.037 | −2.16 | −1.35 | 2.98562 | 1.51828 | 0.00000 |
| | | NADP(+)-dependent, | not pass | | | | | | |
| | | cytosolic | filters in | | | | | | |
| | | | PBMC | | | | | | |
| | | | analysis | | | | | | |
| 204825_at | MELK | maternal embryonic | Probeset did | 0.037 | 1.71 | 1.08 | −1.22799 | −1.21344 | 0.00001 |
| | | leucine zipper kinase | not pass | | | | | | |
| | | | filters in | | | | | | |
| | | | PBMC | | | | | | |
| | | | analysis | | | | | | |
| 213599_at | OIP5 | Opa-interacting protein 5 | Probeset did | 0.044 | 1.60 | 1.05 | −1.14145 | −1.06702 | 0.00008 |
| | | | not pass | | | | | | |
| | | | filters in | | | | | | |
| | | | PBMC | | | | | | |
| | | | analysis | | | | | | |
| 203060_s_at | PAPSS2 | 3′-phosphoadenosine | Probeset did | 0.020 | −1.45 | −1.68 | 2.16243 | 1.12973 | 0.06718 |
| | | 5′-phosphosulfate | not pass | | | | | | |
| | | synthase 2 | filters in | | | | | | |
| | | | PBMC | | | | | | |
| | | | analysis | | | | | | |
| 201411_s_at | PLEKHB2 | pleckstrin homology | Probeset did | 0.039 | −1.67 | 1.06 | 2.51027 | 1.27660 | 0.00000 |
| | | domain containing, | not pass | | | | | | |
| | | family B (evectins) | filters in | | | | | | |
| | | member 2 | PBMC | | | | | | |
| | | | analysis | | | | | | |
| 213007_at | POLG | polymerase (DNA | Probeset did | 0.032 | 1.85 | 1.16 | −1.16324 | −1.33724 | 0.00002 |
| | | directed), gamma | not pass | | | | | | |
| | | | filters in | | | | | | |
| | | | PBMC | | | | | | |
| | | | analysis | | | | | | |
| 222077_s_at | RACGAP1 | Rac GTPase activating | Probeset did | 0.037 | 1.67 | 1.00 | −1.16707 | −1.10782 | 0.00008 |
| | | protein 1 | not pass | | | | | | |
| | | | filters in | | | | | | |
| | | | PBMC | | | | | | |
| | | | analysis | | | | | | |
| 201614_s_at | RUVBL1 | RuvB-like 1 (E. coli) | Probeset did | 0.037 | 2.11 | 1.30 | −1.21501 | −1.14397 | 0.00009 |
| | | | not pass | | | | | | |
| | | | filters in | | | | | | |
| | | | PBMC | | | | | | |
| | | | analysis | | | | | | |
| 213119_at | SLC36A1 | solute carrier family 36 | Probeset did | 0.037 | −1.90 | 1.01 | 2.38457 | 1.27918 | 0.00330 |
| | | (proton/amino acid | not pass | | | | | | |
| | | symporter), member 1 | filters in | | | | | | |
| | | | PBMC | | | | | | |
| | | | analysis | | | | | | |
| 214830_at | SLC38A6 | solute carrier family 38, | Probeset did | 0.039 | −2.05 | −1.30 | 2.90795 | 1.20640 | 0.00000 |
| | | member 6 | not pass | | | | | | |
| | | | filters in | | | | | | |
| | | | PBMC | | | | | | |
| | | | analysis | | | | | | |
| 212110_at | SLC39A14 | solute carrier family 39 | Probeset did | 0.048 | 2.09 | 1.49 | −1.32287 | −1.56821 | 0.00000 |
| | | (zinc transporter), | not pass | | | | | | |
| | | member 14 | filters in | | | | | | |
| | | | PBMC | | | | | | |
| | | | analysis | | | | | | |
| 203473_at | SLCO2B1 | solute carrier organic | Probeset did | 0.039 | −1.60 | −1.00 | 2.60940 | 1.23684 | 0.00000 |
| | | anion transporter family, | not pass | | | | | | |
| | | member 2B1 | filters in | | | | | | |
| | | | PBMC | | | | | | |
| | | | analysis | | | | | | |
| 203472_s_at | SLCO2B1 | solute carrier organic | Probeset did | 0.037 | −1.67 | 1.08 | 2.69767 | 1.21147 | 0.00001 |
| | | anion transporter family, | not pass | | | | | | |
| | | member 2B1 | filters in | | | | | | |
| | | | PBMC | | | | | | |
| | | | analysis | | | | | | |
| 204240_s_at | SMC2L1 | SMC2 structural | Probeset did | 0.050 | 1.66 | 1.18 | −1.24470 | −1.26958 | 0.00001 |
| | | maintenance of | not pass | | | | | | |
| | | chromosomes 2-like 1 | filters in | | | | | | |
| | | (yeast) | PBMC | | | | | | |
| | | | analysis | | | | | | |
| 219519_s_at | SN | sialoadhesin | Probeset did | 0.050 | −1.80 | 1.38 | 4.37807 | 1.61784 | 0.00000 |
| | | | not pass | | | | | | |
| | | | filters in | | | | | | |
| | | | PBMC | | | | | | |
| | | | analysis | | | | | | |
| 204033_at | TRIP13 | thyroid hormone | Probeset did | 0.041 | 1.97 | 1.32 | −1.35764 | −1.31677 | 0.00000 |
| | | receptor interactor 13 | not pass | | | | | | |
| | | | filters in | | | | | | |
| | | | PBMC | | | | | | |
| | | | analysis | | | | | | |
| 222036_s_at | UNK_AI859865 | Consensus includes | Probeset did | 0.051 | 1.85 | 1.23 | −1.20317 | −1.28973 | 0.00001 |
| | | gb: AI859865 / | not pass | | | | | | |
| | | FEA = EST | filters in | | | | | | |
| | | /DB_XREF = gi: | PBMC | | | | | | |
| | | 5513481 | analysis | | | | | | |
| | | /DB_XREF = est: | | | | | | | |
| | | wm21f03.x1 | | | | | | | |
| | | /CLONE = IMAGE: | | | | | | | |
| | | 2436605 | | | | | | | |
| | | /UG = Hs.154443 | | | | | | | |
| | | minichromosome | | | | | | | |
| | | maintenance deficient | | | | | | | |
| | | (S. cerevisiae) 4 | | | | | | | |
| 201890_at | UNK_BE966236 | Consensus includes | Probeset did | 0.039 | 1.78 | 1.13 | −1.16726 | −1.20239 | 0.00002 |
| | | gb: BE966236 | not pass | | | | | | |
| | | /FEA = EST | filters in | | | | | | |
| | | /DB_XREF = gi: | PBMC | | | | | | |
| | | 11771437 | analysis | | | | | | |
| | | /DB_XREF = est: | | | | | | | |
| | | 601660172R1 | | | | | | | |
| | | /CLONE = IMAGE: | | | | | | | |
| | | 3905920 | | | | | | | |
| | | /UG = Hs.75319 | | | | | | | |
| | | ribonucleotide | | | | | | | |
| | | reductase M2 | | | | | | | |
| | | polypeptide | | | | | | | |
| | | /FL = gb: NM_001034.1 | | | | | | | |

Table 7b. Allergen-specific changes occur in the PBMC of asthmatics compared to the PBMC of healthy volunteers. The cPLA2 inhibitor 4-{3-[1-benzhydryl-5-chloro-2-(2-{[(2,6-dimethylbenzyl)sulfonyl] amino}ethyl)-1H-indol-3-yl]propyl}benzoic acid alters the expression profile of genes asthma specific allergen-responsive genes. Fold changes are averaged from the individual asthmatic (AOS) and healthy volunteers (WHV) changes. Affymetrix identification numbers, gene names and descriptions along with the False Discovery Rate (FDR) are given. The fourth column provides the FDR for the significance of the association of the gene with asthma in PBMCs prior to culture (that is, untreated PBMCs). The FDR was calculated in Spotfire using the deltas (changes in expression of allergen vs. no allergen) for each of the treatment groups.
NT—no treatment.

TABLE 8A
EFFECTS OF CPLA2 INHIBITION ON BASELINE
GENE EXPRESSION IN AOS
Table 8a: Changes in expression levels in the asthmatic population
upon treatment with a cPLA2 inhibitor (4-{3-[1-benzhydryl-5-
chloro-2-(2-{[(2,6-dimethylbenzyl)sulfonyl]amino}ethyl)-
1H-indol-3-yl]propyl}benzoic acid) in the absence of allergen
(no AG). The Affymetrix ID, gene name, fold change
and FDR are provided.

| | | Fold Change | FDR cPLA2 |
|---|---|---|---|
| | | cPLA2 inhibitor | inhibitor vs. |
| AFFY ID | Pub_Name | vs no AG AOS | no AG AOS |
| 209235_at | UNK_AL031600 | 1.586345 | 0.001164 |
| 205119_s_at | FPR1 | 1.437622 | 1.35E−07 |
| 219159_s_at | SLAMF7 | 1.420858 | 2.64E−07 |
| 217203_at | UNK_U08626 | 1.362142 | 0.003006 |
| 206148_at | IL3RA | 1.335115 | 0.004567 |
| 206637_at | P2RY14 | 1.331248 | 0.000179 |
| 218345_at | HCA112 | 1.328444 | 1.06E−06 |
| 210146_x_at | LILRB2 | 1.318149 | 0.000949 |
| 205003_at | DOCK4 | 1.309745 | 6.85E−06 |
| 206631_at | PTGER2 | 1.306624 | 1.33E−05 |
| 202510_s_at | TNFAIP2 | 1.299963 | 3.60E−07 |
| 203922_s_at | CYBB | 1.297689 | 4.56E−05 |
| 201060_x_at | UNK_AI537887 | 1.29652 | 0.000319 |
| 202660_at | UNK_AA834576 | 1.29057 | 8.96E−05 |
| 218404_at | SNX10 | 1.280193 | 3.46E−06 |
| 202917_s_at | S100A8 | 1.272875 | 2.00E−05 |
| 204929_s_at | VAMP5 | 1.27273 | 4.04E−05 |
| 209267_s_at | SLC39A8 | 1.260972 | 2.81E−05 |
| 204881_s_at | UGCG | 1.260704 | 0.000176 |
| 221477_s_at | SOD2 | 1.258651 | 0.000377 |
| 202308_at | SREBF1 | 1.255364 | 0.002559 |
| 219869_s_at | SLC39A8 | 1.25433 | 2.54E−05 |
| 206453_s_at | NDRG2 | 1.243037 | 0.015054 |
| 219938_s_at | PSTPIP2 | 1.241964 | 0.000121 |
| 202087_s_at | CTSL | 1.240092 | 1.25E−06 |
| 221935_s_at | FLJ13078 | 1.2302 | 0.005815 |
| 220832_at | TLR8 | 1.226735 | 0.044699 |
| 202357_s_at | BF | 1.221206 | 0.006523 |
| 204759_at | CHC1L | 1.220398 | 0.009987 |
| 214590_s_at | UBE2D1 | 1.216818 | 0.005901 |
| 203973_s_at | CEBPD | 1.216104 | 0.000358 |
| 205992_s_at | IL15 | 1.215403 | 0.007144 |
| 219403_s_at | HPSE | 1.207669 | 0.021709 |
| 210305_at | PDE4DIP | 1.205939 | 0.008339 |
| 213017_at | UNK_AL534702 | 1.205447 | 0.005738 |
| 219316_s_at | C14ORF58 | 1.205201 | 0.000132 |
| 200986_at | SERPING1 | 1.204703 | 0.009086 |
| 214179_s_at | NFE2L1 | 1.203841 | 0.000979 |
| 217731_s_at | ITM2B | 1.203264 | 0.013912 |
| 218323_at | RHOT1 | 1.193619 | 0.001854 |
| 215111_s_at | TGFB1I4 | 1.193198 | 0.000255 |
| 211776_s_at | EPB41L3 | 1.192667 | 0.004677 |
| 205708_s_at | TRPM2 | 1.190746 | 0.020778 |
| 218983_at | C1RL | 1.190239 | 0.011201 |
| 211458_s_at | GABARAPL3 | 1.188806 | 0.03412 |
| 205770_at | GSR | 1.187953 | 0.021762 |
| 211795_s_at | FYB | 1.187179 | 0.002022 |
| 203853_s_at | GAB2 | 1.18636 | 0.049636 |
| 202284_s_at | CDKN1A | 1.185603 | 0.001132 |
| 210784_x_at | LILRB3 | 1.183796 | 0.007478 |
| 204961_s_at | NCF1 | 1.18374 | 0.001514 |
| 214058_at | MYCL1 | 1.178689 | 0.043656 |
| 208864_s_at | TXN | 1.178136 | 1.32E−05 |
| 208700_s_at | TKT | 1.176828 | 0.002725 |
| 217789_at | SNX6 | 1.175342 | 0.003081 |
| 218132_s_at | LENG5 | 1.174979 | 0.001351 |
| 217024_x_at | UNK_AC004832 | 1.173501 | 0.020905 |
| 201146_at | NFE2L2 | 1.172684 | 0.001963 |
| 212090_at | GRINA | 1.16814 | 0.001033 |
| 212681_at | EPB41L3 | 1.165553 | 0.037946 |
| 201118_at | PGD | 1.164569 | 0.001642 |
| 200759_x_at | NFE2L1 | 1.164558 | 0.003402 |
| 209028_s_at | ABI1 | 1.164247 | 0.013128 |
| 204049_s_at | UNK_NM_014721 | 1.163572 | 0.019982 |
| 206710_s_at | EPB41L3 | 1.162744 | 0.020984 |
| 219055_at | FLJ10379 | 1.159941 | 0.003603 |
| 218196_at | OSTM1 | 1.159304 | 0.002974 |
| 214733_s_at | UNK_AL031427 | 1.158731 | 0.012153 |
| 219806_s_at | FN5 | 1.158624 | 2.72E−05 |
| 219243_at | HIMAP4 | 1.157977 | 0.001322 |
| 201704_at | ENTPD6 | 1.155032 | 0.047661 |
| 214084_x_at | UNK_AW072388 | 1.153171 | 2.89E−05 |
| 204034_at | ETHE1 | 1.151614 | 2.56E−07 |
| 221765_at | UGCG | 1.150742 | 0.049492 |
| 216609_at | TXN | 1.149385 | 0.032642 |
| 204715_at | PANX1 | 1.14883 | 0.017576 |
| 203514_at | MAP3K3 | 1.14733 | 0.00065 |
| 204747_at | IFIT3 | 1.145197 | 0.016025 |
| 200629_at | WARS | 1.145082 | 0.00882 |
| 221485_at | B4GALT5 | 1.13993 | 0.003164 |
| 218549_s_at | CGI-90 | 1.138943 | 0.00406 |
| 208092_s_at | DKFZP566A1524 | 1.136332 | 0.017286 |
| 200070_at | C2ORF24 | 1.135368 | 0.021953 |
| 201943_s_at | CPD | 1.134729 | 0.003363 |
| 207627_s_at | TFCP2 | 1.134158 | 0.026909 |
| 205285_s_at | FYB | 1.133003 | 0.003045 |
| 203132_at | RB1 | 1.132512 | 0.027985 |
| 218924_s_at | CTBS | 1.131614 | 0.020996 |
| 211150_s_at | UNK_J03866 | 1.129014 | 0.049776 |
| 203595_s_at | IFIT5 | 1.126717 | 0.030992 |
| 203883_s_at | RAB11-FIP2 | 1.126264 | 0.028179 |
| 214257_s_at | SEC22L1 | 1.124313 | 0.04559 |
| 201940_at | CPD | 1.12078 | 0.043162 |
| 221744_at | HAN11 | 1.120298 | 0.004234 |
| 201160_s_at | CSDA | 1.120022 | 0.030516 |
| 204048_s_at | PHACTR2 | 1.118589 | 0.037171 |
| 211752_s_at | NDUFS7 | 1.117739 | 0.001951 |
| 211977_at | UNK_AK024651 | 1.117397 | 0.019171 |
| 221484_at | B4GALT5 | 1.117364 | 0.000669 |
| 212216_at | KIAA0436 | 1.116793 | 0.00718 |
| 203350_at | AP1G1 | 1.116666 | 0.047036 |
| 201132_at | HNRPH2 | 1.115468 | 0.003503 |
| 202538_s_at | DKFZP564O123 | 1.115271 | 0.004896 |
| 212634_at | UNK_AW298092 | 1.115201 | 0.018555 |
| 205170_at | STAT2 | 1.113818 | 0.043074 |
| 203481_at | C10ORF6 | 1.113343 | 0.040084 |
| 207571_x_at | C1ORF38 | 1.113002 | 6.05E−05 |
| 208745_at | ATP5L | 1.112287 | 0.028784 |
| 210136_at | MBP | 1.112036 | 0.018185 |
| 212051_at | WIRE | 1.109846 | 0.050772 |
| 206491_s_at | NAPA | 1.107334 | 0.008129 |
| 222209_s_at | FLJ22104 | 1.105786 | 0.021397 |
| 214470_at | KLRB1 | 1.10498 | 0.039239 |
| 202073_at | UNK_AV757675 | 1.104795 | 0.038592 |
| 221002_s_at | DC-TM4F2 | 1.104109 | 0.012613 |
| 200800_s_at | HSPA1A | 1.10336 | 0.018101 |
| 212255_s_at | ATP2C1 | 1.103152 | 0.034348 |
| 201463_s_at | TALDO1 | 1.102454 | 1.91E−06 |
| 201063_at | RCN1 | 1.101474 | 0.016187 |
| 200628_s_at | WARS | 1.101087 | 0.040796 |
| 209155_s_at | NT5C2 | 1.10023 | 0.024246 |
| 209417_s_at | IFI35 | 1.099393 | 0.008611 |
| 210768_x_at | LOC54499 | 1.098836 | 0.031418 |
| 202536_at | DKFZP564O123 | 1.096731 | 0.045595 |
| 211475_s_at | BAG1 | 1.096164 | 0.003453 |
| 209814_at | ZNF330 | 1.095233 | 0.01521 |
| 213077_at | YTHDC2 | 1.0942 | 0.037152 |
| 221751_at | PANK3 | 1.091237 | 0.027315 |
| 201136_at | PLP2 | 1.090913 | 0.011343 |
| 217941_s_at | ERBB2IP | 1.09084 | 0.038268 |
| 64064_at | UNK_AI435089 | 1.090179 | 0.001751 |
| 218583_s_at | RP42 | 1.088949 | 0.003808 |
| 201260_s_at | SYPL | 1.088316 | 0.032932 |
| 218388_at | PGLS | 1.087198 | 0.039717 |
| 200616_s_at | KIAA0152 | 1.086841 | 0.050706 |
| 212796_s_at | KIAA1055 | 1.086506 | 0.020244 |
| 201762_s_at | PSME2 | 1.08581 | 0.000219 |
| 221492_s_at | APG3L | 1.084439 | 0.009268 |
| 212268_at | SERPINB1 | 1.083094 | 0.027242 |
| 203745_at | HCCS | 1.082342 | 0.005607 |
| 200868_s_at | ZNF313 | 1.081647 | 0.021934 |
| 209063_x_at | UNK_BF248165 | 1.081591 | 0.045324 |
| 209479_at | C6ORF80 | 1.081092 | 0.016146 |
| 207121_s_at | MAPK6 | 1.075755 | 0.030433 |
| 212202_s_at | DKFZP564G2022 | 1.075118 | 0.013556 |
| 202266_at | TTRAP | 1.074272 | 0.002134 |
| 201649_at | UBE2L6 | 1.073528 | 0.006961 |
| 209969_s_at | STAT1 | 1.073128 | 0.029574 |
| 201734_at | CLCN3 | 1.07085 | 0.002958 |
| 200615_s_at | AP2B1 | 1.067719 | 0.044093 |
| 200887_s_at | STAT1 | 1.067568 | 0.042978 |
| 217823_s_at | UBE2J1 | 1.067084 | 0.028179 |
| 220741_s_at | PPA2 | 1.065864 | 0.019088 |
| 200085_s_at | TCEB2 | 1.06158 | 0.043887 |
| 200653_s_at | CALM1 | 1.061499 | 0.025794 |
| 200794_x_at | DAZAP2 | 1.0582 | 0.011776 |
| 204246_s_at | DCTN3 | 1.0568 | 0.034439 |
| 201068_s_at | PSMC2 | 1.053276 | 0.048613 |
| 208742_s_at | SAP18 | 1.051136 | 0.012658 |
| 209248_at | GHITM | 1.050156 | 0.050459 |
| 208909_at | UQCRES1 | −1.04699 | 0.037486 |
| 222021_x_at | UNK_AI348006 | −1.04748 | 0.011927 |
| 201049_s_at | RPS18 | −1.04837 | 0.029081 |
| 211378_x_at | UNK_BC001224 | −1.05156 | 0.048769 |
| 213414_s_at | RPS19 | −1.05343 | 0.028365 |
| 208799_at | UNK_BC004146 | −1.05377 | 0.042248 |
| 203090_at | SDF2 | −1.05515 | 0.047912 |
| 201371_s_at | CUL3 | −1.05736 | 0.026128 |
| 221488_s_at | C6ORF82 | −1.05887 | 0.024801 |
| 212337_at | FLJ20618 | −1.05953 | 0.047349 |
| 216250_s_at | UNK_X77598 | −1.0634 | 0.005887 |
| 221476_s_at | RPL15 | −1.06561 | 0.000772 |
| 200857_s_at | NCOR1 | −1.06574 | 0.032987 |
| 200609_s_at | WDR1 | −1.0659 | 0.012107 |
| 209685_s_at | PRKCB1 | −1.0669 | 0.0041 |
| 203545_at | ALG8 | −1.06839 | 0.016431 |
| 208842_s_at | GORASP2 | −1.06902 | 0.028331 |
| 217939_s_at | AFTIPHILIN | −1.0693 | 0.028209 |
| 217871_s_at | MIF | −1.07068 | 0.049402 |
| 202135_s_at | ACTR1B | −1.07478 | 0.026695 |
| 210676_x_at | RANBP2L1 | −1.07568 | 0.033332 |
| 209827_s_at | IL16 | −1.07572 | 0.010619 |
| 209429_x_at | EIF2B4 | −1.07661 | 0.01249 |
| 213295_at | CYLD | −1.07723 | 0.015718 |
| 218681_s_at | SDF2L1 | −1.07733 | 0.032152 |
| 204060_s_at | PRKX | −1.07766 | 0.039211 |
| 202771_at | FAM38A | −1.07926 | 0.031054 |
| 213065_at | MGC23401 | −1.07931 | 0.041609 |
| 209444_at | RAP1GDS1 | −1.08044 | 0.036512 |
| 219133_at | FLJ20604 | −1.08056 | 0.042091 |
| 215493_x_at | UNK_AL121936 | −1.08091 | 0.032217 |
| 210646_x_at | RPL13A | −1.08149 | 0.010124 |
| 206968_s_at | NFRKB | −1.08243 | 0.037562 |
| 201678_s_at | DC12 | −1.0829 | 0.024433 |
| 221253_s_at | TXNDC5 | −1.08343 | 0.018168 |
| 222099_s_at | C19ORF13 | −1.08344 | 0.032097 |
| 206245_s_at | IVNS1ABP | −1.08475 | 0.045596 |
| 215031_x_at | RNF126 | −1.08611 | 0.037576 |
| 219678_x_at | DCLRE1C | −1.08677 | 0.04831 |
| 203012_x_at | RPL23A | −1.08838 | 0.04609 |
| 221011_s_at | LBH | −1.08859 | 0.024931 |
| 34858_at | KCTD2 | −1.08889 | 0.048227 |
| 218229_s_at | POGK | −1.08902 | 0.027197 |
| 222216_s_at | MRPL17 | −1.0896 | 0.009206 |
| 212144_at | UNK_AL021707 | −1.08973 | 0.016519 |
| 218617_at | TRIT1 | −1.09124 | 0.020429 |
| 219228_at | ZNF331 | −1.09152 | 0.030583 |
| 217168_s_at | HERPUD1 | −1.09166 | 0.019962 |
| 212987_at | UNK_AL031178 | −1.09201 | 0.001959 |
| 213649_at | UNK_AA524053 | −1.0924 | 0.010183 |
| 201686_x_at | API5 | −1.09254 | 0.041385 |
| 213689_x_at | RPL5 | −1.09337 | 0.002718 |
| 212827_at | IGHM | −1.09402 | 0.002764 |
| 211938_at | EIF4B | −1.09683 | 0.005007 |
| 218422_s_at | C13ORF10 | −1.09748 | 0.049603 |
| 201183_s_at | CHD4 | −1.09767 | 0.015111 |
| 218829_s_at | UNK_NM_017780 | −1.09778 | 0.04125 |
| 219122_s_at | ICF45 | −1.09808 | 0.050459 |
| 211144_x_at | TRG@ | −1.09881 | 0.022406 |
| 212118_at | RFP | −1.10087 | 0.041507 |
| 211948_x_at | XTP2 | −1.102 | 0.035509 |
| 218973_at | EFTUD1 | −1.10344 | 0.005679 |
| 210627_s_at | GCS1 | −1.10414 | 0.045098 |
| 220956_s_at | EGLN2 | −1.10503 | 0.011708 |
| 204116_at | IL2RG | −1.10607 | 0.014529 |
| 220934_s_at | UNK_NM_024084 | −1.10767 | 0.019768 |
| 202860_at | UNK_NM_014856 | −1.10793 | 0.046632 |
| 215806_x_at | TRGC2 | −1.10918 | 0.025161 |
| 218434_s_at | AACS | −1.10934 | 0.026471 |
| 206845_s_at | RNF40 | −1.10945 | 0.018576 |
| 200932_s_at | DCTN2 | −1.10945 | 0.020429 |
| 216044_x_at | UNK_AK027146 | −1.10998 | 0.018397 |
| 206042_x_at | SNURF | −1.11021 | 0.015617 |
| 218421_at | CERK | −1.11146 | 0.011131 |
| 201611_s_at | ICMT | −1.11198 | 0.041263 |
| 204735_at | PDE4A | −1.11225 | 0.003894 |
| 212001_at | SFRS14 | −1.11254 | 0.013306 |
| 213129_s_at | UNK_AI970157 | −1.11472 | 0.035588 |
| 208184_s_at | TMEM1 | −1.11502 | 0.013359 |
| 207268_x_at | ABI2 | −1.11584 | 0.048989 |
| 217903_at | STRN4 | −1.1194 | 0.049402 |
| 218153_at | FLJ12118 | −1.12084 | 0.030975 |
| 203363_s_at | KIAA0652 | −1.12112 | 0.00876 |
| 200710_at | ACADVL | −1.12119 | 0.018576 |
| 221918_at | UNK_AI742210 | −1.12142 | 0.03757 |
| 212710_at | CAMSAP1 | −1.12262 | 0.049424 |
| 215179_x_at | PGF | −1.12325 | 0.049802 |
| 203093_s_at | TIMM44 | −1.12368 | 0.019608 |
| 205238_at | FLJ12687 | −1.12408 | 0.050706 |
| 219551_at | EAF2 | −1.12452 | 0.043219 |
| 209014_at | MAGED1 | −1.12453 | 0.00055 |
| 214931_s_at | UNK_AC005070 | −1.1247 | 0.040432 |
| 213835_x_at | UNK_AL524262 | −1.12652 | 0.045098 |
| 207667_s_at | MAP2K3 | −1.12836 | 0.000641 |
| 203600_s_at | C4ORF8 | −1.13088 | 0.001408 |
| 218219_s_at | LANCL2 | −1.13109 | 0.037048 |
| 203580_s_at | UNK_NM_003983 | −1.13239 | 0.006961 |
| 209199_s_at | MEF2C | −1.13298 | 0.035269 |
| 217480_x_at | IGKV1OR15-118 | −1.13333 | 0.023686 |
| 218966_at | MYO5C | −1.13395 | 0.036778 |
| 209324_s_at | RGS16 | −1.13424 | 0.002336 |
| 213645_at | UNK_AF305057 | −1.13526 | 0.045098 |
| 209813_x_at | TRGV9 | −1.13544 | 0.007568 |
| 216207_x_at | IGKV1D-13 | −1.13574 | 0.046931 |
| 212232_at | FNBP4 | −1.13676 | 0.004885 |
| 211996_s_at | UNK_BG256504 | −1.13738 | 0.022959 |
| 209320_at | ADCY3 | −1.13778 | 0.013189 |
| 212572_at | UNK_AW779556 | −1.13834 | 0.008943 |
| 214496_x_at | MYST4 | −1.13856 | 0.015423 |
| 204651_at | NRF1 | −1.1398 | 0.048198 |
| 213133_s_at | GCSH | −1.14132 | 0.031896 |
| 202734_at | TRIP10 | −1.14167 | 0.013504 |
| 203914_x_at | HPGD | −1.1429 | 0.016495 |
| 211707_s_at | IQCB1 | −1.1434 | 0.027234 |
| 203524_s_at | MPST | −1.14418 | 0.014338 |
| 221820_s_at | MYST1 | −1.14419 | 0.009347 |
| 217418_x_at | MS4A1 | −1.14553 | 0.004452 |
| 210622_x_at | CDK10 | −1.14692 | 0.00694 |
| 221671_x_at | IGKC | −1.14731 | 0.003432 |
| 214118_x_at | PCM1 | −1.14818 | 0.041766 |
| 213615_at | C3F | −1.14918 | 0.045532 |
| 211576_s_at | SLC19A1 | −1.1495 | 0.014085 |
| 207339_s_at | LTB | −1.1498 | 5.44E−05 |
| 212176_at | UNK_AA902326 | −1.14997 | 0.009086 |
| 209007_s_at | NPD014 | −1.15008 | 0.018277 |
| 217189_s_at | UNK_AL137800 | −1.15041 | 0.019053 |
| 202109_at | ARFIP2 | −1.15065 | 0.004979 |
| 205441_at | FLJ22709 | −1.15167 | 0.013912 |
| 201876_at | PON2 | −1.15294 | 0.014077 |
| 203685_at | BCL2 | −1.15477 | 0.000473 |
| 206053_at | UNK_NM_014930 | −1.15477 | 0.018678 |
| 219123_at | ZNF232 | −1.15552 | 0.004285 |
| 209556_at | NCDN | −1.15556 | 0.045539 |
| 222108_at | UNK_AC004010 | −1.15582 | 0.002975 |
| 34031_i_at | CCM1 | −1.15954 | 0.020783 |
| 218064_s_at | AKAP8L | −1.15979 | 0.001919 |
| 222311_s_at | SFRS15 | −1.16041 | 0.043833 |
| 214836_x_at | UNK_BG536224 | −1.16162 | 0.032379 |
| 213650_at | GOLGIN-67 | −1.16203 | 0.049948 |
| 211548_s_at | HPGD | −1.16298 | 0.014263 |
| 210349_at | CAMK4 | −1.16416 | 0.037661 |
| 217892_s_at | EPLIN | −1.1643 | 7.87E−05 |
| 205297_s_at | CD79B | −1.16541 | 0.021955 |
| 218365_s_at | FLJ10514 | −1.16575 | 0.003806 |
| 214916_x_at | UNK_BG340548 | −1.16604 | 0.007683 |
| 201313_at | ENO2 | −1.1663 | 0.002356 |
| 204978_at | SFRS16 | −1.16684 | 0.044773 |
| 59433_at | UNK_N32185 | −1.16758 | 0.019809 |
| 211569_s_at | HADHSC | −1.1676 | 0.013161 |
| 218951_s_at | FLJ11323 | −1.16775 | 0.028487 |
| 221651_x_at | UNK_BC005332 | −1.16807 | 0.000277 |
| 219635_at | ZNF606 | −1.169 | 0.041776 |
| 210830_s_at | PON2 | −1.16916 | 0.036512 |
| 216594_x_at | AKR1C1 | −1.17116 | 0.006591 |
| 218914_at | CGI-41 | −1.17135 | 0.050248 |
| 212177_at | C6ORF111 | −1.17242 | 0.033258 |
| 201695_s_at | NP | −1.17345 | 0.001115 |
| 205804_s_at | T3JAM | −1.17886 | 0.01616 |
| 207315_at | CD226 | −1.17943 | 0.023998 |
| 218532_s_at | FLJ20152 | −1.18038 | 0.004822 |
| 219667_s_at | BANK1 | −1.18156 | 0.001287 |
| 206486_at | LAG3 | −1.18286 | 0.02257 |
| 217767_at | C3 | −1.18774 | 0.000775 |
| 214146_s_at | PPBP | −1.18803 | 0.040279 |
| 202149_at | UNK_AL136139 | −1.1911 | 0.004677 |
| 221219_s_at | KLHDC4 | −1.19191 | 0.016592 |
| 220059_at | BRDG1 | −1.19224 | 0.005132 |
| 204341_at | TRIM16 | −1.19422 | 0.037486 |
| 206105_at | FMR2 | −1.19425 | 0.020838 |
| 204899_s_at | UNK_BF247098 | −1.19642 | 0.009387 |
| 222041_at | UNK_BG235929 | −1.19733 | 0.014632 |
| 209995_s_at | TCL1A | −1.19738 | 9.87E−06 |
| 211643_x_at | UNK_L14457 | −1.19829 | 0.029203 |
| 205671_s_at | HLA-DOB | −1.19968 | 0.039059 |
| 213333_at | MDH2 | −1.19998 | 1.64E−05 |
| 207971_s_at | KIAA0582 | −1.20243 | 0.045282 |
| 214669_x_at | UNK_BG485135 | −1.205 | 0.013013 |
| 208591_s_at | PDE3B | −1.2054 | 0.003972 |
| 203878_s_at | MMP11 | −1.20771 | 0.035082 |
| 205718_at | ITGB7 | −1.20809 | 0.000172 |
| 214768_x_at | UNK_BG540628 | −1.20859 | 0.046608 |
| 210511_s_at | INHBA | −1.2099 | 0.037712 |
| 211245_x_at | KIR2DL4 | −1.21147 | 0.002296 |
| 214482_at | ZNF46 | −1.2161 | 0.009295 |
| 203759_at | SIAT4C | −1.21624 | 0.037589 |
| 219977_at | AIPL1 | −1.21715 | 0.023723 |
| 215946_x_at | UNK_AL022324 | −1.21824 | 0.004959 |
| 39318_at | TCL1A | −1.21933 | 4.95E−05 |
| 208490_x_at | HIST1H2BF | −1.21946 | 0.008047 |
| 212190_at | SERPINE2 | −1.22109 | 0.000365 |
| 217179_x_at | UNK_X79782 | −1.22119 | 0.017 |
| 208614_s_at | FLNB | −1.22448 | 0.018632 |
| 213474_at | KCTD7 | −1.2298 | 0.038808 |
| 219966_x_at | BANP | −1.23393 | 0.004185 |
| 209138_x_at | IGLC2 | −1.23399 | 0.002064 |
| 211635_x_at | UNK_M24670 | −1.23543 | 0.006375 |
| 205192_at | MAP3K14 | −1.24096 | 0.001892 |
| 204409_s_at | EIF1AY | −1.2419 | 0.049521 |
| 209031_at | IGSF4 | −1.24767 | 0.005491 |
| 209930_s_at | NFE2 | −1.25606 | 0.021289 |
| 216491_x_at | UNK_U80139 | −1.25612 | 0.041073 |
| 201718_s_at | EPB41L2 | −1.25705 | 0.004323 |
| 211881_x_at | IGLJ3 | −1.26026 | 0.009821 |
| 217239_x_at | UNK_AF044592 | −1.26225 | 0.00764 |
| 209374_s_at | IGHM | −1.26448 | 0.002961 |
| 205237_at | FCN1 | −1.26582 | 0.003884 |
| 205345_at | BARD1 | −1.26881 | 0.03388 |
| 211645_x_at | UNK_M85256 | −1.27036 | 0.005427 |
| 205001_s_at | DDX3Y | −1.27178 | 0.006716 |
| 205313_at | TCF2 | −1.28241 | 0.003275 |
| 221517_s_at | CRSP6 | −1.28397 | 0.000862 |
| 217996_at | PHLDA1 | −1.28458 | 4.95E−05 |
| 215176_x_at | UNK_AW404894 | −1.28566 | 0.00212 |
| 211637_x_at | UNK_L23516 | −1.28844 | 0.006434 |
| 218921_at | SIGIRR | −1.29187 | 0.002879 |
| 212592_at | IGJ | −1.29288 | 0.001652 |
| 215214_at | UNK_H53689 | −1.2952 | 0.018947 |
| 217997_at | PHLDA1 | −1.29553 | 5.43E−05 |
| 201109_s_at | THBS1 | −1.30257 | 0.050942 |
| 217236_x_at | UNK_S74639 | −1.30628 | 0.000545 |
| 208806_at | CHD3 | −1.30689 | 0.003023 |
| 201396_s_at | SGTA | −1.31072 | 0.003774 |
| 216984_x_at | IGLJ3 | −1.32536 | 0.031052 |
| 203946_s_at | ARG2 | −1.32844 | 1.85E−05 |
| 215949_x_at | UNK_BF002659 | −1.32881 | 0.024576 |
| 201158_at | NMT1 | −1.34115 | 0.029574 |
| 212259_s_at | PBXIP1 | −1.34246 | 0.01426 |
| 215701_at | UNK_AL109666 | −1.35384 | 0.005793 |
| 203887_s_at | THBD | −1.3739 | 0.001119 |
| 217378_x_at | IGKV1OR2-108 | −1.4079 | 0.000552 |
| 216401_x_at | UNK_AJ408433 | −1.46709 | 0.003302 |
| 205403_at | IL1R2 | −1.48361 | 0.000264 |
| 221286_s_at | PACAP | −1.51195 | 0.007556 |
| 206942_s_at | PMCH | −1.58783 | 1.65E−05 |

TABLE 8B
EFFECTS OF CPLA2 INHIBITION ON BASELINE
GENE EXPRESSION IN HV
Table 8b: Changes in expression levels in the healthy population
upon treatment with a cPLA2 inhibitor (4-{3-[1-benzhydryl-5-
chloro-2-(2-{[(2,6-dimethylbenzyl)sulfonyl]amino}ethyl)-
1H-indol-3-yl]propyl}benzoic acid) in the absence of allergen
(no AG). The Affymetrix ID, gene name, fold change
and FDR are provided.

| | | Fold Change | FDR cPLA2 |
|---|---|---|---|
| | | cPLA2 inhibitor | inhibitor vs. no |
| AFFY ID | Pub_Name | vs. no AG HV | AG HV |
| 211719_x_at | FN1 | −18.8559 | 0.014068 |
| 212464_s_at | FN1 | −16.6219 | 0.011477 |
| 210495_x_at | FN1 | −16.2745 | 0.0062 |
| 216442_x_at | FN1 | −15.6848 | 0.00701 |
| 201785_at | RNASE1 | −3.60232 | 0.029489 |
| 201147_s_at | TIMP3 | −3.46904 | 0.018928 |
| 219434_at | TREM1 | −3.32781 | 0.001808 |
| 207016_s_at | ALDH1A2 | −2.96189 | 0.010634 |
| 204580_at | MMP12 | −2.62073 | 0.041222 |
| 204468_s_at | TIE | −2.54569 | 0.028419 |
| 203980_at | FABP4 | −2.41561 | 0.012523 |
| 203915_at | CXCL9 | −2.37126 | 0.028181 |
| 205890_s_at | UBD | −2.24285 | 0.005399 |
| 201148_s_at | TIMP3 | −2.23249 | 0.017657 |
| 214770_at | MSR1 | −2.18514 | 0.036592 |
| 201149_s_at | TIMP3 | −2.14278 | 0.003571 |
| 219232_s_at | EGLN3 | −1.99244 | 0.010146 |
| 211887_x_at | MSR1 | −1.97619 | 0.025722 |
| 207900_at | CCL17 | −1.92303 | 0.028961 |
| 201951_at | ALCAM | −1.8264 | 0.034635 |
| 219024_at | PLEKHA1 | −1.79475 | 0.035257 |
| 204363_at | F3 | −1.76763 | 0.026021 |
| 205674_x_at | FXYD2 | −1.76609 | 0.024493 |
| 209122_at | ADFP | −1.72613 | 0.010954 |
| 210889_s_at | FCGR2B | −1.71682 | 0.034056 |
| 201666_at | TIMP1 | −1.69161 | 0.022468 |
| 218498_s_at | ERO1L | −1.67444 | 0.010146 |
| 207826_s_at | ID3 | −1.6685 | 0.046981 |
| 221748_s_at | TNS | −1.64643 | 0.038959 |
| 213164_at | MRPS6 | −1.64611 | 0.035257 |
| 212944_at | MRPS6 | −1.6163 | 0.048612 |
| 204655_at | CCL5 | −1.59955 | 0.037424 |
| 208423_s_at | MSR1 | −1.57337 | 0.036592 |
| 206978_at | CCR2 | −1.56547 | 0.025722 |
| 202345_s_at | FABP5 | −1.54723 | 0.001736 |
| 210830_s_at | PON2 | −1.54265 | 0.010146 |
| 202481_at | DHRS3 | −1.53615 | 0.044086 |
| 203789_s_at | SEMA3C | −1.53508 | 0.036563 |
| 204526_s_at | TBC1D8 | −1.52675 | 0.047362 |
| 217996_at | PHLDA1 | −1.5192 | 0.010954 |
| 202973_x_at | FAM13A1 | −1.51445 | 0.047434 |
| 217047_s_at | FAM13A1 | −1.51171 | 0.014068 |
| 203066_at | GALNAC4S-6ST | −1.49037 | 0.036563 |
| 211962_s_at | UNK_BG250310 | −1.48969 | 0.033126 |
| 34210_at | CDW52 | −1.48317 | 0.043438 |
| 212522_at | PDE8A | −1.47763 | 0.012641 |
| 217963_s_at | NGFRAP1 | −1.46766 | 0.028961 |
| 213167_s_at | UNK_BF982927 | −1.46724 | 0.02495 |
| 204472_at | GEM | −1.45864 | 0.028961 |
| 200885_at | MGC19531 | −1.45809 | 0.029489 |
| 204661_at | CDW52 | −1.45175 | 0.042269 |
| 203060_s_at | PAPSS2 | −1.45111 | 0.014068 |
| 202746_at | ITM2A | −1.44708 | 0.010543 |
| 209841_s_at | LRRN3 | −1.42413 | 0.036563 |
| 212239_at | UNK_AI680192 | −1.3785 | 0.033126 |
| 209147_s_at | PPAP2A | −1.37743 | 0.036563 |
| 200921_s_at | BTG1 | −1.3765 | 0.017817 |
| 201194_at | SEPW1 | −1.37233 | 0.00547 |
| 205685_at | CD86 | −1.3629 | 0.025722 |
| 218536_at | MRS2L | −1.36151 | 0.029771 |
| 208488_s_at | CR1 | −1.34805 | 0.034056 |
| 219326_s_at | B3GNT1 | −1.34266 | 0.036592 |
| 212828_at | SYNJ2 | −1.33969 | 0.032104 |
| 212179_at | C6ORF111 | −1.31823 | 0.036563 |
| 213093_at | PRKCA | −1.31683 | 0.025298 |
| 222108_at | UNK_AC004010 | −1.30522 | 0.040434 |
| 201719_s_at | EPB41L2 | −1.30361 | 0.00449 |
| 209813_x_at | TRGV9 | −1.29709 | 0.020082 |
| 222062_at | IL27RA | −1.29694 | 0.026121 |
| 200953_s_at | CCND2 | −1.28873 | 0.036563 |
| 60471_at | RIN3 | −1.27872 | 0.028419 |
| 202720_at | TES | −1.27071 | 0.047487 |
| 207339_s_at | LTB | −1.25874 | 0.035257 |
| 201760_s_at | WSB2 | −1.25757 | 0.015163 |
| 212375_at | EP400 | −1.25396 | 0.010146 |
| 203537_at | PRPSAP2 | −1.25358 | 0.032104 |
| 201565_s_at | ID2 | −1.2305 | 0.047362 |
| 208073_x_at | TTC3 | −1.22837 | 0.020082 |
| 212474_at | KIAA0241 | −1.21921 | 0.036563 |
| 222216_s_at | MRPL17 | −1.21005 | 0.014068 |
| 203087_s_at | KIF2 | −1.20274 | 0.044086 |
| 207668_x_at | TXNDC7 | −1.19975 | 0.008794 |
| 201778_s_at | KIAA0494 | −1.19393 | 0.002092 |
| 214988_s_at | SON | −1.18979 | 0.038913 |
| 207435_s_at | SRRM2 | −1.18845 | 0.036592 |
| 208632_at | RNF10 | −1.18799 | 0.035257 |
| 212066_s_at | USP34 | −1.17323 | 0.023279 |
| 210962_s_at | AKAP9 | −1.16272 | 0.049469 |
| 200886_s_at | PGAM1 | −1.15299 | 0.025269 |
| 208671_at | TDE2 | −1.13748 | 0.044086 |
| 221558_s_at | LEF1 | −1.13652 | 0.040434 |
| 201298_s_at | C2ORF6 | 1.10614 | 0.044086 |
| 201090_x_at | K-ALPHA-1 | 1.122132 | 0.013768 |
| 201463_s_at | TALDO1 | 1.153043 | 0.036592 |
| 200887_s_at | STAT1 | 1.158455 | 0.014068 |
| 200976_s_at | TAX1BP1 | 1.159119 | 0.001736 |
| 208992_s_at | STAT3 | 1.160979 | 0.035257 |
| 218472_s_at | PELO | 1.163412 | 0.036968 |
| 213571_s_at | EIF4EL3 | 1.179849 | 0.029489 |
| 217965_s_at | HCNGP | 1.185044 | 0.039073 |
| 201649_at | UBE2L6 | 1.18955 | 0.017752 |
| 208723_at | USP11 | 1.190718 | 0.025722 |
| 212318_at | TNPO3 | 1.195193 | 0.048612 |
| 58696_at | RRP41 | 1.202337 | 0.013671 |
| 204034_at | ETHE1 | 1.212179 | 0.013671 |
| 203923_s_at | CYBB | 1.213779 | 0.049402 |
| 208735_s_at | CTDSP2 | 1.214295 | 0.021969 |
| 214730_s_at | GLG1 | 1.21962 | 0.026021 |
| 201118_at | PGD | 1.219825 | 0.047145 |
| 212274_at | UNK_AV705559 | 1.2259 | 0.047362 |
| 209949_at | NCF2 | 1.228547 | 0.049921 |
| 202841_x_at | OGFR | 1.239383 | 0.022468 |
| 201061_s_at | STOM | 1.241937 | 0.047362 |
| 208699_x_at | TKT | 1.242781 | 0.029469 |
| 202531_at | IRF1 | 1.259354 | 0.005709 |
| 202245_at | LSS | 1.26358 | 0.030584 |
| 211661_x_at | PTAFR | 1.264165 | 0.036051 |
| 218154_at | FLJ12150 | 1.26707 | 0.05075 |
| 200923_at | LGALS3BP | 1.268399 | 0.027662 |
| 207091_at | P2RX7 | 1.272341 | 0.034056 |
| 208881_x_at | IDI1 | 1.287605 | 0.03075 |
| 222218_s_at | PILRA | 1.291622 | 0.030584 |
| 204858_s_at | ECGF1 | 1.291887 | 0.014236 |
| 210176_at | TLR1 | 1.30228 | 0.007618 |
| 214179_s_at | NFE2L1 | 1.302375 | 0.039085 |
| 202307_s_at | TAP1 | 1.312681 | 0.034618 |
| 209969_s_at | STAT1 | 1.314643 | 0.015163 |
| 221581_s_at | WBSCR5 | 1.342728 | 0.020776 |
| 202847_at | PCK2 | 1.344139 | 0.036592 |
| 210784_x_at | LILRB3 | 1.347846 | 0.028419 |
| 201945_at | FURIN | 1.347961 | 0.028718 |
| 211133_x_at | LILRB3 | 1.348999 | 0.00449 |
| 202510_s_at | TNFAIP2 | 1.354561 | 0.036968 |
| 209417_s_at | IFI35 | 1.367097 | 0.012523 |
| 219788_at | PILRA | 1.37054 | 0.046606 |
| 202068_s_at | LDLR | 1.387745 | 0.002092 |
| 211135_x_at | LILRB3 | 1.416291 | 0.011477 |
| 44673_at | SN | 1.425142 | 0.015037 |
| 202308_at | SREBF1 | 1.43555 | 0.040306 |
| 202193_at | LIMK2 | 1.456929 | 0.044938 |
| 216841_s_at | SOD2 | 1.462923 | 0.011477 |
| 215051_x_at | AIF1 | 1.464495 | 0.035257 |
| 204929_s_at | VAMP5 | 1.471584 | 0.026021 |
| 210146_x_at | LILRB2 | 1.47263 | 0.018928 |
| 202269_x_at | GBP1 | 1.474787 | 0.017817 |
| 204224_s_at | GCH1 | 1.480101 | 0.010146 |
| 210754_s_at | LYN | 1.482456 | 0.025074 |
| 207697_x_at | LILRB2 | 1.483562 | 0.010543 |
| 203922_s_at | CYBB | 1.520402 | 0.012857 |
| 205992_s_at | IL15 | 1.522262 | 0.005719 |
| 212907_at | SLC30A1 | 1.526797 | 0.029489 |
| 202626_s_at | LYN | 1.540308 | 0.004531 |
| 205322_s_at | MTF1 | 1.553477 | 0.00449 |
| 207277_at | CD209 | 1.574084 | 0.046606 |
| 215223_s_at | SOD2 | 1.583933 | 0.013369 |
| 208373_s_at | P2RY6 | 1.592741 | 0.00449 |
| 213716_s_at | SECTM1 | 1.60269 | 0.00449 |
| 205872_x_at | UNK_NM_022359 | 1.628734 | 0.005399 |
| 202917_s_at | S100A8 | 1.662116 | 0.028907 |
| 208962_s_at | UNK_BE540552 | 1.666732 | 0.010954 |
| 208963_x_at | FADS1 | 1.667884 | 0.034056 |
| 206025_s_at | TNFAIP6 | 1.671432 | 0.020946 |
| 219159_s_at | SLAMF7 | 1.735995 | 0.01107 |
| 216336_x_at | UNK_AL031602 | 1.748362 | 0.010543 |
| 206637_at | GPR105 | 1.796631 | 0.017817 |
| 208071_s_at | LAIR1 | 1.820282 | 0.014236 |
| 221165_s_at | IL22 | 1.835412 | 0.028907 |
| 206026_s_at | TNFAIP6 | 1.86622 | 0.039379 |
| 213629_x_at | MT1F | 1.953231 | 0.002803 |
| 210524_x_at | UNK_AF078844 | 1.984203 | 0.001736 |
| 204326_x_at | UNK_NM_002450 | 2.024194 | 0.00449 |
| 212859_x_at | MT2A | 2.113989 | 0.003571 |
| 210029_at | INDO | 2.207173 | 0.029489 |
| 204745_x_at | MT1G | 2.215332 | 0.00293 |
| 207533_at | CCL1 | 2.229332 | 0.036563 |
| 214038_at | UNK_AI984980 | 2.288964 | 0.027071 |
| 212185_x_at | MT2A | 2.359419 | 0.002803 |
| 202859_x_at | IL8 | 2.420166 | 0.010146 |
| 219519_s_at | SN | 2.441302 | 0.009444 |
| 211456_x_at | UNK_AF333388 | 2.494325 | 0.001736 |
| 217165_x_at | MT1F | 2.496014 | 0.00449 |
| 206461_x_at | MT1H | 2.575928 | 0.001736 |
| 208581_x_at | MT1X | 2.59979 | 0.002092 |
| 213515_x_at | HBG2 | 3.232958 | 0.036563 |
| 204419_x_at | HBG2 | 3.420226 | 0.039379 |

We claim:

1. A method for assessing an asthma-associated biological response in a sample from a patient, the method comprising the steps of:
(a) exposing a sample derived from a patient to an allergen in vitro;
(b) detecting a level of expression of at least one marker that is differentially expressed in asthma;
(c) comparing the level of expression of the at least one marker in the patient to a reference expression level of the at least one marker; and
(d) assessing an asthma-associated biological response based on the comparison done in step (c);
wherein the marker is not a cytokine gene or cytokine gene product.

2. The method of claim 1 wherein a difference between the level of expression of the at least one marker in the patient and the reference expression level of the at least one marker indicates the asthma-associated biological response.

3. The method of claim 1, wherein the reference expression level is the expression level in a sample from the patient not exposed to the allergen in vitro.

4. The method of claim 1 further comprising the step of contacting the sample with an agent before step (b);
wherein the assessment comprises evaluating the capability of the agent to modulate expression of the at least one marker.

5. The method of claim 1 further comprising the step of selecting a treatment for asthma following the assessment made in step (d).

6. The method of claim 5 wherein the treatment is selected from the group consisting of drug therapy, gene therapy, immunotherapy, radiation therapy, biological therapy, and surgery.

7. The method of claim 5 wherein the treatment is selected from the group consisting of an anti-histamine, a steroid, an immunomodulator, an IgE downregulator, an immunosuppressant, a bronchodilator/beta-2 agonist, an adenosine A2a receptor agonist, a leukotriene antagonist, a thromboxane A2 synthesis inhibitor, a 5-lipoxygenase inhibitor, an anti-cholinergic, a K+ channel opener, a VLA-4 antagonist, a neurokine antagonist, theophylline, a thromboxane A2 receptor antagonist, a beta-2 adrenoceptor agonist, a soluble interleukin receptor, a 5-lipoxygenase activating protein inhibitor, an arachidonic acid antagonist, an anti-inflammatory, a membrane channel inhibitor, an anti-interleukin antibody, a PDE-4 inhibitor, and a protease inhibitor.

8. The method of claim 5, wherein the selected treatment is a treatment that dampens the asthma-associated biological response.

9. The method of claim 1 wherein the at least one marker is selected from the group comprising the markers in Table 7b.

10. The method of claim 9 wherein the at least one marker is selected from the group comprising the markers in Table 7b with a false discovery rate (FDR) for association with asthma in peripheral blood mononuclear cells (PBMCs) prior to culture of less than 0.051.

11. The method of claim 1 further comprising the steps of:
(e) exposing the sample derived from the patient to an agent;
(f) detecting an expression level of the at least one marker in the sample exposed to the agent;
(g) comparing the expression level of the at least one marker in the sample exposed to the agent to either (i) the expression level of the at least one marker in the sample, or (ii) the reference expression level of the at least one marker; and
(h) assessing the modulation of the expression of the at least one marker by the agent;
wherein the agent modulates expression of the at least one marker when there is a difference between the expression level of the at least one marker in the sample exposed to the agent relative to either (i) the expression level of the at least one marker in the sample, (ii) the reference expression level of the at least one marker, or both (i) and (ii).

12. The method of claim 11 wherein at least one marker is selected from the group consisting of the markers set forth in Table 7b.

13. The method of claim 12 wherein the at least one marker is selected from a subset of the group consisting of the markers set forth in Table 7b having a false discovery rate (FDR) for association with asthma in PBMCs prior to culture of less than 0.051.

14. A method for diagnosis, prognosis or assessment of asthma in a patient, the method comprising the steps of assessing an asthma-associated biological response in a sample from the patient according to the method of claim 1; and providing a diagnosis, prognosis or assessment of asthma in the patient based on the assessment of the asthma-associated biological response in the sample.

15. The method of claim 14 wherein the wherein the diagnosis, prognosis or assessment of asthma in the patient is determined by the difference between the level of expression of the at least one marker in the patient and the reference expression level of the at least one marker.

16. The method of claim 14 wherein the reference expression level of the at least one marker is the expression level in a sample from the patient not exposed to the allergen in vitro.

17. A method for evaluating the effectiveness of an asthma treatment in a patient, the method comprising the steps of exposing the patient to the asthma treatment; and assessing an asthma-associated biological response in a sample from the patient according to the method of claim 1, wherein a dampened asthma-associated biological response is indicative of effectiveness of the asthma treatment.

18. The method of claim 17, wherein the asthma-associated biological response is compared to an asthma-associated biological response prior to treatment.

19. The method of claim 17, wherein the asthma-associated biological response is compared to a biological response in a sample from a healthy individual.

20. A method for evaluating the effectiveness of an asthma treatment in a patient, the method comprising the steps of:
(a) exposing a first sample from the patient to the asthma treatment;
(b) assessing a first asthma-associated biological response in the first sample from the patient; and
(c) assessing a second asthma-associated biological response in a second sample from the patient,
wherein the second sample is not exposed to the asthma treatment, and a dampened first asthma-associated biological response compared to the second asthma-associated response is indicative of the effectiveness of the asthma treatment.

21. The method of claim 20 wherein the first asthma-associated biological response is determined according to the method of claim 1.

22. The method of claim 20 wherein the second asthma-associated biological response is determined according to the method of claim 1.

23. A method for asthma diagnosis, prognosis or assessment, the method comprising comparing:
(a) a level of expression of at least one marker in a sample from a patient, wherein the at least one marker is selected from the group comprising the markers in Table 7b; and
(b) a reference level of expression of the marker;
wherein the comparison is indicative of the presence, absence, or status of asthma in a patient.

24. The method of claim 23 wherein a difference in the level of expression of the at least one marker in a sample from a patient relative to the reference level of expression of the at least one marker indicates a diagnosis, prognosis or assessment of asthma.

25. The method of claim 23 wherein the sample from the patient comprises peripheral blood mononuclear cells (PBMCs).

26. The method of claim 23 wherein the difference in the level of expression between the at least one marker from the patient sample and the reference level of the marker is at least 1.5 fold.

27. The method of claim 23 wherein the at least one marker is selected from the group comprising the markers in Table 7b having an FDR for association with asthma in PBMCs prior to culture of less than 0.051.

28. A method for evaluating the effectiveness of an asthma treatment in a patient, the method comprising:
(a) detecting an expression level of at least one marker in a sample derived from the patient during the course of treatment of the patient; and
(b) comparing the expression level in the patient to a reference expression level of the at least one marker;
wherein the difference between the detected expression level in the patient and the reference expression level is indicative of the effectiveness of the treatment of the patient's asthma; and
wherein the at least one marker is selected from the group comprising the markers in Table 7b.

29. The method of claim 28 wherein the sample derived from the patient comprises PBMCs.

30. The method of claim 28 wherein the at least one marker is selected from the group comprising the markers in Table 7b having an FDR for association with asthma in PBMCs prior to culture of less than 0.051.

31. The method of claim 28 wherein the reference expression level is the expression level of the at least one marker in a sample derived from the patient prior to the patient receiving the asthma treatment.

32. The method of claim 28, wherein the reference expression profile level of the at least one marker is the expression level in a sample from a healthy individual.

33. A method for selecting a treatment for asthma, comprising the steps of:
(a) detecting an expression level of at least one marker in a sample derived from a patient;
(b) comparing the expression level to a reference expression level of the marker;
(c) diagnosing the patient as having asthma; and
(d) selecting a treatment for the patient;
wherein the at least one marker is selected from the group comprising the markers in Table 7b.

34. The method of claim 33 wherein the reference expression profile level of the at least one marker is the expression level in a sample from a healthy individual.

35. The method of claim 33 wherein the sample derived from the patient comprises peripheral blood mononuclear cells (PBMCs).

36. The method of claim 33 wherein the treatment is selected from the group comprising drug therapy, gene therapy, immunotherapy, radiation therapy, biological therapy, and surgery.

37. The method of claim 33 wherein the treatment is selected from the group comprising an anti-histamine, a steroid, an immunomodulator, an IgE downregulator, an immunosuppressant, a bronchodilator/beta-2 agonist, an adenosine A2a receptor agonist, a leukotriene antagonist, a thromboxane A2 synthesis inhibitor, a 5-lipoxygenase inhibitor, an anti-cholinergic, a K+ channel opener, a VLA-4 antagonist, a neurokine antagonist, theophylline, a thromboxane A2 receptor antagonist, a beta-2 adrenoceptor agonist, a soluble interleukin receptor, a 5-lipoxygenase activating protein inhibitor, an arachidonic acid antagonist, an anti-inflammatory, a membrane channel inhibitor, an anti-interleukin antibody, a PDE-4 inhibitor, and a protease inhibitor.

38. The method according to claim 33 wherein the at least one marker is selected from the group consisting of the markers in Table 7b with an FDR for association with asthma in PBMCs prior to culture of less than 0.051.

39. A method for selecting a treatment for asthma, comprising the steps of:
(a) detecting an expression level of at least one marker in a sample derived from a patient;
(b) comparing the expression level of the at least one marker in the sample derived from a patient to a reference expression level of the at least one marker;
(c) determining whether the patient has asthma; and
(d) selecting a treatment for the patient having asthma;
wherein:
(i) a difference between the expression level of the at least one marker and the reference expression level of the at least one marker determines the patient having asthma; and
(ii) at least one marker is selected from the group consisting of the markers set forth in Table 7b.

40. The method of claim 39 wherein the reference expression profile level of the at least one marker is the expression level in a sample from a healthy individual.

41. The method of claim 39 wherein the sample derived from the patient comprises peripheral blood mononuclear cells (PBMCs).

42. The method of claim 39 wherein the treatment is selected from the group consisting of drug therapy, gene therapy, immunotherapy, radiation therapy, biological therapy, and surgery.

43. The method of claim 39 wherein the treatment is selected from the group consisting of an anti-histamine, a steroid, an immunomodulator, an IgE downregulator, an immunosuppressant, a bronchodilator/beta-2 agonist, an adenosine A2a receptor agonist, a leukotriene antagonist, a thromboxane A2 synthesis inhibitor, a 5-lipoxygenase inhibitor, an anti-cholinergic, a K⁺ channel opener, a VLA-4 antagonist, a neurokine antagonist, theophylline, a thromboxane A2 receptor antagonist, a beta-2 adrenoceptor agonist, a soluble interleukin receptor, a 5-lipoxygenase activating protein inhibitor, an arachidonic acid antagonist, an anti-inflammatory, a membrane channel inhibitor, an anti-interleukin antibody, a PDE-4 inhibitor, and a protease inhibitor.

44. A method for identifying or evaluating agents capable of modulating expression of at least one marker differentially expressed in asthma, comprising the steps of:
(a) exposing one or more cells to an agent;
(b) determining an expression level of the at least one marker in the exposed cells; and
(c) comparing the expression level of the marker with a reference expression level of the marker;
wherein said reference expression level is the expression level of the marker in a cell not exposed to the agent; and
wherein a change in the expression level of the at least one marker compared to the reference expression level is indicative that the agent is capable of modulating the expression level of the at least one marker; and
wherein the at least one marker is selected from the group comprising the markers in Table 7b.

45. The method of claim 44 wherein the cells contacted with the agent are PBMCs.

46. The method of claim 44 wherein the at least one marker is selected from the group comprising the markers in Table 7b having an FDR for association with asthma in PBMCs prior to culture of less than 0.051.

47. A method for identifying or evaluating agents capable of modulating an expression level of at least one marker differentially expressed in asthma, comprising the steps of:
(a) administering an agent to a human or a non-human mammal;
(b) determining the expression level of the at least one marker from the treated human or the treated non-human mammal;
(c) comparing the expression level of the marker with a reference expression level of the marker; and
(d) identifying or evaluating the agent as capable of modulating the expression level of the at least one marker in the human or animal based upon the comparison performed in step (c);
wherein the reference expression level is the expression level of the marker in an untreated human or untreated non-human animal; and
wherein the at least one marker is selected from the group comprising the markers in Table 7b.

48. The method of claim 47 wherein the agent is administered to a human.

49. The method of claim 47 wherein the at least one marker is selected from the group comprising the markers in Table 7b with an FDR for association with asthma in PBMCs prior to culture of less than 0.051.

50. An array for use in diagnosis, prognosis or assessment of asthma in a patient, comprising a plurality of addresses, each of which comprises a probe disposed thereon, wherein at least 15% of the plurality of addresses has disposed thereon probes that can specifically detect a marker of asthma in PBMCs or other tissues.

51. The array of claim 50 wherein the marker of asthma comprises at least one marker selected from the group consisting of the markers set forth in Tables 6, 7a, 7b, 8a, and 8b.

52. The array of claim 51 wherein the marker of asthma comprises at least one marker selected from the group consisting of the markers set forth in Table 7b having an FDR for association with asthma in PBMCs prior to culture.
